SEQUENCE LISTING (95 sequences)

SEQ ID NO: 1 - protein, 54 aa, Homo sapiens
MLQMAGQCSQNEYFDSLLHACIPCQLRCSSNTPPLTCQRYCNASVTNSVKGTNA

SEQ ID NO: 2 - protein, 54 aa, Artificial Sequence
  Mutant-1 human BCMA antigen
MLQMAGQCSQNEYFDSGGHACIPCQLRCSSNTPPLTCQRYCNASVTNSVKGTNA

SEQ ID NO: 3 - protein, 53 aa, Artificial Sequence
  Mutant-2 human BCMA antigen
MLQMAGQCSQNEYFDSLLHACIPCQLRCSSNPPGTCQRYCNASVTNSVKGTNA

SEQ ID NO: 4 - protein, 250 aa, Homo sapiens [sequence omitted]

SEQ ID NO: 5 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 6 - protein, 118 aa, Artificial Sequence
  Anti-BCMA-2C5 heavy chain variable region
EVQLVESGGGLVKPGGSLRLSCAASGFTSSTAWMSWVRQAPGKGLEWVGRIKSKSDGGTTDYAAPVKGRFTISRDDSKNTLFLQMNSLKTEDTAVYYCAKGGGTYGYWGQGTTVTVSS

SEQ ID NO: 7 - protein, 108 aa, Artificial Sequence
  Anti-BCMA-2C5 heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHT

SEQ ID NO: 8 - protein, 110 aa, Artificial Sequence
  Anti-BCMA-2C5 light chain variable region
QSALTQPASVSGSPGQSVTISCTGTSSAHGGHYYVSWYQQHPGKAPKLMIYDVSNRPSGVSNRFSGSKSGNTASLTISGLQAEDEADYYCGSYTSSGSYVFGTGTKLTVL

SEQ ID NO: 9 - protein, 110 aa, Artificial Sequence
  Anti-BCMA-BC7A light chain variable region
SYVLTQPASVSGSPGQSVTISCTGTSSDVGGYNYVSWYQQHPGKAPKLMIYDVSNRPSGVSNRFSGSKSGNTASLTISGLQAEDEADYYCGSYTSSGSYVFGTGTKLTVL

SEQ ID NO: 10 - protein, 216 aa, Artificial Sequence
  Anti-BCMA-BC7A (variant QSVLT - lambda) light chain variable region
QSVLTQPASVSGSPGQSVTISCTGTSSAHGGHYYVSWYQQHPGKAPKLMIYDVSNRPSGVSNRFSGSKSGNTASLTISGLQAEDEADYYCGSYTSSGSYVFGTGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 106 aa, Artificial Sequence
  Anti-BCMA-2C5 light chain constant region
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 12 - protein, 118 aa, Artificial Sequence; identical to SEQ ID NO: 6
  Anti-BCMA-2E1 heavy chain variable region

SEQ ID NO: 13 - protein, 110 aa, Artificial Sequence
  Anti-BCMA-2E1 light chain variable region
QSALTQPASVSGSPGQSVTISCTGTSSDGGGHTYVSWYQQHPGKAPKLMIYDVSNRPSWVSNRFSGSKSGNTASLTISGLQAEDEADYYCGSYTSSGSYVFGTGTKLTVL

SEQ ID NO: 14 - protein, 113 aa, Artificial Sequence
  Anti-BCMA-BC4C9 heavy chain variable region
EVQLVQSGAEVKKPGASVKVSCKAFGYTFTSYDINWVRQATGQGLEWMGWMNPNSGNTGYAQKFQGRVTMTRNTSISTAYMELSSLRSEDTAVYYCASGLGEWGQGTLVTVSS

SEQ ID NO: 15 - protein, 111 aa, Artificial Sequence
  Anti-BCMA-BC4C9 light chain variable region
QSVLTQPASVSGSPGQSVTISCTGTSSDVGGYNYVSWYQQHPGKAPKLMIYDVSKRPSGVSNRFSGSKSGNTASLTISGLQAEDEADYYCISYSSSSTFYVFGTGTKVTVL

SEQ ID NO: 16 - protein, 117 aa, Artificial Sequence
  Anti-BCMA-BC5C4 heavy chain variable region
EVQLVESGGGLVQPGRSLRLSCAASGFTFDDYAMHWVRQAPGKGLEWVSGISWNSGSIGYADSVKGRFTISRDNAKNSLYLQMNSLRAEDTAVYYCATIDNVAFHSWGQGTLVTVSS

SEQ ID NO: 17 - protein, 111 aa, Artificial Sequence
  Anti-BCMA-BC5C4 light chain variable region
QSVLTQPPSASGSPGQSVTISCTGTSSDIGGYNYVSWYQQHPGKAPKLIIYEVSNRPSGVSDRFSGSKSGNTASLTISGLQAEDEADYYCSSYTDNGALVVFGGGTKLTVL

SEQ ID NO: 18 - protein, 120 aa, Artificial Sequence
  Anti-BCMA-BC6G8 heavy chain variable region
QVQLQQSGPGLVKPSQTLSLTCAISGDSVSSNSVGWHWIRQSPSRGLEWLGRTYYRSNFATDYAASVRGRMTINADTSTNQISLHLNSLTPEDTAVYYCTRDWYGVYDFWGQGTLVTVSS

SEQ ID NO: 19 - protein, 109 aa, Artificial Sequence
  Anti-BCMA-BC6G8 light chain variable region
SYELMQPPSVSVAPGKTARITCGGNNIGSKSVHWYQQKPGQAPVLVIYYDSDRPSGIPERFSGSNSGNTATLTISRVEAGDEADYYCQVWDSSSDHLVVFGGGTKLTVL

SEQ ID NO: 20 - protein, 118 aa, Artificial Sequence; identical to SEQ ID NO: 6
  Anti-BCMA-2D11 heavy chain variable region

SEQ ID NO: 21 - protein, 110 aa, Artificial Sequence
  Anti-BCMA-2D11 light chain variable region
SYELTQPASVSGSPGQSVTISCTGTSSVVGGHDYVSWYQQHPGKAPKLMIYDVSNRPSGVSNRFSGSKSGNTASLTISGLQAEDEADYYCGSYTSSGSYVFGTGTKVTVL

SEQ ID NO: 22 - protein, 118 aa, Artificial Sequence; identical to SEQ ID NO: 6
  Anti-BCMA-2G2 heavy chain variable region

SEQ ID NO: 23 - protein, 110 aa, Artificial Sequence
  Anti-BCMA-2G2 light chain variable region
QSVLTQPASVSGSPGQSVTISCTGTSSSVGGRQYVSWYQQHPGKAPKLMIYDVSNRPSGVSNRFSGSKSGNTASLTISGLQAEDEADYYCGSYTSSGSYVFGTGTKLTVL

SEQ ID NO: 24 - protein, 118 aa, Artificial Sequence; identical to SEQ ID NO: 6
  Anti-BCMA-2D8 heavy chain variable region

SEQ ID NO: 25 - protein, 110 aa, Artificial Sequence
  Anti-BCMA-2D8 light chain variable region
QSVLTQPASVSGSPGQSVTISCTGTSSSIGDSYYVSWYQQHPGKAPKLMIYDVSNRPSGVSNRFSGSKSGNTASLTISGLQAEDEADYYCGSYTSSGSYVFGTGTTLTVL

SEQ ID NO: 26 - protein, 118 aa, Artificial Sequence; identical to SEQ ID NO: 6
  Anti-BCMA-2E8 heavy chain variable region

SEQ ID NO: 27 - protein, 110 aa, Artificial Sequence
  Anti-BCMA-2E8 light chain variable region
QSVLTQPASVSGSPGQSVTISCTGTSSDVGGYNYVSWYQQHPGKAPKLMIYDVSNRPSGVSNRFSGSKSGNTASLTISGLQAEDEADYYCGSLRSNGDYVFGTGTTLTVL

SEQ ID NO: 28 - protein, 117 aa, Artificial Sequence
  Anti-BCMA Bluebird (bb2121) heavy chain variable region
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTTLTVSS

SEQ ID NO: 29 - protein, 108 aa, Artificial Sequence; identical to SEQ ID NO: 7
  Anti-BCMA Bluebird (bb2121) heavy chain constant region

SEQ ID NO: 30 - protein, 111 aa, Artificial Sequence
  Anti-BCMA Bluebird (bb2121) light chain variable region
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 31 - protein, 107 aa, Artificial Sequence [sequence omitted]

SEQ ID NO: 32 - protein, 15 aa, Artificial Sequence
  CAR GS linker
GGGGSGGGGSGGGGS

SEQ ID NO: 33 - protein, 15 aa, Artificial Sequence
  CAR bb2121 linker
GSTSGSGKPGSGEGS

SEQ ID NO: 34 - protein, 48 aa, Artificial Sequence
  CD8 hinge
AKPTTTPAPRPPTPAPTIASQPLSLRPEACRPAAGGAVHTRGLDFAPR

SEQ ID NO: 35 - protein, 40 aa, Artificial Sequence
  CD28 hinge
KIEVMYPPPYLDNEKSNGTIIHVKGKHLCPSPLFPGPSKP

SEQ ID NO: 36 - protein, 88 aa, Artificial Sequence
  CD8 hinge + CD28 hinge (long hinge)
AKPTTTPAPRPPTPAPTIASQPLSLRPEACRPAAGGAVHTRGLDFAPRKIEVMYPPPYLDNEKSNGTIIHVKGKHLCPSPLFPGPSKP

SEQ ID NO: 37 - protein, 27 aa, Artificial Sequence
  CD28 transmembrane
FWVLVVVGGVLACYSLLVTVAFIIFWV

SEQ ID NO: 38 - protein, 23 aa, Artificial Sequence
  CD8 transmembrane
IYIWAPLAGTCGVLLLSLVITLY

SEQ ID NO: 39 - protein, 27 aa, Artificial Sequence
  4-1BB transmembrane
IISFFLALTSTALLFLLFFLTLRFSVV

SEQ ID NO: 40 - protein, 21 aa, Artificial Sequence
  CD3zeta transmembrane
LCYLLDGILFIYGVILTALFL

SEQ ID NO: 41 - protein, 42 aa, Artificial Sequence
  4-1BB co-stimulatory sequence
KRGRKKLLYIFKQPFMRPVQTTQEEDGCSCRFPEEEEGGCEL

SEQ ID NO: 42 - protein, 41 aa, Artificial Sequence
  CD28 co-stimulatory sequence
RSKRSRLLHSDYMNMTPRRPGPTRKHYQPYAPPRDFAAYRS

SEQ ID NO: 43 - protein, 42 aa, Artificial Sequence
  OX40 co-stimulatory sequence
ALYLLRRDQRLPPDAHKPPGGGSFRTPIQEEQADAHSTLAKI

SEQ ID NO: 44 - protein, 112 aa, Artificial Sequence
  CD3zeta ITAM 1, 2, 3
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 38 aa, Artificial Sequence
  CD3zeta ITAM 1
RVKFSRSADAPAYQQGQNQLYNELNLGRREEYDVLDKR

SEQ ID NO: 46 - protein, 47 aa, Artificial Sequence
  CD3zeta ITAM 2
RVKFSRSADRGRDPEMGGKPRRKNPQEGLYNELQKDKMAEAYSEIGM

SEQ ID NO: 47 - protein, 45 aa, Artificial Sequence
  CD3zeta ITAM 3
RVKFSRSADKGERRRGKGHDGLYQGLSTATKDTYDALHMQALPPR

SEQ ID NO: 48 - protein, 153 aa, Artificial Sequence
  DAR V1 intracellular domain
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 154 aa, Artificial Sequence
  DAR V2a intracellular domain
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 153 aa, Artificial Sequence; identical to SEQ ID NO: 48
  DAR V2b and CAR (28Z) intracellular domain

SEQ ID NO: 51 - protein, 195 aa, Artificial Sequence
  DAR V2c intracellular domain
KRGRKKLLYIFKQPFMRPVQTTQEEDGCSCRFPEEEEGGCELRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 87 aa, Artificial Sequence
  DAR V3a and V4 intracellular domain
KRGRKKLLYIFKQPFMRPVQTTQEEDGCSCRFPEEEEGGCELRVKFSRSADKGERRRGKGHDGLYQGLSTATKDTYDALHMQALPPR

SEQ ID NO: 53 - protein, 128 aa, Artificial Sequence
  DAR V3b intracellular domain
KRGRKKLLYIFKQPFMRPVQTTQEEDGCSCRFPEEEEGGCELRSKRSRLLHSDYMNMTPRRPGPTRKHYQPYAPPRDFAAYRSRVKFSRSADKGERRRGKGHDGLYQGLSTATKDTYDALHMQALPPR

SEQ ID NO: 54 - protein, 19 aa, Artificial Sequence
  Heavy chain leader sequence
MEWSWVFLFFLSVTTGVHS

SEQ ID NO: 55 - protein, 20 aa, Artificial Sequence
  Light chain leader sequence
MSVPTQVLGLLLLWLTDARC

SEQ ID NO: 56 - protein, 21 aa, Artificial Sequence
  Alternative leader sequence
MALPVTALLLPLALLLHAARP

SEQ ID NO: 57 - protein, 21 aa, Artificial Sequence
  T2A self-cleaving sequence
GSGEGRGSLLTCGDVEENPGP

SEQ ID NO: 58 - protein, 22 aa, Artificial Sequence
  P2A self-cleaving sequence
GSGATNFSLLKQAGDVEENPGP

SEQ ID NO: 59 - protein, 23 aa, Artificial Sequence
  E2A self-cleaving sequence
GSGQCTNYALLKLAGDVESNPGP

SEQ ID NO: 60 - protein, 25 aa, Artificial Sequence
  F2A self-cleaving sequence
GSGVKQTLNFDLLKLAGDVESNPGP

SEQ ID NO: 61 - protein, 530 aa, Artificial Sequence
  CAR 28Z BCMA-2C5
MEWSWVFLFFLSVTTGVHSQVQLVESGGGLVKPGGSLRLSCAASGFTSSTAWMSWVRQAPGKGLEWVGRIKSKSDGGTTDYAAPVKGRFTISRDDSKNTLFLQMNSLKTEDTAVYYCAKGGGTYGYWGQGTLVTVSSGGGGSGGGGSGGGGSSYVLTQPASVSGSPGQSVTISCTGTSSAHGGHYYVSWYQQHPGKAPKLMIYDVSNRPSGVSNRFSGSKSGNTASLTISGLQAEDEADYYCGSYTSSGSYVFGTGTKLTVLAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 530 aa, Artificial Sequence
  CAR 28Z BCMA-bb2121
MEWSWVFLFFLSVTTGVHSDIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTTLTVSSAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 770 aa, Artificial Sequence
  DAR V1 BCMA-2C5 precursor
MEWSWVFLFFLSVTTGVHSEVQLVESGGGLVKPGGSLRLSCAASGFTSSTAWMSWVRQAPGKGLEWVGRIKSKSDGGTTDYAAPVKGRFTISRDDSKNTLFLQMNSLKTEDTAVYYCAKGGGTYGYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMSVPTQVLGLLLLWLTDARCSYVLTQPASVSGSPGQSVTISCTGTSSAHGGHYYVSWYQQHPGKAPKLMIYDVSNRPSGVSNRFSGSKSGNTASLTISGLQAEDEADYYCGSYTSSGSYVFGTGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 64 - protein, 494 aa, Artificial Sequence
  DAR V1 BCMA-2C5 1st polypeptide
EVQLVESGGGLVKPGGSLRLSCAASGFTSSTAWMSWVRQAPGKGLEWVGRIKSKSDGGTTDYAAPVKGRFTISRDDSKNTLFLQMNSLKTEDTAVYYCAKGGGTYGYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 216 aa, Artificial Sequence
  DAR V1 BCMA-2C5 2nd polypeptide
SYVLTQPASVSGSPGQSVTISCTGTSSAHGGHYYVSWYQQHPGKAPKLMIYDVSNRPSGVSNRFSGSKSGNTASLTISGLQAEDEADYYCGSYTSSGSYVFGTGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 66 - protein, 723 aa, Artificial Sequence
  DAR V2a BCMA-2C5 precursor
MEWSWVFLFFLSVTTGVHSEVQLVESGGGLVKPGGSLRLSCAASGFTSSTAWMSWVRQAPGKGLEWVGRIKSKSDGGTTDYAAPVKGRFTISRDDSKNTLFLQMNSLKTEDTAVYYCAKGGGTYGYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMSVPTQVLGLLLLWLTDARCSYVLTQPASVSGSPGQSVTISCTGTSSAHGGHYYVSWYQQHPGKAPKLMIYDVSNRPSGVSNRFSGSKSGNTASLTISGLQAEDEADYYCGSYTSSGSYVFGTGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 67 - protein, 447 aa, Artificial Sequence
  DAR V2a BCMA-2C5 1st polypeptide
EVQLVESGGGLVKPGGSLRLSCAASGFTSSTAWMSWVRQAPGKGLEWVGRIKSKSDGGTTDYAAPVKGRFTISRDDSKNTLFLQMNSLKTEDTAVYYCAKGGGTYGYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 216 aa, Artificial Sequence; identical to SEQ ID NO: 65
  DAR V2a BCMA-2C5 2nd polypeptide

SEQ ID NO: 69 - protein, 722 aa, Artificial Sequence
  DAR V2b BCMA-2C5 precursor
MEWSWVFLFFLSVTTGVHSEVQLVESGGGLVKPGGSLRLSCAASGFTSSTAWMSWVRQAPGKGLEWVGRIKSKSDGGTTDYAAPVKGRFTISRDDSKNTLFLQMNSLKTEDTAVYYCAKGGGTYGYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMSVPTQVLGLLLLWLTDARCSYVLTQPASVSGSPGQSVTISCTGTSSAHGGHYYVSWYQQHPGKAPKLMIYDVSNRPSGVSNRFSGSKSGNTASLTISGLQAEDEADYYCGSYTSSGSYVFGTGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 70 - protein, 446 aa, Artificial Sequence
  DAR V2b BCMA-2C5 1st polypeptide
EVQLVESGGGLVKPGGSLRLSCAASGFTSSTAWMSWVRQAPGKGLEWVGRIKSKSDGGTTDYAAPVKGRFTISRDDSKNTLFLQMNSLKTEDTAVYYCAKGGGTYGYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 216 aa, Artificial Sequence; identical to SEQ ID NO: 65
  DAR V2b BCMA-2C5 2nd polypeptide

SEQ ID NO: 72 - protein, 764 aa, Artificial Sequence
  DAR V2c BCMA-2C5 precursor
MEWSWVFLFFLSVTTGVHSEVQLVESGGGLVKPGGSLRLSCAASGFTSSTAWMSWVRQAPGKGLEWVGRIKSKSDGGTTDYAAPVKGRFTISRDDSKNTLFLQMNSLKTEDTAVYYCAKGGGTYGYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMSVPTQVLGLLLLWLTDARCSYVLTQPASVSGSPGQSVTISCTGTSSAHGGHYYVSWYQQHPGKAPKLMIYDVSNRPSGVSNRFSGSKSGNTASLTISGLQAEDEADYYCGSYTSSGSYVFGTGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 73 - protein, 488 aa, Artificial Sequence
  DAR V2c BCMA-2C5 1st polypeptide
EVQLVESGGGLVKPGGSLRLSCAASGFTSSTAWMSWVRQAPGKGLEWVGRIKSKSDGGTTDYAAPVKGRFTISRDDSKNTLFLQMNSLKTEDTAVYYCAKGGGTYGYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 216 aa, Artificial Sequence; identical to SEQ ID NO: 65
  DAR V2c BCMA-2C5 2nd polypeptide

SEQ ID NO: 75 - protein, 656 aa, Artificial Sequence
  DAR V3a BCMA-2C5 precursor
MEWSWVFLFFLSVTTGVHSEVQLVESGGGLVKPGGSLRLSCAASGFTSSTAWMSWVRQAPGKGLEWVGRIKSKSDGGTTDYAAPVKGRFTISRDDSKNTLFLQMNSLKTEDTAVYYCAKGGGTYGYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADKGERRRGKGHDGLYQGLSTATKDTYDALHMQALPPRGSGEGRGSLLTCGDVEENPGPMSVPTQVLGLLLLWLTDARCSYVLTQPASVSGSPGQSVTISCTGTSSAHGGHYYVSWYQQHPGKAPKLMIYDVSNRPSGVSNRFSGSKSGNTASLTISGLQAEDEADYYCGSYTSSGSYVFGTGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 76 - protein, 380 aa, Artificial Sequence
  DAR V3a BCMA-2C5 1st polypeptide
EVQLVESGGGLVKPGGSLRLSCAASGFTSSTAWMSWVRQAPGKGLEWVGRIKSKSDGGTTDYAAPVKGRFTISRDDSKNTLFLQMNSLKTEDTAVYYCAKGGGTYGYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADKGERRRGKGHDGLYQGLSTATKDTYDALHMQALPPR

SEQ ID NO: 77 - protein, 216 aa, Artificial Sequence; identical to SEQ ID NO: 65
  DAR V3a BCMA-2C5 2nd polypeptide

SEQ ID NO: 78 - protein, 697 aa, Artificial Sequence
  DAR V3b BCMA-2C5 precursor
MEWSWVFLFFLSVTTGVHSEVQLVESGGGLVKPGGSLRLSCAASGFTSSTAWMSWVRQAPGKGLEWVGRIKSKSDGGTTDYAAPVKGRFTISRDDSKNTLFLQMNSLKTEDTAVYYCAKGGGTYGYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADKGERRRGKGHDGLYQGLSTATKDTYDALHMQALPPRGSGEGRGSLLTCGDVEENPGPMSVPTQVLGLLLLWLTDARCSYVLTQPASVSGSPGQSVTISCTGTSSAHGGHYYVSWYQQHPGKAPKLMIYDVSNRPSGVSNRFSGSKSGNTASLTISGLQAEDEADYYCGSYTSSGSYVFGTGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 79 - protein, 421 aa, Artificial Sequence
  DAR V3b BCMA-2C5 1st polypeptide
EVQLVESGGGLVKPGGSLRLSCAASGFTSSTAWMSWVRQAPGKGLEWVGRIKSKSDGGTTDYAAPVKGRFTISRDDSKNTLFLQMNSLKTEDTAVYYCAKGGGTYGYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADKGERRRGKGHDGLYQGLSTATKDTYDALHMQALPPR

SEQ ID NO: 80 - protein, 216 aa, Artificial Sequence; identical to SEQ ID NO: 65
  DAR V3b BCMA-2C5 2nd polypeptide

SEQ ID NO: 81 - protein, 616 aa, Artificial Sequence
  DAR V4 BCMA-2C5 precursor
MEWSWVFLFFLSVTTGVHSEVQLVESGGGLVKPGGSLRLSCAASGFTSSTAWMSWVRQAPGKGLEWVGRIKSKSDGGTTDYAAPVKGRFTISRDDSKNTLFLQMNSLKTEDTAVYYCAKGGGTYGYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTFWVLVVVGGVLACYSLLVTVAFIIFWVKRGRKKLLYIFKQPFMRPVQTTQEEDGCSCRFPEEEEGGCELRVKFSRSADKGERRRGKGHDGLYQGLSTATKDTYDALHMQALPPRGSGEGRGSLLTCGDVEENPGPMSVPTQVLGLLLLWLTDARCSYVLTQPASVSGSPGQSVTISCTGTSSAHGGHYYVSWYQQHPGKAPKLMIYDVSNRPSGVSNRFSGSKSGNTASLTISGLQAEDEADYYCGSYTSSGSYVFGTGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 82 - protein, 340 aa, Artificial Sequence
  DAR V4 BCMA-2C5 1st polypeptide
EVQLVESGGGLVKPGGSLRLSCAASGFTSSTAWMSWVRQAPGKGLEWVGRIKSKSDGGTTDYAAPVKGRFTISRDDSKNTLFLQMNSLKTEDTAVYYCAKGGGTYGYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTFWVLVVVGGVLACYSLLVTVAFIIFWVKRGRKKLLYIFKQPFMRPVQTTQEEDGCSCRFPEEEEGGCELRVKFSRSADKGERRRGKGHDGLYQGLSTATKDTYDALHMQALPPR

SEQ ID NO: 83 - protein, 216 aa, Artificial Sequence; identical to SEQ ID NO: 65
  DAR V4 BCMA-2C5 2nd polypeptide

SEQ ID NO: 84 - protein, 724 aa, Artificial Sequence
  DAR V2a BCMA-bb2121 precursor
MEWSWVFLFFLSVTTGVHSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMSVPTQVLGLLLLWLTDARCDIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 446 aa, Artificial Sequence
  DAR V2a BCMA-bb2121 1st polypeptide
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 218 aa, Artificial Sequence
  DAR V2a BCMA-bb2121 2nd polypeptide
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 86 aa, Artificial Sequence
  DAR V3c intracellular domain
RSKRSRLLHSDYMNMTPRRPGPTRKHYQPYAPPRDFAAYRSRVKFSRSADKGERRRGKGHDGLYQGLSTATKDTYDALHMQALPPR

SEQ ID NO: 88 - protein, 195 aa, Artificial Sequence
  DAR V2c-alt intracellular domain
RSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 128 aa, Artificial Sequence
  DAR V3b-alt intracellular domain
RSKRSRLLHSDYMNMTPRRPGPTRKHYQPYAPPRDFAAYRSKRGRKKLLYIFKQPFMRPVQTTQEEDGCSCRFPEEEEGGCELRVKFSRSADKGERRRGKGHDGLYQGLSTATKDTYDALHMQALPPR

SEQ ID NO: 90 - protein, 19 aa, Mus musculus
MEWSWVFLFFLSVTTGVHS

SEQ ID NO: 91 - protein, 10 aa, Artificial Sequence
  IgG1 upper hinge sequence
EPKSCDKTHT

SEQ ID NO: 92 - protein, 4 aa, Artificial Sequence
  IgG1 core hinge sequence
  MISC_FEATURE (3)..(3): X = P, R or S
CPXC

SEQ ID NO: 93 - protein, 9 aa, Artificial Sequence
  Lower hinge/CH2 sequence
PAPELLGGP

SEQ ID NO: 94 - protein, 12 aa, Artificial Sequence
  Exemplary Fc region (CH2)
SVFLFPPKPKDT

SEQ ID NO: 95 - protein, 23 aa, Artificial Sequence
  Hinge region including an upper, core and lower hinge
EPKSCDKTHTCPPCPAPELLGGP

(19) United States
(12) Patent Application Publication
(10) Pub. No.: US 2022/0251168 A1
(43) Pub. Date: Aug. 11, 2022

(54) Dimeric Antigen Receptors (DAR) that Bind BCMA

(71) Applicant: Sorrento Therapeutics, Inc., San Diego, CA (US)

(72) Inventors: Henry Hongjun Ji, Rancho Santa Fe, CA (US); Wenzhong Guo, San Diego, CA (US); Yanliang Zhang, San Diego, CA (US); Bei Bei Ding, San Diego, CA (US); Gunnar F. Kaufmann, San Diego, CA (US)

(73) Assignee: Sorrento Therapeutics, Inc., San Diego, CA (US)

(21) Appl. No.: 17/686,261

(22) Filed: Mar. 3, 2022

Related U.S. Application Data
(63) Continuation of application No. PCT/US2020/049538, filed on Sep. 4, 2020.
(60) Provisional application No. 62/896,190, filed on Sep. 5, 2019.
(60) Provisional application No. 62/896,990, filed on Sep. 6, 2019.
(60) Provisional application No. 62/910,341, filed on Oct. 3, 2019.
(60) Provisional application No. 62/943,069, filed on Dec. 3, 2019.
(60) Provisional application No. 63/030,145, filed on May 26, 2020.

(51) Int. Cl. C07K 14/725; A61K 35/17

(52) U.S. Cl. CPC C07K 14/7051; A61K 38/00; A61K 35/17

(57) ABSTRACT

The present disclosure provides dimeric antigen receptors (DAR) constructs that bind a BCMA target antigen, where the DAR construct comprises a heavy chain binding region on one polypeptide chain and a light chain binding region on a separate polypeptide chain. The two polypeptide chains that make up the dimeric antigen receptors can dimerize to form an antigen binding domain. The dimeric antigen receptors have antibody-like properties as they bind specifically to a target antigen. The dimeric antigen receptors can be used for directed cell therapy.

### RELATED APPLICATIONS

This application is a continuation of International Patent Application No. PCT/US2020/049538, filed Sep. 4, 2020, which claims the benefit of priority to U.S. Provisional Patent Application No. 62/896,190, filed on Sep. 5, 2019, to U.S. Provisional Patent Application No. 62/896,990, filed Sep. 6, 2019, to U.S. Provisional Patent Application No. 62/910,341, filed Oct. 3, 2019, to U.S. Provisional Patent Application No. 62/943,069, filed Dec. 3, 2019, and to U.S. Provisional Patent Application No. 63/030,145, filed May 26, 2020, the entire contents of each of which are expressly incorporated herein by reference.

### SEQUENCE LISTING

The instant application contains a Sequence Listing which has been submitted electronically in ASCII format and is hereby incorporated by reference in its entirety. Said ASCII copy, created on Jul. 24, 2020, is named 2020-07-24_01223-0012-00PCT_Sequence_Listing_ST25.txt and is 167,936 bytes in size.

### TECHNICAL FIELD

The present disclosure provides dimeric antigen receptors (DAR) protein constructs that bind specifically to a target antigen, nucleic acids that encode the dimeric antigen receptors, vectors comprising the nucleic acids, and host cells harboring the vectors.

### BACKGROUND AND SUMMARY

Chimeric antigen receptors (CARs) have been developed to target antigens associated, in particular, with cancer. The first-generation CAR was engineered to contain a signaling domain (TCRζ) that delivers an activation stimulus (signal 1) only (Geiger et al., J. Immunol. 162(10): 5931-5939, 1999; Haynes et al., J. Immunol. 166(1): 182-187, 2001) (Hombach et al. Cancer Res. 61(5): 1976-1982, 2001; Hombach et al., J. Immunol. 167(11): 6123-6131, 2001; Maher et al., Nat. Biotechnol. 20(1): 70-75, 2002). T cells grafted with the first-generation CARs alone exhibited limited anti-tumor efficacy due to suboptimal activation (Beecham et al., J. Immunother. 23(6): 631-642, 2000). The second-generation CAR, immunoglobulin-CD28-T cell receptor (IgCD28TCR), incorporated a costimulatory CD28 (signal 2) into the first-generation receptor (Gerstmayer et al., J. Immunol. 158(10): 4584-4590, 1997; Emtage et al., Clin. Cancer Res. 14(24): 8112-8122, 2008; Lo, Ma et al., Clin. Cancer Res. 16(10): 2769-2780, 2010) that resulted in CAR-T cells with a greater anti-tumor capacity (Finney et al., J. Immunol. 161(6): 2791-2797, 1998; Hombach et al., Cancer Res. 61(5): 1976-1982, 2001, Maher et al., Nat. Biotechnol. 20(1): 70-75, 2002). Various CAR variants have been developed by replacing the signal domains of TCRζ or CD28 with molecules with similar functions, such as FcRγ, 4-1BB and OX40 (Eshhar et al., Proc. Natl. Acad. Sci. USA 90(2): 720-724, 1993). TCR CAR-T cells against various tumor antigens have been developed (Ma et al., Cancer Gene Ther. 11(4): 297-306, 2004; Ma et al., Prostate 61(1): 12-25, 2004; Lo et al., Clin. Cancer Res. 16(10): 2769-2780, 2010; Kong et al., Clin. Cancer Res. 18(21): 5949-5960, 2012; Ma et al., Prostate 74(3): 286-296, 2014; Katz et al., Clin. Cancer Res. 21(14): 3149-3159, 2015; Junghans et al., 2016 The Prostate, 76(14):1257-1270).

Adoptive immunotherapy by infusion of T cells engineered with chimeric antigen receptors (CARs) for redirected tumoricidal activity represents a potentially highly specific modality for the treatment of metastatic cancer. CAR-T cells targeting CD19, a molecule expressed on B cells, have shown success in treatment of B cell malignancies and have received FDA approval, with some trials showing a response rate of up to 70%, including sustained complete responses. Nonetheless, CAR-T cells may show nonspecific activation, which may result in potentially serious adverse events through inappropriate immune activity.

Thus, there remains a need in the art to harness the powerful efficacy of CAR treatments with increased specificity.

Antigen receptors comprising both an antibody heavy chain binding region and an antibody light chain binding region in separate polypeptide chains and their use in directed cell therapy are disclosed herein in an effort to meet this need and/or provide other benefits, or at least provide the public with a useful choice. In some embodiments, the present disclosure provides dimeric antigen receptors (DAR) comprising first and second polypeptide chains, e.g., that form a Fab fragment joined to transmembrane and intracellular regions, and cells expressing such DARs. In some embodiments, T cells expressing DARs can show target-specific expansion and cytotoxicity, e.g., in comparison to T cells expressing a traditional CAR. Embodiments according to this disclosure are set forth in the claims and the detailed description.

### DESCRIPTION OF THE DRAWINGS

FIG. 1A is a schematic showing an exemplary dimeric antigen receptor comprising two intracellular signaling sequences.

FIG. 1B is a schematic showing an exemplary dimeric antigen receptor comprising three intracellular signaling sequences.

FIG. 2A is a schematic showing an exemplary dimeric antigen receptor comprising two intracellular signaling sequences.

FIG. 2B is a schematic showing an exemplary dimeric antigen receptor comprising three intracellular signaling sequences.

FIG. 3A is a schematic showing an exemplary precursor polypeptide molecule comprising a self-cleaving sequence and three intracellular signaling sequences.

FIG. 3B is a schematic showing an exemplary precursor polypeptide molecule comprising a self-cleaving sequence and two intracellular signaling sequences.

FIG. 4A is a schematic showing an exemplary precursor polypeptide molecule comprising a self-cleaving sequence and three intracellular signaling sequences.

FIG. 4B is a schematic showing an exemplary precursor polypeptide molecule comprising a self-cleaving sequence and two intracellular signaling sequences.

FIG. 5A shows the results of a flow cytometry study comparing transgenic T cells (Donor 1) expressing two different versions of BCMA chimeric antigen receptor (CAR) constructs. The data was collected 13 days post-transfection. The negative control is a non-transgenic activated T cell (ATC). Another negative control is a TRAC-minus T cell line (T-cell receptor alpha constant-minus). The transfection efficiency and expression level flow cytometry study is described in Example 5.

FIG. 5B shows the results of a flow cytometry study (at day 11) comparing transgenic T cells (Donor 1) expressing three different versions of BCMA-2C5 dimeric antigen receptor (DAR) constructs. The negative control is a TRAC-minus T cell line from FIG. 5A. A comparison of transgenic cells expressing various DAR constructs is shown: DAR V2c construct; DAR V3a construct; and DAR V3b construct. The data was collected 13 days post-transfection. The transfection efficiency and expression level flow cytometry study is described in Example 5.

FIG. 6 is a graph showing the percent cytotoxicity of T cells (Donor 1) expressing BCMA CAR, or BCMA DAR, on RPMI 8226 target cells. Line A designates the negative control TRAC-minus T cell line (T-cell receptor alpha constant-minus); Line B designates the DAR BCMA-2C5 V2c construct; Line C designates the CAR bb2121 construct; Line D (dotted line) designates the DAR BCMA-2C5 V3a construct; Line E designates the DAR BCMA-2C5 V3b construct; and Line F designates the CAR BCMA-2C5 construct. The cytotoxicity study is described in Example 6.

FIG. 7A is a bar graph showing the level of IFN-gamma release (40 hours post-target stimulation) from a negative control TRAC-minus T cell line (T-cell receptor alpha constant-minus), or T cells (Donor 1) expressing: the CAR bb2121 construct; CAR BCMA-2C5 construct; DAR BCMA-2C5 V2c construct; DAR BCMA-2C5 V3a construct; or DAR BCMA-2C5 V3b construct. Each data set shows from left to right U266 cells (BCMA-positive cells), K562 cells (BCMA-negative cells), medium only, or RPMI 8226 cells (BCMA-positive cells). The cytokine release study is described in Example 7.

FIG. 7B is a bar graph showing the level of GM-CSF release (40 hours post-target stimulation) from a negative control TRAC-minus T cell line (T-cell receptor alpha constant-minus), or T cells (Donor 1) expressing: the CAR bb2121 construct; CAR BCMA-2C5 construct; DAR BCMA-2C5 V2c construct; DAR BCMA-2C5 V3a construct; or DAR BCMA-2C5 V3b construct. Each data set shows from left to right U266 cells (BCMA-positive cells), K562 cells (BCMA-negative cells), medium only, or RPMI 8226 cells (BCMA-positive cells). The cytokine release study is described in Example 7.

FIG. 8A shows the results of a flow cytometry study comparing expansion capability of negative control TRAC-minus T cell line (T-cell receptor alpha constant-minus), when co-cultured with K562, RPMI8226, U266 or medium only. The data was collected at 3 days of co-culture. The co-culture flow cytometry study is described in Example 8.

FIG. 8B shows the results of a flow cytometry study comparing expansion capability of transgenic T cells (Donor 1) expressing the CAR BCMA bb2121 construct when co-cultured with K562, RPMI8226, U266 or medium only. The data was collected at 3 days of co-culture. The co-culture flow cytometry study is described in Example 8.

FIG. 8C shows the results of a flow cytometry study comparing expansion capability of transgenic T cells (Donor 1) expressing the CAR BCMA-2C5 construct when co-cultured with K562, RPMI8226, U266 or medium only. The data was collected at 3 days of co-culture. The co-culture flow cytometry study is described in Example 8.

FIG. 8D shows the results of a flow cytometry study comparing expansion capability of transgenic T cells (Donor 1) expressing the DAR BCMA-2C5 V2c construct when co-cultured with K562, RPMI8226, U266 or medium only. The data was collected at 3 days of co-culture. The co-culture flow cytometry study is described in Example 8.

FIG. 9 is a bar graph showing the fold-change in expansion of transgenic T cells, using data from FIGS. 8A-D, where the transgenic T cells express: the CAR bb2121 construct; the CAR BCMA-2C5 construct; or the DAR BCMA-2C5 V2c construct. The T cells were co-cultured with K562, RPMI8226 or U266 cell line. The data was collected at 3 days of co-culture. The fold-change expansion study is described in Example 8.

FIG. 10A shows the results of a flow cytometry study comparing expansion capability of negative control TRAC-minus T cell line (T-cell receptor alpha constant-minus), when co-cultured with K562, RPMI8226, U266 or medium only (the same data as presented in FIG. 8A). The data was collected at 3 days of co-culture. The co-culture study is described in Example 8.

FIG. 10B shows the results of a flow cytometry study comparing expansion capability of transgenic T cells (Donor 1) expressing the CAR BCMA bb2121 construct when co-cultured with K562, RPMI8226, U266 or medium only (the same data as presented in FIG. 10B). The data was collected at 3 days of co-culture. The co-culture study is described in Example 8.

FIG. 10C shows the results of a flow cytometry study comparing expansion capability of transgenic T cells (Donor 1) expressing the DAR BCMA-2C5 V2a construct when co-cultured with K562, RPMI8226, U266 or medium only. The data was collected at 3 days of co-culture. The co-culture study is described in Example 8.

FIG. 10D shows the results of a flow cytometry study comparing expansion capability of transgenic T cells (Donor 1) expressing the DAR BCMA-2C5 V2c construct when co-cultured with K562, RPMI8226, U266 or medium only. The data was collected at 3 days of co-culture. The co-culture study is described in Example 8.

FIG. 10E shows the results of a flow cytometry study comparing expansion capability of transgenic T cells (Donor 1) expressing the DAR BCMA-2C5 V3a construct when co-cultured with K562, RPMI8226, U266 or medium only. The data was collected at 3 days of co-culture. The co-culture study is described in Example 8.

FIG. 10F shows the results of a flow cytometry study comparing expansion capability of transgenic T cells (Donor 1) expressing the DAR BCMA-2C5 V3b construct when co-cultured with K562, RPMI8226, U266 or medium only. The data was collected at 3 days of co-culture. The co-culture study is described in Example 8.

FIG. 11 is a bar graph showing the fold-change in expansion of transgenic T cells, using the data from FIGS. 10A-E, where the transgenic T cells express: the CAR bb2121 construct; the DAR BCMA-2C5 V2c construct; the DAR BCMA-2C5 V3a construct; or the DAR BCMA-2C5 V3b construct. The T cells were co-cultured with K562, RPMI8226 or U266 cell line. The data was collected at 3 days of co-culture. The fold-change expansion study is described in Example 8.

FIG. 12 is a graph showing the percent cytotoxicity of T cells (Donor 1) expressing BCMA CAR, or BCMA DAR, on RPMI 8226 target cells. Line A designates the negative control TRAC-minus T cell line (T-cell receptor alpha constant-minus); Line B designates the DAR BCMA-2C5 V2c construct; Line C (dotted line) designates the CAR bb2121 construct; Line D designates the DAR BCMA-2C5 V3a construct; Line E designates the DAR BCMA-2C5 V2b construct; and Line F designates the CAR BCMA-2C5 construct. The cytotoxicity study is described in Example 6.

FIG. 13A shows the results of a flow cytometry study (at day 13) of the same BCMA-2C5 dimeric antigen receptor (DAR) constructs shown in FIG. 5A, comparing T cells (Donor 1) expressing three different versions of BCMA-2C5 dimeric antigen receptor (DAR) constructs. The negative control is a TRAC-minus T cell line from FIG. 5A. A comparison of transgenic cells expressing various DAR constructs is shown: DAR V2c construct; DAR V3a construct; and DAR V3b construct. The data was collected 13 days post-transfection. The transfection efficiency and expression level flow cytometry study is described in Example 5.

FIG. 13B shows the results of a flow cytometry study for detecting the fraction of central memory T cells in populations of anti-BCMA CAR T cells and DAR T cells, using the same cells described in FIG. 13A. The central memory T cell study is described in Example 9.

FIG. 13C shows the results of a flow cytometry study for detecting T cell exhaustion markers PD1 and TIM3 from anti-BCMA CAR T cells and DAR T cells, using the same cells described in FIG. 13A. The T cell exhaustion study is described in Example 10.

FIG. 14 shows the results of a flow cytometry study comparing transgenic T cells (Donor 2) expressing a BCMA chimeric antigen receptor (CAR) construct or two different versions of BCMA dimeric antigen receptor (DAR) constructs. The data was collected 11 days post-transfection and after 15 days expansion. The negative control is non-transgenic activated T cells (ATC). Another negative control is a TRAC-minus T cell line (T-cell receptor alpha constant-minus). The comparison includes transgenic T cells expressing: the CAR BCMA-2C5 construct; DAR BCMA-2C5 V2a construct; or DAR BCMA-2C5 V3a construct. The transfection efficiency and expression level flow cytometry study is described in Example 5.

FIG. 15 is a graph showing the percent cytotoxicity of transgenic T cells (Donor 2) expressing BCMA-2C5 CAR or BCMA-2C5 DAR constructs, on RPMI 8226 target cells. Line A designates the negative control TRAC-minus T cell line (T-cell receptor alpha constant-minus); Line B designates T cells expressing CAR BCMA-2C5 construct; Line C designates T cells expressing DAR BCMA-2C5 V3a construct; and Line D designates T cells expressing DAR BCMA-2C5 V2a construct. The cytotoxicity study is described in Example 6.

FIG. 16A shows the results of a flow cytometry study comparing expansion capability of negative control TRAC-minus T cell line (T-cell receptor alpha constant-minus) when co-cultured with K562, RPMI8226, Raji or medium only. The data was collected at 6 days of co-culture. The co-culture study is described in Example 8.

FIG. 16B shows the results of a flow cytometry study comparing expansion capability of non-transgenic activated T cells (ATC) (Donor 2) when co-cultured with K562, RPMI8226, Raji or medium only. The data was collected at 6 days of co-culture. The co-culture study is described in Example 8.

FIG. 16C shows the results of a flow cytometry study comparing expansion capability of transgenic T cells (Donor 2) expressing the CAR BCMA-2C5 construct when co-cultured with K562, RPMI8226, Raji or medium only. The data was collected at 6 days of co-culture. The co-culture study is described in Example 8.

FIG. 16D shows the results of a flow cytometry study comparing expansion capability of transgenic T cells (Donor 2) expressing the DAR BCMA-2C5 V2a construct (BBZ when co-cultured with K562, RPMI8226, Raji or medium only. The data was collected at 6 days of co-culture. The co-culture study is described in Example 8.

FIG. 16E shows the results of a flow cytometry study comparing expansion capability of transgenic T cells (Donor 2) expressing the DAR BCMA-2C5 V3a construct when co-cultured with K562, RPMI8226, Raji or medium only. The data was collected at 6 days of co-culture. The co-culture study is described in Example 8.

FIG. 17 is a bar graph showing the fold-change in expansion of transgenic T cells (Donor 2) expressing either a CAR construct or different DAR constructs having an antigen binding region from BCMA-2C5. The comparison includes T cells expressing: the CAR BCMA-2C5 construct; the DAR BCMA-2C5 V2a construct; and the DAR BCMA-2C5 V3a construct. The data was collected at 6 days of co-culture. The fold-change expansion study is described in Example 8.

FIG. 18A shows bioluminescent imaging of tumoricidal activity of BCMA DAR-expressing T cells in a xenograft mouse model (up to week 12 post-treatment). Mice harboring bioluminescent tumors were administered PBS buffer, TRAC-minus T cells, or transgenic T cells expressing a BCMA-2C5 DAR construct including DAR V2c, DAR V3b or DAR V3a. The xenograft mouse study is described in Example 11.

FIG. 18B is a graph showing the total flux (photons/sec) measured from the treated mice described in FIG. 18A. Line A designates DAR BCMA-2C5 V3a; Line B designates DAR BCMA-2C5 V3b; Line C designates DAR BCMA-2C5 V2c; Line D designates DAR BCMA-2C5 TRAC-minus T cells; and Line E designates PBS-treated mice. See Example 11.

FIG. 18C is a table listing the tumor growth inhibition indexes obtained from the mice described in FIG. 18A. The table lists data obtained up to week 8 post-treatment. See Example 11.

FIG. 18D is a graph showing the number of CD45-positive cells detected in blood samples from the mice described in FIG. 18A. The graph shows data obtained up to 12 weeks post-treatment. Line A designates PBS-treated mice; Line B designates DAR BCMA-2C5 V2c; Line C designates TRAC-minus T cells; Line D designates DAR BCMA-2C5 V3b; and Line E designates DAR BCMA-2C5 V3a. See Example 11.

FIG. 18E is a graph showing the number of DAR-positive cells detected in blood samples from the mice described in FIG. 18A. The graph shows data obtained up to 12 weeks post-treatment. Line A designates TRAC-minus T cells; Line B designates PBS-treated mice; Line C designates DAR BCMA-2C5 V2c; Line D designates DAR BCMA-2C5 V3b; and Line E designates DAR BCMA-2C5 V3a. See Example 11.

FIG. 18F is a graph showing the number of CD3-negative cells detected in blood samples from the mice described in FIG. 18A. The graph shows data obtained up to 12 weeks post-treatment. Line A designates PBS-treated mice; Line B designates DAR BCMA-2C5 V2c; Line C designates TRAC-minus T cells; Line D designates DAR BCMA-2C5 V3b; and Line E designates DAR BCMA-2C5 V3a. See Example 11.

FIG. 18G is a graph showing the number of CD3-positive cells detected in blood samples from the mice described in FIG. 18A. The graph shows data obtained up to 12 weeks post-treatment. Line A designates PBS-treated mice; Line B designates DAR BCMA-2C5 V2c; Line C designates TRAC-minus T cells; Line D designates DAR BCMA-2C5 V3a; and Line E designates DAR BCMA-2C5 V3b. See Example 11.

FIG. 18H is a graph showing the survival rate of the mice described in FIG. 18A. Line A designates PBS-treated mice; Line B designates TRAC-minus T cells; Line C designates DAR BCMA-2C5 V2c; Line D designates DAR BCMA-2C5 V3b; and Line E designates DAR BCMA-2C5 V3a. See Example 11.

FIG. 19A shows bioluminescent imaging of tumoricidal activity of BCMA DAR-expressing T cells in a xenograft mouse model (up to week 12 post-treatment). Mice harboring bioluminescent RPMI8226 tumors were administered PBS buffer, TRAC-minus T cells, or one of three different doses of transgenic T cells expressing a DAR BCMA-2C5 V3a construct. The xenograft mouse study is described in Example 12.

FIG. 19B is a graph showing the total flux (photons/sec) measured from the treated mice described in FIG. 19A. The graphs shows data obtained up to day 76 post-treatment. Line A designates mice administered with 6×10⁶ cells of DAR BCMA-2C5 V3a; Line B designates mice administered with 1.2×10⁶ cells of DAR BCMA-2C5 V3a; Line C designates mice administered with 2.4×10⁵ cells of DAR BCMA-2C5 V3a; Line D designates mice administered TRAC-minus T cells; and Line E designates mice administered PBS. See example 12.

FIG. 19C is a table listing the tumor growth inhibition indexes obtained from the mice described in FIG. 19A. The table lists data obtained up to week 7 post-treatment. See example 12.

FIG. 19D is a graph showing the number of CD45-positive cells detected in blood samples from the mice described in FIG. 19A. The graph shows data obtained up to day 65 post-treatment. Line A designates PBS-treated mice; Line B designates TRAC-minus T cells; Line C designates mice administered 2.4×10⁵ of DAR BCMA-2C5 V3a; Line D designates mice administered 1.2×10⁶ of DAR BCMA-2C5 V3a; and Line E designates mice administered 6×10⁶ of DAR BCMA-2C5 V3a. See example 12.

FIG. 19E is a graph showing the number of DAR-positive cells detected in blood samples from the mice described in FIG. 19A. The graph shows data obtained up to day 65 post-treatment. Line A designates PBS-treated mice; Line B designates TRAC-minus T cells; Line C designates mice administered 2.4×10⁵ of DAR BCMA-2C5 V3a; Line D designates mice administered 1.2×10⁶ of DAR BCMA-2C5 V3a; and Line E designates mice administered 6×10⁶ of DAR BCMA-2C5 V3a. See example 12.

FIG. 19F is a graph showing the number of CD3-negative cells detected in blood samples from the mice described in FIG. 19A. The graph shows data obtained up to day 65 post-treatment. Line A designates PBS-treated mice; Line B designates TRAC-minus T cells; Line C designates mice administered 2.4×10⁵ of DAR BCMA-2C5 V3a; Line D designates mice administered 1.2×10⁶ of DAR BCMA-2C5 V3a; and Line E designates mice administered 6×10⁶ of DAR BCMA-2C5 V3a. See example 12.

FIG. 19G is a graph showing the number of CD3-positive cells detected in blood samples from the mice described in FIG. 19A. The graph shows data obtained up to day 65 post-treatment. Line A designates PBS-treated mice; Line B designates mice administered 2.4×10⁵ of DAR BCMA-2C5 V3a; Line C designates mice administered 1.2×10⁶ of DAR BCMA-2C5 V3a; Line D designates mice administered designates TRAC-minus T cells; and Line E designates mice administered 6×10⁶ of DAR BCMA-2C5 V3a. See example 12.

FIG. 19H is a graph showing the survival rate of the mice described in FIG. 19A. Line A designates mice administered PBS; Line B designates mice administered TRAC-minus T cells; Line C designates mice administered 1.2×10⁶ of DAR BCMA-2C5 V3a; Line D designates mice administered 1.2×10⁶ of DAR BCMA-2C5 V3a; and Line E designates mice administered 6×10⁶ of DAR BCMA-2C5 V3a. See example 12.

FIG. 20A shows bioluminescent imaging of tumoricidal activity of BCMA DAR-expressing T cells in a xenograft mouse model, where the mice described in FIG. 19A were re-challenged with RPMI8226 bioluminescent tumors but were not administered additional DAR T cells. The bioluminescent data shows up to week 7 post-re-challenge. The xenograft mouse study is described in Example 13.

FIG. 20B is a graph showing the number of CD45-positive cells detected in blood samples from the tumor re-challenged mice described in FIG. 20A. The graph shows data obtained up to day 65 post-treatment. Line A designates mice re-challenged with RPMI tumor cells; and Line B designates mice re-challenged with PBS.

FIG. 20C is a graph showing the number of DAR-positive cells detected in blood samples from the tumor re-challenged mice described in FIG. 20A. The graph shows data obtained up to day 65 post-treatment. Line A designates mice re-challenged with RPMI tumor cells; and Line B designates mice re-challenged with PBS.

FIG. 21 shows the amino acid sequence of wild type human BCMA antigen, mutant-1 human BCMA antigen, mutant-2 human BCMA antigen, human APRIL antigen and human BAFF antigen.

FIG. 22 shows the amino acid sequence of anti-BCMA-2C5 heavy chain variable region, heavy chain constant region, light chain variable region and light chain constant region.

FIG. 23 shows the amino acid sequence of anti-BCMA heavy chain variable and light chain variable regions of anti-BCMA-2E1, -BC4C9 and -BC5C4.

FIG. 24 shows the amino acid sequence of anti-BCMA heavy chain variable and light chain variable regions of anti-BCMA-BC6G8, -2D11 and -2G2.

FIG. 25 shows the amino acid sequence of anti-BCMA heavy chain variable and light chain variable regions of anti-BCMA-2D8 and -2E8.

FIG. 26 shows the amino acid sequence of anti-BCMA heavy chain variable region, heavy chain constant region, light chain variable region and light chain constant region, of anti-BCMA-bb2121.

FIG. 27 shows the amino acid sequence of CAR GS linker, CAR bb2121 linker, CD8 hinge region, CD28 hinge region, CD8 and CD28 hinge region, CD28 transmembrane region, CD8 transmembrane region, 4-1BB transmembrane region and CD3zeta transmembrane region.

FIG. 28 shows the amino acid sequences of intracellular regions for 4-1BB, CD28, OX40, CD3zeta (ITAM 1, 2 and 3), CD3zeta ITAM 1, CD3zeta ITAM 2 and CD3zeta ITAM 3.

FIG. 29 shows the amino acid sequences of CAR intracellular domain 28Z, and of DAR intracellular domains for V1, V2a, V2b, V2c, V3a, V3b and V4.

FIG. 30 shows the amino acid sequences of DAR intracellular domains for V3c, V2c-alt and V3b-alt.

FIG. 31 shows the amino acid sequence of heavy and light chain leader sequences, and four different self-cleaving sequences including T2A, P2A, E2A and F2A.

FIG. 32 shows the amino acid sequence of CAR 28Z BCMA-2C5 and BCMA-bb2121.

FIG. 33 shows the amino acid sequence of precursor, first polypeptide and second polypeptide for DAR V1 BCMA-2C5.

FIG. 34 shows the amino acid sequence of precursor, first polypeptide and second polypeptide for DAR V2a BCMA-2C5.

FIG. 35 shows the amino acid sequence of precursor, first polypeptide and second polypeptide for DAR V2b BCMA-2C5.

FIG. 36 shows the amino acid sequence of precursor, first polypeptide and second polypeptide for DAR V2c BCMA-2C5.

FIG. 37 shows the amino acid sequence of precursor, first polypeptide and second polypeptide for DAR V3a BCMA-2C5.

FIG. 38 shows the amino acid sequence of precursor, first polypeptide and second polypeptide for DAR V3b BCMA-2C5.

FIG. 39 shows the amino acid sequence of precursor, first polypeptide and second polypeptide for DAR V4 BCMA-2C5.

FIG. 40 shows the amino acid sequence of precursor, first polypeptide and second polypeptide for DAR V2a BCMA-bb2121.

### DETAILED DESCRIPTION

### Definitions

Unless defined otherwise, technical and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art. The methods and techniques provided herein are generally performed according to conventional procedures well known in the art and as described in various general and more specific references that are cited and discussed herein unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebaeck (ed) Antibody Engineering, 2nd Edition Freeman and Company, N Y, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N.Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N.Y., 1986, and Kohler and Milstein Nature 256: 495-497, 1975. All of the references cited herein are incorporated herein by reference in their entireties. Enzymatic reactions and enrichment/purification techniques are also well known and are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are well known and commonly used in the art. Standard techniques can be used for chemical syntheses, chemical analyses, pharmaceutical preparation, formulation, and delivery, and treatment of patients.

The headings provided herein are not limitations of the various aspects of the disclosure, which aspects can be understood by reference to the specification as a whole.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

The terms “peptide”, “polypeptide”, “polypeptide chain” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. Polypeptides also include precursor molecules and mature molecule. Precursor molecules include those that have not yet been subjected to cleavage, for example cleavage by a secretory signal peptide or by non-enzymatic cleavage at certain amino acid residue. Polypeptides in include mature molecules that have undergone cleavage. These terms encompass native proteins, recombinant proteins and artificial proteins, protein fragments and polypeptide analogs (such as muteins, variants, chimeric proteins and fusion proteins) of a protein sequence as well as post-translationally, or otherwise covalently or non-covalently, modified proteins. Two or more polypeptides (e.g., 2-6 or more polypeptide chains) can associate with each other, via covalent and/or non-covalent association, to form a polypeptide complex. Association of the polypeptide chains can also include peptide folding. Thus, a polypeptide complex can be dimeric, trimeric, tetrameric, or higher order complexes depending on the number of polypeptide chains that form the complex. Dimeric antigen receptors (DAR) comprising two polypeptide chains are described herein.

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids include DNA molecules (cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. Nucleic acid molecule can be single-stranded or double-stranded. In one embodiment, the nucleic acid molecules of the disclosure comprise a contiguous open reading frame encoding a dimeric antigen receptor (DAR) construct, or a fragment or scFv, derivative, mutein, or variant thereof. In one embodiment, nucleic acids comprise one type of polynucleotide or a mixture of two or more different types of polynucleotides. Nucleic acids encoding dimeric antigen receptors (DAR) or antigen-binding portions thereof, are described herein. With respect to embodiments involving a first nucleic acid (e.g., encoding a first polypeptide) and a second nucleic acid (e.g., encoding a second polypeptide), the first nucleic acid and second nucleic acid may be provided either as separate molecules or within the same continuous molecule (e.g., a plasmid or other construct containing first and second coding sequences).

The term “recover” or “recovery” or “recovering”, and other related terms, refers to obtaining a protein (e.g., a DAR or a precursor or an antigen binding portion thereof), from host cell culture medium or from host cell lysate or from the host cell membrane. In one embodiment, the protein is expressed by the host cell as a recombinant protein fused to a secretion signal peptide (leader peptide sequence) sequence which mediates secretion of the expressed protein from a host cell (e.g., from a mammalian host cell). The secreted protein can be recovered from the host cell medium. In one embodiment, the protein is expressed by the host cell as a recombinant protein that lacks a secretion signal peptide sequence which can be recovered from the host cell lysate. In one embodiment, the protein is expressed by the host cell as a membrane-bound protein which can be recovered using a detergent to release the expressed protein from the host cell membrane. In one embodiment, irrespective of the method used to recover the protein, the protein can be subjected to procedures that remove cellular debris from the recovered protein. For example, the recovered protein can be subjected to chromatography, gel electrophoresis and/or dialysis. In one embodiment, the chromatography comprises any one or any combination or two or more procedures including affinity chromatography, hydroxyapatite chromatography, ion-exchange chromatography, reverse phase chromatography and/or chromatography on silica. In one embodiment, affinity chromatography comprises protein A or G (cell wall components from Staphylococcus aureus).

The term “isolated” refers to a protein (e.g., a DAR or precursor or an antigen binding portion thereof) or polynucleotide that is substantially free of other cellular material. A protein may be rendered substantially free of naturally associated components (or components associated with a cellular expression system or chemical synthesis methods used to produce the DAR) by isolation, using protein purification techniques well known in the art. The term isolated also refers in some embodiment to protein or polynucleotides that are substantially free of other molecules of the same species, for example other protein or polynucleotides having different amino acid or nucleotide sequences, respectively. The purity or homogeneity of the desired molecule can be assayed using techniques well known in the art, including low resolution methods such as gel electrophoresis and high resolution methods such as HPLC or mass spectrometry. In one embodiment, isolated precursor polypeptides, and first and second polypeptide chains, of the dimeric antigen receptor (DAR) or antigen-binding portions thereof, of the present disclosure are isolated.

Antibodies, including the dimeric antigen receptors (DAR) described herein can be obtained from sources such as serum or plasma that contain immunoglobulins having varied antigenic specificity. If such antibodies are subjected to affinity purification, they can be enriched for a particular antigenic specificity. Such enriched preparations of antibodies usually are made of less than about 10% antibody having specific binding activity for the particular antigen. Subjecting these preparations to several rounds of affinity purification can increase the proportion of antibody having specific binding activity for the antigen. Antibodies prepared in this manner are often referred to as “monospecific.” Monospecific antibody preparations can be made up of about 10%, 20%, 30%, 40%, 50%, 60%, 70%, 75%, 80%, 85%, 90%, 95%, 97%, 99%, or 99.9% antibody having specific binding activity for the particular antigen. Antibodies can be produced using recombinant nucleic acid technology as described below.

The term “leader sequence” or “leader peptide” or “peptide signal sequence” or “signal peptide” or “secretion signal peptide” refers to a peptide sequence that is located at the N-terminus of a polypeptide. A leader sequence directs a polypeptide chain to a cellular secretory pathway and can direct integration and anchoring of the polypeptide into the lipid bilayer of the cellular membrane. Typically, a leader sequence is about 10-50 amino acids in length. A leader sequence can direct transport of a precursor polypeptide from the cytosol to the endoplasmic reticulum. In one embodiment, a leader sequence includes signal sequences comprising CD8a, CD28 or CD16 leader sequences. In one embodiment, the signal sequence comprises a mammalian sequence, including for example mouse or human Ig gamma secretion signal peptide. In one embodiment, a leader sequence comprises a mouse Ig gamma leader peptide sequence MEWSWVFLFFLSVTTGVHS (SEQ ID NO: 90).

An “antigen binding protein” and related terms used herein refers to a protein comprising a portion that binds to an antigen and, optionally, a scaffold or framework portion that allows the antigen binding portion to adopt a conformation that promotes binding of the antigen binding protein to the antigen. Examples of antigen binding proteins include dimeric antigen receptors (DARs), antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold. Antigen binding proteins comprising dimeric antigen receptors (DAR) are described herein.

An antigen binding protein can have, for example, the structure of an immunoglobulin. In one embodiment, an “immunoglobulin” refers to a tetrameric molecule composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The heavy and/or light chains may or may not include a leader sequence for secretion. The variable regions of each light/heavy chain pair form the antibody binding site such that an intact immunoglobulin has two antigen binding sites. In one embodiment, an antigen binding protein can be a synthetic molecule having a structure that differs from a tetrameric immunoglobulin molecule but still binds a target antigen or binds two or more target antigens. For example, a synthetic antigen binding protein can comprise antibody fragments, 1-6 or more polypeptide chains, asymmetrical assemblies of polypeptides, or other synthetic molecules. Antigen binding proteins having dimeric antigen receptor (DAR) structures with immunoglobulin-like properties that bind specifically to a target antigen (e.g., BCMA antigen) are described herein.

The variable regions of immunoglobulin chains exhibit the same general structure of relatively conserved framework regions (FR) joined by three hypervariable regions, also called complementarity determining regions or CDRs. From N-terminus to C-terminus, both light and heavy chains comprise the segments FR1, CDR1, FR2, CDR2, FR3, CDR3 and FR4.

One or more CDRs may be incorporated into a molecule either covalently or noncovalently to make it an antigen binding protein. An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest.

The assignment of amino acids to each domain is in accordance with the definitions of Kabat et al. in Sequences of Proteins of Immunological Interest, 5^{th} Ed., US Dept. of Health and Human Services, PHS, NIH, NIH Publication no. 91-3242, 1991 (“Kabat numbering”). Other numbering systems for the amino acids in immunoglobulin chains include IMGT® (international ImMunoGeneTics information system; Lefranc et al, Dev. Comp. Immunol. 29:185-203; 2005) and AHo (Honegger and Pluckthun, J. Mol. Biol. 309(3):657-670; 2001); Chothia (Al-Lazikani et al., 1997 Journal of Molecular Biology 273:927-948; Contact (Maccallum et al., 1996 Journal of Molecular Biology 262:732-745, and Aho (Honegger and Pluckthun 2001 Journal of Molecular Biology 309:657-670.

An “antibody” and “antibodies” and related terms used herein refers to an intact immunoglobulin or to an antigen binding portion thereof that binds specifically to an antigen. Antigen binding portions may be produced by recombinant DNA techniques or by enzymatic or chemical cleavage of intact antibodies. Antigen binding portions include, inter alia, Fab, Fab′, F(ab′)₂, Fv, domain antibodies (dAbs), and complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen binding to the polypeptide.

Antibodies include recombinantly produced antibodies and antigen binding portions. Antibodies include non-human, chimeric, humanized and fully human antibodies. Antibodies include monospecific, multispecific (e.g., bispecific, trispecific and higher order specificities). Antibodies include tetrameric antibodies, light chain monomers, heavy chain monomers, light chain dimers, heavy chain dimers. Antibodies include F(ab′)₂ fragments, Fab′ fragments and Fab fragments. Antibodies include single domain antibodies, monovalent antibodies, single chain antibodies, single chain variable fragment (scFv), camelized antibodies, affibodies, disulfide-linked Fvs (sdFv), anti-idiotypic antibodies (anti-Id), minibodies. Antibodies include monoclonal and polyclonal populations. Antibodies-like molecules comprising dimeric antigen receptors (DAR) are described herein.

An “antigen binding domain,” “antigen binding region,” or “antigen binding site” and other related terms used herein refer to a portion of an antigen binding protein that contains amino acid residues (or other moieties) that interact with an antigen and contribute to the antigen binding protein's specificity and affinity for the antigen. For an antibody that specifically binds to its antigen, this will include at least part of at least one of its CDR domains. Dimeric antigen receptors (DAR) having antibody heavy chain variable regions and antibody light chain variable regions that form antigen binding domains are described herein.

The terms “specific binding”, “specifically binds” or “specifically binding” and other related terms, as used herein in the context of an antibody or antigen binding protein or antibody fragment, refer to non-covalent or covalent preferential binding to an antigen relative to other molecules or moieties (e.g., an antibody specifically binds to a particular antigen relative to other available antigens). In one embodiment, an antibody specifically binds to a target antigen if it binds to the antigen with a dissociation constant K_{D} of 10⁻⁵ M or less, or 10⁻⁶ M or less, or 10⁻⁷ M or less, or 10⁻⁸ M or less, or 10⁻⁹ M or less, or 10⁻¹⁰ M or less, or 10⁻¹¹ M or less. In one embodiment, dimeric antigen receptors (DAR) that bind specifically to their target antigen (e.g., BCMA antigen) are described herein.

In one embodiment, binding specificity of an antibody or antigen binding protein or antibody fragment can be measure by ELISA, radioimmune assay (RIA), electrochemiluminescence assays (ECL), immunoradiometric assay (IRMA), or enzyme immune assay (EIA).

In one embodiment, a dissociation constant (K_{D}) can be measured using a BIACORE surface plasmon resonance (SPR) assay. Surface plasmon resonance refers to an optical phenomenon that allows for the analysis of real-time interactions by detection of alterations in protein concentrations within a biosensor matrix, for example using the BIACORE system (Biacore Life Sciences division of GE Healthcare, Piscataway, N.J.).

An “epitope” and related terms as used herein refers to a portion of an antigen that is bound by an antigen binding protein (e.g., by an antibody or an antigen binding portion thereof). An epitope can comprise portions of two or more antigens that are bound by an antigen binding protein. An epitope can comprise non-contiguous portions of an antigen or of two or more antigens (e.g., amino acid residues that are not contiguous in an antigen's primary sequence but that, in the context of the antigen's tertiary and quaternary structure, are near enough to each other to be bound by an antigen binding protein). Generally, the variable regions, particularly the CDRs, of an antibody interact with the epitope. In one embodiment, dimeric antigen receptors (DAR) or antigen-binding portions thereof that bind an epitope of BCMA antigen are described herein.

An “antibody fragment”, “antibody portion”, “antigen-binding fragment of an antibody”, or “antigen-binding portion of an antibody” and other related terms used herein refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; Fd; and Fv fragments, as well as dAb; diabodies; linear antibodies; single-chain antibody molecules (e.g. scFv); polypeptides that contain at least a portion of an antibody that is sufficient to confer specific antigen binding to the polypeptide. Antigen binding portions of an antibody may be produced by recombinant DNA techniques or by enzymatic or chemical cleavage of intact antibodies. Antigen binding portions include, inter alia, Fab, Fab′, F(ab′)2, Fv, domain antibodies (dAbs), and complementarity determining region (CDR) fragments, chimeric antibodies, diabodies, triabodies, tetrabodies, and polypeptides that contain at least a portion of an immunoglobulin that is sufficient to confer antigen binding properties to the antibody fragment. In one embodiment, dimeric antigen receptors comprising a Fab fragment joined to a hinge, transmembrane and intracellular regions are described herein.

The terms “Fab”, “Fab fragment” and other related terms refers to a monovalent fragment comprising a variable light chain region (V_{L}), constant light chain region (C_{L}), variable heavy chain region (V_{H}), and first constant region (C_{H1}). A Fab is capable of binding an antigen. An F(ab′)₂ fragment is a bivalent fragment comprising two Fab fragments linked by a disulfide bridge at the hinge region. A F(Ab′)₂ has antigen binding capability. An Fd fragment comprises V_{H} and C_{H1} regions. An Fv fragment comprises V_{L} and V_{H} regions. An Fv can bind an antigen. A dAb fragment has a V_{H} domain, a V_{L} domain, or an antigen-binding fragment of a V_{H} or V_{L} domain (U.S. Pat. Nos. 6,846,634 and 6,696,245; U.S. published Application Nos. 2002/02512, 2004/0202995, 2004/0038291, 2004/0009507, 2003/0039958; and Ward et al., Nature 341:544-546, 1989). In one embodiment, dimeric antigen receptors comprising a Fab fragment joined to a hinge, transmembrane and intracellular regions are described herein.

A single-chain antibody (scFv) is an antibody in which a V_{L} and a V_{H} region are joined via a linker (e.g., a synthetic sequence of amino acid residues) to form a continuous protein chain. In one embodiment, the linker is long enough to allow the protein chain to fold back on itself and form a monovalent antigen binding site (see, e.g., Bird et al., 1988, Science 242:423-26 and Huston et al., 1988, Proc. Natl. Acad. Sci. USA 85:5879-83).

Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., 1993, Proc. Natl. Acad. Sci. USA 90:6444-48, and Poljak et al., 1994, Structure 2:1121-23). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different. Diabody, tribody and tetrabody constructs can be prepared using antigen binding portions from any of the dimeric antigen receptors (DAR) described herein.

The term “human antibody” refers to antibodies that have one or more variable and constant regions derived from human immunoglobulin sequences. In one embodiment, all of the variable and constant domains are derived from human immunoglobulin sequences (e.g., a fully human antibody). These antibodies may be prepared in a variety of ways, examples of which are described below, including through recombinant methodologies or through immunization with an antigen of interest of a mouse that is genetically modified to express antibodies derived from human heavy and/or light chain-encoding genes. Dimeric antigen receptors (DAR) comprising fully human antibody heavy chain variable region and fully human antibody light chain variable regions are described herein.

A “humanized” antibody refers to an antibody having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” and related terms used herein refers to an antibody that contains one or more regions from a first antibody and one or more regions from one or more other antibodies. In one embodiment, one or more of the CDRs are derived from a human antibody. In another embodiment, all of the CDRs are derived from a human antibody. In another embodiment, the CDRs from more than one human antibody are mixed and matched in a chimeric antibody. For instance, a chimeric antibody may comprise a CDR1 from the light chain of a first human antibody, a CDR2 and a CDR3 from the light chain of a second human antibody, and the CDRs from the heavy chain from a third antibody. In another example, the CDRs originate from different species such as human and mouse, or human and rabbit, or human and goat. One skilled in the art will appreciate that other combinations are possible.

Further, the framework regions may be derived from one of the same antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind a target antigen). Chimeric antibodies can be prepared from portions of any of the dimeric antigen receptor (DAR) antigen-binding portions thereof are described herein.

As used herein, the term “variant” polypeptides and “variants” of polypeptides refers to a polypeptide comprising an amino acid sequence with one or more amino acid residues inserted into, deleted from and/or substituted into the amino acid sequence relative to a reference polypeptide sequence. Polypeptide variants include fusion proteins. In the same manner, a variant polynucleotide comprises a nucleotide sequence with one or more nucleotides inserted into, deleted from and/or substituted into the nucleotide sequence relative to another polynucleotide sequence. Polynucleotide variants include fusion polynucleotides.

As used herein, the term “derivative” of a polypeptide is a polypeptide (e.g., an antibody) that has been chemically modified, e.g., via conjugation to another chemical moiety such as, for example, polyethylene glycol, albumin (e.g., human serum albumin), phosphorylation, and glycosylation. Unless otherwise indicated, the term “antibody” includes, in addition to antibodies comprising full-length heavy chains and full-length light chains, derivatives, variants, fragments, and muteins thereof, examples of which are described below.

The term “hinge” refers to an amino acid segment that is generally found between two domains of a protein and may allow for flexibility of the overall construct and movement of one or both of the domains relative to one another. Structurally, a hinge region comprises from about 10 to about 100 amino acids, e.g., from about 15 to about 75 amino acids, from about 20 to about 50 amino acids, or from about 30 to about 60 amino acids. In one embodiment, the hinge region is 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 amino acids in length. The hinge region can be derived from is a hinge region of a naturally-occurring protein, such as a CD8 hinge region or a fragment thereof, a CD8a hinge region, or a fragment thereof, a hinge region of an antibody (e.g., IgG, IgA, IgM, IgE, or IgD antibodies), or a hinge region that joins the constant domains CH1 and CH2 of an antibody. The hinge region can be derived from an antibody and may or may not comprise one or more constant regions of the antibody, or the hinge region comprises the hinge region of an antibody and the CH3 constant region of the antibody, or the hinge region comprises the hinge region of an antibody and the CH2 and CH3 constant regions of the antibody, or the hinge region is a non-naturally occurring peptide, or the hinge region is disposed between the C-terminus of the scFv and the N-terminus of the transmembrane domain. In one embodiment, the hinge region comprises any one or any combination of two or more regions comprising an upper, core or lower hinge sequences from an IgG1, IgG2, IgG3 or IgG4 immunoglobulin molecule. In one embodiment, the hinge region comprises an IgG1 upper hinge sequence EPKSCDKTHT (SEQ ID NO: 91). In one embodiment, the hinge region comprises an IgG1 core hinge sequence CPXC, wherein X is P, R or S (SEQ ID NO: 92). In one embodiment, the hinge region comprises a lower hinge/CH2 sequence PAPELLGGP (SEQ ID NO: 93). In one embodiment, the hinge is joined to an Fc region (CH2) having the amino acid sequence SVFLFPPKPKDT (SEQ ID NO: 94). In one embodiment, the hinge region includes the amino acid sequence of an upper, core and lower hinge and comprises EPKSCDKTHTCPPCPAP ELLGGP (SEQ ID NO: 95). In one embodiment, the hinge region comprises one, two, three or more cysteines that can form at least one, two, three or more interchain disulfide bonds.

The term “Fc” or “Fc region” as used herein refers to the portion of an antibody heavy chain constant region beginning in or after the hinge region and ending at the C-terminus of the heavy chain. The Fc region comprises at least a portion of the CH2 and CH3 regions, and may or may not include a portion of the hinge region. An Fc region can bind Fc cell surface receptors and some proteins of the immune complement system. An Fc region exhibits effector function, including any one or any combination of two or more activities including complement-dependent cytotoxicity (CDC), antibody-dependent cell-mediated cytotoxicity (ADCC), antibody-dependent phagocytosis (ADP), opsonization and/or cell binding. In one embodiment, the Fc region can include a mutation that increases or decreases any one or any combination of these functions. An Fc region can bind an Fc receptor, including FcγRI (e.g., CD64), FcγRII (e.g., CD32) and/or FcγRIII (e.g., CD16a). An Fc region can bind a complement component C1q. In one embodiment, the Fc domain comprises a LALA-PG mutation (e.g., equivalent to L234A, L235A, P329G) which reduces effector function. In one embodiment, the Fc domain mediates serum half-life of the protein complex, and a mutation in the Fc domain can increase or decrease the serum half-life of the protein complex. In one embodiment, the Fc domain affects thermal stability of the protein complex, and mutation in the Fc domain can increase or decrease the thermal stability of the protein complex.

The term “labeled” or related terms as used herein with respect to a polypeptide refers to joinder thereof to a detectable label or moiety for detection. Exemplary detectable labels or moieties include radioactive, colorimetric, antigenic, enzymatic labels/moieties, a detectable bead (such as a magnetic or electrodense (e.g., gold) bead), biotin, streptavidin or protein A. A variety of labels can be employed, including, but not limited to, radionuclides, fluorescers, enzymes, enzyme substrates, enzyme cofactors, enzyme inhibitors and ligands (e.g., biotin, haptens). Any of the dimeric antigen receptors (DAR) or antigen-binding portions thereof that described herein can be unlabeled or can be joined to a detectable label or detectable moiety.

The “percent identity” or “percent homology” and related terms used herein refers to a quantitative measurement of the similarity between two polypeptide or between two polynucleotide sequences. The percent identity between two polypeptide sequences is a function of the number of identical amino acids at aligned positions that are shared between the two polypeptide sequences, taking into account the number of gaps, and the length of each gap, which may need to be introduced to optimize alignment of the two polypeptide sequences. In a similar manner, the percent identity between two polynucleotide sequences is a function of the number of identical nucleotides at aligned positions that are shared between the two polynucleotide sequences, taking into account the number of gaps, and the length of each gap, which may need to be introduced to optimize alignment of the two polynucleotide sequences. A comparison of the sequences and determination of the percent identity between two polypeptide sequences, or between two polynucleotide sequences, may be accomplished using a mathematical algorithm. For example, the “percent identity” or “percent homology” of two polypeptide or two polynucleotide sequences may be determined by comparing the sequences using the GAP computer program (a part of the GCG Wisconsin Package, version 10.3 (Accelrys, San Diego, Calif.)) using its default parameters. Expressions such as “comprises a sequence with at least X % identity to Y” with respect to a test sequence mean that, when aligned to sequence Y as described above, the test sequence comprises residues identical to at least X % of the residues of Y.

In one embodiment, the amino acid sequence of a test construct (e.g., DAR) may be similar but not necessarily identical to any of the amino acid sequences of the polypeptides that make up a given dimeric antigen receptor (DAR) or antigen-binding portions thereof that are described herein. The similarities between the test construct and the polypeptides can be at least 95%, or at or at least 96% identical, or at least 97% identical, or at least 98% identical, or at least 99% identical, to any of the polypeptides that make up the dimeric antigen receptor (DAR) or antigen-binding portions thereof that are described herein. In one embodiment, similar polypeptides can contain amino acid substitutions within a heavy and/or light chain. In one embodiment, the amino acid substitutions comprise one or more conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference in its entirety. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

The term “Chimeric Antigen Receptor” or “CAR” refers to a single chain fusion protein comprising an extracellular antigen-binding protein that is fused to an intracellular signaling domain. The CAR extracellular binding domain is a single chain variable fragment (scFv or sFv) derived from fusing the variable heavy and light regions of a monoclonal antibody, such as a human monoclonal antibody. In one embodiment, a CAR comprises (i) an antigen binding protein comprising a heavy chain variable (VH) domain and a light chain variable (VL) domain wherein the VH and VL domains are joined together by a peptide linker; (ii) a hinge domain, (iii) a transmembrane domain; and (iv) an intracellular domain comprising an intracellular signaling sequence. The disclosed constructs are DARs which are distinct from CARs in that DARs do not use a single chain antibody for targeting but instead use separate heavy and light chain variable domain regions.

A “vector” and related terms used herein refers to a nucleic acid molecule (e.g., DNA or RNA) which can be operably linked to foreign genetic material (e.g., nucleic acid transgene). Vectors can be used as a vehicle to introduce foreign genetic material into a cell (e.g., host cell). Vectors can include at least one restriction endonuclease recognition sequence for insertion of the transgene into the vector. Vectors can include at least one gene sequence that confers antibiotic resistance or a selectable characteristic to aid in selection of host cells that harbor a vector-transgene construct. Vectors can be single-stranded or double-stranded nucleic acid molecules. Vectors can be linear or circular nucleic acid molecules. A donor nucleic acid used for gene editing methods employing zinc finger nuclease, TALEN or CRISPR/Cas can be a type of a vector. One type of vector is a “plasmid,” which refers to a linear or circular double stranded extrachromosomal DNA molecule which can be linked to a transgene, and is capable of replicating in a host cell, and transcribing and/or translating the transgene. A viral vector typically contains viral RNA or DNA backbone sequences which can be linked to the transgene. The viral backbone sequences can be modified to disable infection but retain insertion of the viral backbone and the co-linked transgene into a host cell genome. Examples of viral vectors include retroviral, lentiviral, adenoviral, adeno-associated, baculoviral, papovaviral, vaccinia viral, herpes simplex viral and Epstein Barr viral vectors. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome.

An “expression vector” is a type of vector that can contain one or more regulatory sequences, such as inducible and/or constitutive promoters and enhancers. Expression vectors can include ribosomal binding sites and/or polyadenylation sites. Expression vectors can include one or more origin of replication sequence. Regulatory sequences direct transcription, or transcription and translation, of a transgene linked to the expression vector which is transduced into a host cell. The regulatory sequence(s) can control the level, timing and/or location of expression of the transgene. The regulatory sequence can, for example, exert its effects directly on the transgene, or through the action of one or more other molecules (e.g., polypeptides that bind to the regulatory sequence and/or the nucleic acid). Regulatory sequences can be part of a vector. Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-3606. An expression vector can comprise nucleic acids that encode at least a portion of any of the dimeric antigen receptors (DAR) or antigen-binding portions thereof that are described herein.

A transgene is “operably linked” to a vector when there is linkage between the transgene and the vector to permit functioning or expression of the transgene sequences contained in the vector. In one embodiment, a transgene is “operably linked” to a regulatory sequence when the regulatory sequence affects the expression (e.g., the level, timing, or location of expression) of the transgene.

The terms “transfected” or “transformed” or “transduced” or other related terms used herein refer to a process by which exogenous nucleic acid (e.g., transgene) is transferred or introduced into a host cell. A “transfected” or “transformed” or “transduced” host cell is one which has been introduced with exogenous nucleic acid (transgene). The host cell includes the primary subject cell and its progeny. Exogenous nucleic acids encoding at least a portion of any of the dimeric antigen receptors (DAR) or antigen-binding portions thereof that are described herein can be introduced into a host cell. Expression vectors comprising at least a portion of any of the dimeric antigen receptors (DAR) or antigen-binding portions thereof that are described herein can be introduced into a host cell, and the host cell can express polypeptides comprising at least a portion of the dimeric antigen receptor (DAR) or antigen-binding portions thereof that are described herein.

The terms “host cell” or “or a population of host cells” or related terms as used herein refer to a cell (or a population thereof) into which foreign (exogenous or transgene) nucleic acids have been introduced. The foreign nucleic acids can include an expression vector operably linked to a transgene, and the host cell can be used to express the nucleic acid and/or polypeptide encoded by the foreign nucleic acid (transgene). A host cell (or a population thereof) can be a cultured cell or can be extracted from a subject. The host cell (or a population thereof) includes the primary subject cell and its progeny without any regard for the number of passages. The host cell (or a population thereof) includes immortalized cell lines. Progeny cells may or may not harbor identical genetic material compared to the parent cell. Host cells encompass progeny cells. In one embodiment, a host cell describes any cell (including its progeny) that has been modified, transfected, transduced, transformed, and/or manipulated in any way to express an antibody, as disclosed herein. In one example, the host cell (or population thereof) can be introduced with an expression vector operably linked to a nucleic acid encoding the desired antibody, or an antigen binding portion thereof, described herein. Host cells and populations thereof can harbor an expression vector that is stably integrated into the host's genome, or can harbor an extrachromosomal expression vector. In one embodiment, host cells and populations thereof can harbor an extrachromosomal vector that is present after several cell divisions or is present transiently and is lost after several cell divisions.

Transgenic host cells can be prepared using non-viral methods, including well-known designer nucleases including zinc finger nucleases, TALENS, maganucleases, or by gene editing using CRISPR/Cas. A transgene can be introduced into a host cell's genome using a zinc finger nuclease. A zinc finger nuclease includes a pair of chimeric proteins each containing a non-specific endonuclease domain of a restriction endonuclease (e.g., FokI) fused to a DNA-binding domain from an engineered zinc finger motif. The DNA-binding domain can be engineered to bind a specific sequence in the host's genome and the endonuclease domain makes a double-stranded cut. The donor DNA carries the transgene, for example any of the nucleic acids encoding a CAR or DAR construct described herein, and flanking sequences that are homologous to the regions on either side of the intended insertion site in the host cell's genome. The host cell's DNA repair machinery enables precise insertion of the transgene by homologous DNA repair. Transgenic mammalian host cells have been prepared using zinc finger nucleases (U.S. Pat. Nos. 9,597,357, 9,616,090, 9,816,074 and 8,945,868). A transgenic host cell can be prepared using TALEN (Transcription Activator-Like Effector Nucleases) which are similar to zinc finger nucleases in that they include a non-specific endonuclease domain fused to a DNA-binding domain which can deliver precise transgene insertion. Like zinc finger nucleases, TALEN also introduce a double-strand cut into the host's DNA. Transgenic host cells can be prepared using a meganuclease which acts as a site-specific, rare-cutting endonuclease that recognizes a recognition site on double-stranded DNA about 12-40 base pairs in length. Meganucleases include those from the LAGLIDADG family found most often in mitochondria and chloroplasts of eukaryotic unicellular organisms. An example of a Meganuclease system used to modify genomes is described for example in U.S. Pat. No. 9,889,160. Transgenic host cells can be prepared using CRISPR (Clustered Regularly Interspaced Short Palindromic Repeats). CRISPR employs a Cas endonuclease coupled to a guide RNA for target specific donor DNA integration. The guide RNA includes a conserved multi-nucleotide containing protospacer adjacent motif (PAM) sequence upstream of the gRNA-binding region in the target DNA and hybridizes to the host cell target site where the Cas endonuclease cleaves the double-stranded target DNA. The guide RNA can be designed to hybridize to a specific target site. Similar to zinc finger nuclease and TALEN, the CRISPR/Cas system can be used to introduce site specific insertion of donor DNA having flanking sequences that have homology to the insertion site. Examples of CRISPR/Cas systems used to modify genomes are described for example in U.S. Pat. Nos. 8,697,359, 10,000,772, 9,790,490, and U. S. Patent Application Publication No. US 2018/0346927. In one embodiment, transgenic host cells can be prepared using zinc finger nuclease, TALEN or CRISPR/Cas system, and the host target site can be a TRAC gene (T Cell Receptor Alpha Constant). The donor DNA can include for example any of the nucleic acids encoding a CAR or DAR construct described herein. Electroporation, nucleofection or lipofection can be used to co-deliver into the host cell the donor DNA with the zinc finger nuclease, TALEN or CRISPR/Cas system.

Transgenic host cells can be prepared by transducing host cells (e.g., T cells) with a retroviral vector carrying a nucleic acid encoding a CAR or DAR construct. The transduction can be performed essentially as described in Ma et al., 2004 The Prostate 61:12-25; and Ma et al., The Prostate 74(3):286-296, 2014 (the disclosures of which are incorporated by reference herein in their entireties). The retroviral vector can be transfected into a Phoenix-Eco cell line (ATCC) using FuGene reagent (Promega, Madison, Wis.) to produce Ecotropic retrovirus, then harvest transient viral supernatant (Ecotropic virus) can be used to transduce PG13 packaging cells with Gal-V envelope to produce retrovirus to infect human cells. Viral supernatant from the PG13 cells can be used to transduce activated T cells (or PBMCs) two to three days after CD3 or CD3/CD28 activation. Activated human T cells can be prepared by activating normal healthy donor peripheral blood mononuclear cells (PBMC) with 100 ng/ml mouse anti-human CD3 antibody OKT3 (Orth Biotech, Rartian, N.J.) or anti-CD3ζ, anti-CD28 TransAct (Miltenyi Biotech, German) as manufacturer's manual and 300-1000 U/ml IL2 in AIM-V growth medium (GIBCO-Thermo Fisher scientific, Waltham, Mass.) supplemented with 5% FBS for two days. Approximately 5×10⁶ activated human T cells can be transduced in a 10 ug/ml retronectin (Takara Bio USA) pre-coated 6-well plate with 3 ml viral supernatant and centrifuged at 1000 g for about 1 hour at approximately 32° C. After transduction, the transduced T cells can be expanded in AIM-V growth medium supplemented with 5% FBS and 300-1000 U/ml IL2.

A host cell can be a prokaryote, for example, E. coli, or it can be a eukaryote, for example, a single-celled eukaryote (e.g., a yeast or other fungus), a plant cell (e.g., a tobacco or tomato plant cell), an mammalian cell (e.g., a human cell, a monkey cell, a hamster cell, a rat cell, a mouse cell, or an insect cell) or a hybridoma. In one embodiment, a host cell can be introduced with an expression vector operably linked to a nucleic acid encoding a desired antibody thereby generating a transfected/transformed host cell which is cultured under conditions suitable for expression of the antibody by the transfected/transformed host cell, and optionally recovering the antibody from the transfected/transformed host cells (e.g., recovery from host cell lysate) or recovery from the culture medium. In one embodiment, host cells comprise non-human cells including CHO, BHK, NS0, SP2/0, and YB2/0. In one embodiment, host cells comprise human cells including HEK293, HT-1080, Huh-7 and PER.C6. Examples of host cells include the COS-7 line of monkey kidney cells (ATCC CRL 1651) (see Gluzman et al., 1981, Cell 23: 175), L cells, C127 cells, 3T3 cells (ATCC CCL 163), Chinese hamster ovary (CHO) cells or their derivatives such as Veggie CHO and related cell lines which grow in serum-free media (see Rasmussen et al., 1998, Cytotechnology 28:31) or CHO strain DX-B 11, which is deficient in DHFR (see Urlaub et al., 1980, Proc. Natl. Acad. Sci. USA 77:4216-20), HeLa cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo 205 cells, other transformed primate cell lines, normal diploid cells, cell strains derived from in vitro culture of primary tissue, primary explants, HL-60, U937, HaK or Jurkat cells. In one embodiment, host cells include lymphoid cells such as Y0, NS0 or Sp20. In one embodiment, a host cell is a mammalian host cell, but is not a human host cell. Typically, a host cell is a cultured cell that can be transformed or transfected with a polypeptide-encoding nucleic acid, which can then be expressed in the host cell. The phrase “transgenic host cell” or “recombinant host cell” can be used to denote a host cell that has been introduced (e.g., transduced, transformed or transfected) with an exogenous nucleic acid either to be expressed or not to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein. A host cell, or a population of host cells, harboring a vector (e.g., an expression vector) operably linked to at least one nucleic acid encoding one or more polypeptides that comprise a dimeric antigen receptor (DAR) or antigen-binding portions thereof are described herein.

The host cell or the population of host cells comprise T lymphocytes (e.g., T cells, regulatory T cells, gamma-delta T cells, and cytotoxic T cells), NK (natural killer) cells, macrophages, dendritic cells, mast cells, eosinophils, B lymphocytes, monocytes. In one embodiment, the NK cells comprise cord blood-derived NK cells, or placental derived NK cells.

Polypeptides of the present disclosure (e.g., dimeric antigen receptors (DAR)) can be produced using any method known in the art. In one example, the polypeptides are produced by recombinant nucleic acid methods by inserting a nucleic acid sequence (e.g., DNA) encoding the polypeptide into a recombinant expression vector which is introduced into a host cell and expressed by the host cell under conditions promoting expression.

General techniques for recombinant nucleic acid manipulations are described for example in Sambrook et al., in Molecular Cloning: A Laboratory Manual, Vols. 1-3, Cold Spring Harbor Laboratory Press, 2 ed., 1989, or F. Ausubel et al., in Current Protocols in Molecular Biology (Green Publishing and Wiley-Interscience: New York, 1987) and periodic updates, herein incorporated by reference in their entireties. The nucleic acid (e.g., DNA) encoding the polypeptide is operably linked to an expression vector carrying one or more suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences that control the termination of transcription and translation. The expression vector can include an origin or replication that confers replication capabilities in the host cell. The expression vector can include a gene that confers selection to facilitate recognition of transgenic host cells (e.g., transformants).

The recombinant DNA can also encode any type of protein tag sequence that may be useful for purifying the protein. Examples of protein tags include but are not limited to a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, N.Y., 1985).

The expression vector construct can be introduced into the host cell using a method appropriate for the host cell. A variety of methods for introducing nucleic acids into host cells are known in the art, including, but not limited to, electroporation; transfection employing calcium chloride, rubidium chloride, calcium phosphate, DEAE-dextran, or other substances; viral transfection; non-viral transfection; microprojectile bombardment; lipofection; and infection (e.g., where the vector is an infectious agent). Suitable host cells include prokaryotes, yeast, mammalian cells, or bacterial cells.

Suitable bacteria include gram negative or gram positive organisms, for example, E. coli or Bacillus spp. Yeast, for example from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow and Summers, (Bio/Technology, 6:47, 1988). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. The protein is then purified from culture media or cell extracts. Any of the polypeptide chains that comprise the dimeric antigen receptors (DAR) or antigen-binding portions thereof, can be expressed by transgenic host cells.

Antibodies and antigen binding proteins disclosed herein can also be produced using cell-translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system.

Nucleic acids encoding any of the various polypeptides disclosed herein may be synthesized chemically. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc. Natl. Acad. Sci. USA. 2003 100(2):438-42; Sinclair et al. Protein Expr. Purif. 2002 (1):96-105; Connell N D. Curr. Opin. Biotechnol. 2001 12(5):446-9; Makrides et al. Microbiol. Rev. 1996 60(3):512-38; and Sharp et al. Yeast. 1991 7(7):657-78.

Antibodies and antigen binding proteins described herein can also be produced by chemical synthesis (e.g., by the methods described in Solid Phase Peptide Synthesis, 2nd ed., 1984, The Pierce Chemical Co., Rockford, Ill.). Modifications to the protein can also be produced by chemical synthesis.

Antibodies and antigen binding proteins described herein can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis.

In one embodiment, a preparation of transgenic DAR T cells can be enriched for T cells that express a dimeric antigen receptor (DAR) construct. For example, anti-BCMA DAR T cells can be prepared from PBMCs to generate a T cell population containing a mixture of non-transgenic T cells and transgenic T cells. The transgenic T cells expressing anti-BCMA DAR constructs can be enriched to reduce the percent or number of non-transgenic T cells using cell sorting (e.g., fluorescence-activated cell sorting), gradient purification, or culture methods suitable for preferentially inducing proliferation of transgenic T cells over non-transgenic T cells. In one embodiment, the enrichment step increases the number of transgenic DAR T cells compared to non-transgenic T cells by about 2-5 fold, or about 5-10 fold, or about 10-15 fold, or about 15-20 fold, or about 20-50 fold, or higher-fold levels of enrichment.

In certain embodiments, the antibodies and antigen binding proteins described herein (e.g., DAR) can further comprise post-translational modifications. Exemplary post-translational protein modifications include phosphorylation, acetylation, methylation, ADP-ribosylation, ubiquitination, glycosylation, afucosylation, carbonylation, sumoylation, biotinylation or addition of a polypeptide side chain or of a hydrophobic group. As a result, the modified polypeptides may contain non-amino acid elements, such as lipids, poly- or mono-saccharide, and phosphates. In one embodiment, glycosylation can be sialylation, which conjugates one or more sialic acid moieties to the polypeptide. Sialic acid moieties improve solubility and serum half-life while also reducing the possible immunogenicity of the protein. See Raju et al. Biochemistry. 2001 31; 40(30):8868-76.

The present disclosure provides therapeutic compositions comprising any of the dimeric antigen receptors (DAR) or antigen-binding portions thereof or transgenic host cells (e.g., expressing a DAR) that are described herein in an admixture with a pharmaceutically-acceptable excipient. An excipient encompasses carriers, stabilizers and excipients. Excipients of pharmaceutically acceptable excipients includes for example inert diluents or fillers (e.g., sucrose and sorbitol), lubricating agents, glidants, and anti-adhesives (e.g., magnesium stearate, zinc stearate, stearic acid, silicas, hydrogenated vegetable oils, or talc). Additional examples include buffering agents, stabilizing agents, preservatives, non-ionic detergents, anti-oxidants and isotonifiers. Where a therapeutic composition comprises cells, the pharmaceutically-acceptable excipients will be chosen so as not to interfere with the viability or activity of the cells.

Therapeutic compositions and methods for preparing them are well known in the art and are found, for example, in “Remington: The Science and Practice of Pharmacy” (20th ed., ed. A. R. Gennaro A R., 2000, Lippincott Williams & Wilkins, Philadelphia, Pa.). Therapeutic compositions can be formulated for parenteral administration may, and can for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the antibody (or antigen binding protein thereof) described herein. Nanoparticulate formulations (e.g., biodegradable nanoparticles, solid lipid nanoparticles, liposomes) may be used to control the biodistribution of the antibody (or antigen binding protein thereof). Other potentially useful parenteral delivery systems include ethylene-vinyl acetate copolymer particles, osmotic pumps, implantable infusion systems, and liposomes. The concentration of the antibody (or antigen binding protein thereof) in the formulation varies depending upon a number of factors, including the dosage of the drug to be administered, and the route of administration.

Any of the dimeric antigen receptors (DAR) or antigen-binding portions thereof described herein may be administered as a pharmaceutically acceptable salt, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like. In one example, the DAR (or antigen binding portions thereof) is formulated in the presence of sodium acetate to increase thermal stability.

The term “subject” as used herein refers to human and non-human animals, including vertebrates, mammals and non-mammals. In one embodiment, the subject can be human, non-human primates, simian, ape, murine (e.g., mice and rats), bovine, porcine, equine, canine, feline, caprine, lupine, ranine or piscine.

The term “administering”, “administered” and grammatical variants refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In one embodiment, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods. Any of the dimeric antigen receptors (DAR) or antigen-binding portions thereof described herein can be administered to a subject using art-known methods and delivery routes.

The terms “effective amount”, “therapeutically effective amount” or “effective dose” or related terms may be used interchangeably and refer to an amount of any of the dimeric antigen receptors (DAR) described herein that when administered to a subject, is sufficient to effect a measurable improvement or prevention of a disease or disorder associated with tumor or cancer antigen expression. Therapeutically effective amounts of DAR provided herein, when used alone or in combination, will vary depending upon the relative activity of the antibodies and combinations (e.g., in inhibiting cell growth) and depending upon the subject and disease condition being treated, the weight and age and sex of the subject, the severity of the disease condition in the subject, the manner of administration and the like, which can readily be determined by one of ordinary skill in the art.

In one embodiment, a therapeutically effective amount will depend on certain aspects of the subject to be treated and the disorder to be treated and may be ascertained by one skilled in the art using known techniques. In general, the DAR T cells can be administered to the subject at about 10³-10⁴ cells/kg, or about 10⁴-10⁵ cells/kg, or about 10⁵-10⁶ cells/kg, or about 10⁶-10⁷ cells/kg, or about 10⁷-10⁸ cells/kg, or about 10⁸-10⁹ cells/kg, or about 10⁹-10¹² cells/kg. The DAR T cells can be administered only once, or daily (e.g., once, twice, three times, or four times daily), or less frequently (e.g., weekly, every two weeks, every three weeks, monthly, or quarterly). In addition, as is known in the art, adjustments for age as well as the body weight, general health, sex, diet, time of administration, drug interaction, and the severity of the disease may be necessary.

In one embodiment, a therapeutically effective amount comprises a dose of about 10³-10¹² transgenic host cells administered to the subject. In one embodiment, the transgenic host cells harbor one or more expression vectors that express the polypeptide chains that comprise any of the DARs described herein. The therapeutically effective amount can be determined by considering the subject to receive the therapeutically effective amount and the disease/disorder to be treated which may be ascertained by one skilled in the art using known techniques. The therapeutically effective amount may consider factors pertaining to the subject such as age, body weight, general health, sex, diet, time of administration, drug interaction, and the severity of the disease/disorder. The therapeutically effective amount may consider the purity of the transgenic host cells, which can be about 65%-98% or higher levels of purity. The therapeutically effective amount of the transgenic host cells can be administered to the subject at least once, or twice, three times, 4 times, 5 times, or more over a period of time. The period of time can be per day, per week, per month, or per year. The therapeutically effective amount of the transgenic cells administered to the subject can be same each time or can be increased or decreased at each administration event. The therapeutically effective amount of the transgenic cells can be administered to the subject until the tumor size or number of cancer cells is reduced by 5%-90% or more, compared to the tumor size or number of cancer cells prior to administration of the transgenic host cells.

The present disclosure provides methods for treating a subject having a disease/disorder associated with expression or over-expression of one or more tumor-associated antigens. The disease comprises cancer or tumor cells expressing the tumor-associated antigens, such as for example BCMA antigen. In one embodiment, the cancer or tumor includes cancer of the prostate, breast, ovary, head and neck, bladder, skin, colorectal, anus, rectum, pancreas, lung (including non-small cell lung and small cell lung cancers), leiomyoma, brain, glioma, glioblastoma, esophagus, liver, kidney, stomach, colon, cervix, uterus, endometrium, vulva, larynx, vagina, bone, nasal cavity, paranasal sinus, nasopharynx, oral cavity, oropharynx, larynx, hypolarynx, salivary glands, ureter, urethra, penis and testis.

In one embodiment, the cancer comprises hematological cancers, including leukemias, lymphomas, myelomas and B cell lymphomas. Hematologic cancers include multiple myeloma (MM), non-Hodgkin's lymphoma (NHL) including Burkitt's lymphoma (BL), B chronic lymphocytic leukemia (B-CLL), systemic lupus erythematosus (SLE), B and T acute lymphocytic leukemia (ALL), acute myeloid leukemia (AML), chronic lymphocytic leukemia (CLL), diffuse large B cell lymphoma, chronic myelogenous leukemia (CML), hairy cell leukemia (HCL), follicular lymphoma, Waldenstrom's Macroglobulinemia, mantle cell lymphoma, Hodgkin's Lymphoma (HL), plasma cell myeloma, precursor B cell lymphoblastic leukemia/lymphoma, plasmacytoma, giant cell myeloma, plasma cell myeloma, heavy-chain myeloma, light chain or Bence-Jones myeloma, lymphomatoid granulomatosis, post-transplant lymphoproliferative disorder, an immunoregulatory disorder, rheumatoid arthritis, myasthenia gravis, idiopathic thrombocytopenia purpura, anti-phospholipid syndrome, Chagas' disease, Grave's disease, Wegener's granulomatosis, poly-arteritis nodosa, Sjogren's syndrome, pemphigus vulgaris, scleroderma, multiple sclerosis, anti-phospholipid syndrome, ANCA associated vasculitis. Goodpasture's disease, Kawasaki disease, autoimmune hemolytic anemia, and rapidly progressive glomerulonephritis, heavy-chain disease, primary or immunocyte-associated amyloidosis, and monoclonal gammopathy of undetermined significance.

### Dimeric Antigen Receptors (DARs)

The present disclosure provides dimeric antigen receptors (DARs) comprising a Fab fragment joined to a transmembrane region and intracellular regions. In one embodiment, the DAR construct includes an optional hinge region between the Fab fragment and the transmembrane region. In some embodiments, the presently disclosed DAR structures provide unexpected and surprising results, e.g., based on comparing a DAR structure having a Fab format antibody to a CAR structure having an scFv format of the same antibody. Moreover, the DAR and CAR formats can be directly compared because the hinge regions, transmembrane regions and two intracellular regions can be the same. Yet the DAR format can provide superior results relative to the corresponding CAR format in binding (e.g., specific binding) to cells expressing the target antigen, antigen-induced cytokine release and/or antigen-induced cytotoxicity.

The present disclosure provides dimeric antigen receptor (DAR) constructs comprising a heavy chain binding region on one polypeptide chain and a light chain binding region on a separate polypeptide chain. The two polypeptide chains that make up the dimeric antigen receptors can dimerize to form a protein complex that more closely mimics a Fab structure compared to an scFv. The dimeric antigen receptors have antibody-like properties as they bind specifically to a target antigen. In the dimeric antigen receptor (DAR) constructs, the heavy chain variable and constant regions on one polypeptide chain can lack an intervening linker sequence, and the light chain variable and constant regions on the separate polypeptide chain can also lack an intervening linker sequence. The lack of intervening linker sequences on the DAR polypeptide chains may reduce immunogenicity compared to an scFv which contains an intervening linker sequence between the variable heavy and variable light chain regions. The dimeric antigen receptors have antibody-like properties as they bind specifically to a target antigen. The dimeric antigen receptors can be used for directed cell therapy.

The present disclosure provides transgenic T cells engineered to express anti-BCMA dimeric antigen receptor (DAR) constructs having an antigen-binding extracellular portion, optional hinge portion, transmembrane portion, and an intracellular portion having co-stimulatory and/or intracellular signaling regions. The extracellular portion exhibits high affinity and avidity to bind BCMA-expressing diseased hematopoietic cells leading to T cell activation and diseased-cell killing, while sparing normal cells. The intracellular portion of the anti-BCMA DAR constructs comprises co-stimulatory and/or signaling regions that mediate T cell activation upon antigen binding which can lead to formation of memory T cells, enhanced T cell expansion (e.g., memory T cell expansion), and/or reduced T cells exhaustion. It is postulated that formation of memory T cells is important to prevent disease relapse in a subject suffering from a disease involving BCMA-overexpression. Described herein are multiple configurations of DAR constructs that differ in the type and number of intracellular co-stimulatory and signaling regions, providing flexibility in designing DAR constructs for producing a strong and rapid effector response (e.g., DAR constructs comprising an intracellular CD28 co-stimulatory region) and/or generating a longer-lasting memory T cell population (e.g., DAR constructs comprising an intracellular 4-1BB co-stimulatory region).

In one embodiment, the population of transgenic T cells expressing anti-BCMA DAR comprise a mixture of CD4+ and CD8+ T cells which are either naïve T cells (T_{N}) or antigen-experienced T cells at various stages of differentiation into memory T cells (T_{M}). The population of memory T cells (T_{M}) is heterogenous containing subset populations of central memory (T_{CM}) and effector memory (T_{EM}) T cells which differ in their cell receptor expression patterns, and may exhibit varying degrees of anti-tumor potency, in vitro proliferative capacity and in vivo persistence. Generally, cell receptor expression patterns on human naïve T cells (T_{N}) include CD62L+, CCR7+, CD45RA+, CD45RO− and CD27+. Central memory T cells (T_{CM}) are CD62L+, CCR7+, CD45RA+, CD45RO+ and CD27+. Effector memory T cells (T_{EM}) are CD62L−, CCR7−, CD45RA−, CD45RO− and CD27−. By contrast, effector T cells (T_{E}) are CD62L−, CCR7−, CD45RO− and CD27−. It is postulated that antigen-experienced memory T cells differentiate from a stem cell-like memory T cell (T_{SCM}) to central memory T cells (T_{CM}) to effector memory T cells (T_{EM}) to terminally differentiated effector T cells. Central memory T cells (T_{CM}) are classified as early differentiated progenitors, can self renew (regenerate), and can maintain long-lived stem cell-like T cell memory properties. Effector memory T cells (T_{EM}) appear to be more differentiated than central memory T cells (T_{CM}) which can differentiate into terminally differentiated effector T cells (T_{E}) that are cytotoxic, generate inflammatory cytokines and have little proliferative capacity. In response to antigen stimulation, CD8+ central memory (T_{CM}) and effector memory (T_{EM}) T cells can differentiate into cytolytic effector T cells (T_{E}) that express elevated levels of perforin and granzymes (e.g., granzymes A and/or B) and are short-lived. Thus, it is advantageous to generate a population of anti-BCMA DAR T cells containing an increased level of central memory (T_{CM}) and effector memory (T_{EM}) T cells which exhibit anti-tumor potency, increased in vitro proliferative capacity and in vivo persistence, compared to cytolytic effector T cells (T_{E}).

In one embodiment, the transgenic DAR T cells described herein comprise CD8+ and CD4+ memory T cells that exhibit characteristic cell receptor expression patterns of central memory T cells (T_{CM}) and effector memory T cells (T_{EM}) and have the properties that make them suitable for in vivo adoptive transfer as they exhibit anti-tumor potency, in vitro proliferative capacity and in vivo persistence.

In one embodiment, the transgenic anti-BCMA DAR T cells can be administered to a subject having a tumor or cancer over-expressing BCMA antigen in order to reduce tumor burden. In one embodiment, the transgenic DAR T cells can be administered to the subject in a single dose or multiple doses. The anti-BCMA DAR T cells can expand in the subject (e.g., in vivo) which may, or may not, correlate with the presence of DAR T cells having memory T cell properties. In one embodiment, anti-BCMA DAR T cells can expand in vivo in the treated subject after a single dose. The expansion can be detected days, weeks, or months post-treatment. The anti-BCMA DAR T cells can persist in the subject days, weeks or months post-treatment.

In one embodiment, functional persistence of the transgenic anti-BCMA DAR T cells in a subject confers long-term tumor immunity for days, weeks, or months. The level of persistence can be assessed by conducting a tumor re-challenge experiment in an animal model. For example, a single dose of anti-BCMA DAR T cells can be administered to at least one animal subject having primary tumor burden. After the primary tumor burden is reduced, the animals are re-challenged with secondary tumor cells, and secondary tumor burden is monitored. A delay in secondary tumor growth (tumor relapse) or tumor elimination indicates that a single dose of the anti-BCMA DAR T cells are persistent in vivo, and may indicate long-term in vivo expansion. The delay may be measured in days, weeks or months. Human subjects that receive anti-BCMA DAR T cells for tumor treatment may also benefit from long-term tumor immunity for days, weeks or months.

In one embodiment, the population of transgenic T cells expressing anti-BCMA DAR exhibit reduced levels of T cell exhaustion compared to transgenic T cells expressing an anti-BCMA CAR (chimeric antigen receptor). In one embodiment, a reduced percentage of the DAR T cells in a population of DAR T cells exhibit T cell exhaustion compared to a population of CAR T cells. T cell exhaustion refers to a state of dysfunction caused by persistent antigen stimulation. In both CD8+ and CD4+ DAR T cells, exhaustion is characterized by co-expression of inhibitory receptors, including any combination of two or more of PD-1, CTLA4, LAGS, TIM3, 2B4/CD244/CD244/SLAMF4, CD160 and/or TIGIT. T cell exhaustion in DAR T cells is also characterized by loss of IL-2 production, severely reduced or loss of proliferative capacity and cytolytic activity. In CD8+ T cells, exhaustion can lead to T cell death. T cell exhaustion is postulated to represent late-stage T cell differentiation. T cell exhaustion is believed to be a cause of CAR T cell therapy failure.

In one embodiment, the number of DAR T cells exhibiting T cell exhaustion receptor markers (e.g., any combination of two or more of PD-1, CTLA4, LAGS, TIM3, 2B4/CD244/CD244/SLAMF4, CD160 and/or TIGIT) is reduced compared to the number of CAR T cells exhibiting the same T cell exhaustion receptors, where the reduction is about 2-fold, or about 3-fold, or about 4-fold, or about 5-fold, or higher-fold reduction levels, or less than about 2-fold reduction levels.

In one embodiment, the anti-BCMA DAR T cells can be prepared from a polyclonal T cell population (e.g., PBMCs) without pre-enrichment of naïve or memory T cell populations (e.g., central memory or effector memory T cells). Pre-enrichment procedures can include cell culture methods, cell sorting (e.g., fluorescence-activated cell sorting), or gradient purification.

The transgenic anti-BCMA DAR T cells can be prepared and then stored for future use in an in vitro assay or for administration to a subject. In one embodiment, the anti-BCMA DAR T cells can be stored under cryopreservation conditions for hours, days or months. In one embodiment, the cryopreserved anti-BCMA DAR T cells can be thawed, and the thawed DAR T cells retain similar levels of viability and function compared to freshly-prepared anti-BCMA DAR T cells that are not cryopreserved and thawed. In one embodiment, the anti-BCMA DAR T cells are cryopreserved for about 1-24 hours. In one embodiment, the anti-BCMA DAR T cells are cryopreserved for about 1-30 days. In one embodiment, the anti-BCMA DAR T cells are cryopreserved for about 1-2 months, or about 2-3 months, or about 3-4 months, or about 4-5 months, or about 5-6 months, or more than 6 months. In one embodiment, the anti-BCMA DAR T cells can be cryopreserved at temperature ranges of about −80 to −100° C., or about −100 to −150° C. In one embodiment, cryopreserved anti-BCMA DAR T cells can be thawed and retain viability, where about 55-65% of the thawed cells are viable, or about 65-75%, or about 75-85% or about 85-95%, or about 95-99% of the thawed cells are viable.

In one embodiment, anti-BCMA DAR T cells can be cryopreserved in freezing medium comprising 70% AIM-V medium, 20% FBS and 10% DMSO. In one embodiment, about 1×10⁵-1×10⁹ anti-BCMA DAR T cells can be cryopreserved in freezing medium. In one embodiment, the anti-BCMA DAR T cells can be resuspended in freezing medium and placed at −80° C. overnight, and then transferred to −150° C. for storage. In one embodiment, the anti-BCMA DAR T cells can be resuspended in freezing medium and placed directly at −80° C. or −150° C. for storage. In one embodiment, the cryopreserved anti-BCMA DAR T cells can be place at 37° C. until thawed, and then placed on ice until ready for use.

The present disclosure provides dimeric antigen receptors (DAR) constructs having first and second polypeptide chains that associate with each other to form an antigen binding domain that binds a BCMA protein (e.g., target antigen). In one embodiment, the BCMA protein is from human, ape (e.g., chimpanzee), monkey (e.g., cynomolgus), murine (e.g., mouse and/or rat), canine (e.g., dog) and/or feline (e.g., cat). In one embodiment, the BCMA protein comprises human BCMA (e.g., UniProt Q02223). In one embodiment, the BCMA protein comprises wild type human (e.g., SEQ ID NO:1) or a mutant human BCMA protein (e.g., SEQ ID NO:2 or 3). In one embodiment, the dimeric antigen receptor (DAR) binds the wild type human BCMA protein (SEQ ID NO:1) or any portion thereof, but does not bind mutant BCMA proteins (SEQ ID NOS:2 and 3). In one embodiment, the dimeric antigen receptors (DAR) constructs can bind APRIL (A PRoliferation-Inducing Ligand) (e.g., UniProtKB 075888 TNF13 Human, SEQ ID NO:4) and/or BAFF (e.g., UniProt Q9Y275 TN13B human, SEQ ID NO:5).

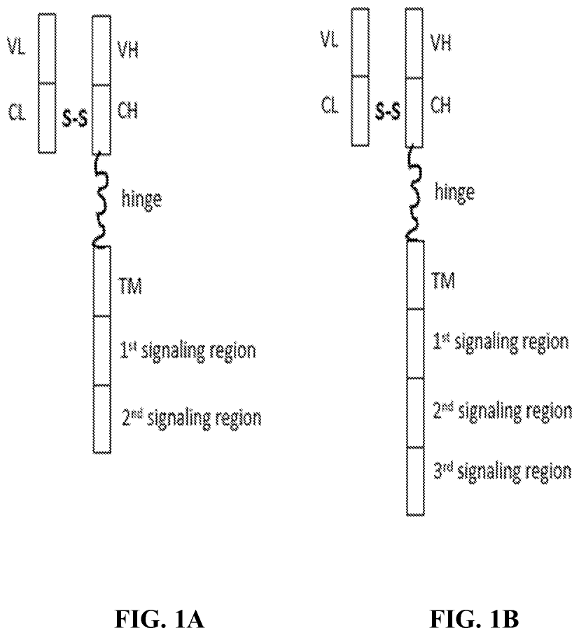

The present disclosure provides a structure for a DAR (dimeric antigen receptor) construct having a first polypeptide chain and a second polypeptide chain, wherein the first polypeptide chain comprises a heavy chain variable region of an antibody and the second polypeptide chain comprises a light chain variable region of an antibody, wherein the first polypeptide chain is linked to the second polypeptide chain by one or a plurality of disulfide bonds at regions outside of a transduced cell when both the first polypeptide chain and the second polypeptide chain are expressed by a same cell. In some embodiments, a DAR construct comprises a first polypeptide chain comprising, in sequence, an antibody heavy chain with a variable domain region and a CH1 region, a hinge region, a transmembrane region, and an intracellular region having 2-5 signaling domains, and a second polypeptide chain comprising, and an antibody light chain variable domain region (kappa (K) or lambda (L)) with a corresponding CL/CK region, wherein the CH1 and CL/CK regions in each first and second polypeptide chains are linked with one or two disulfide bonds (e.g., see FIGS. 1A and B).

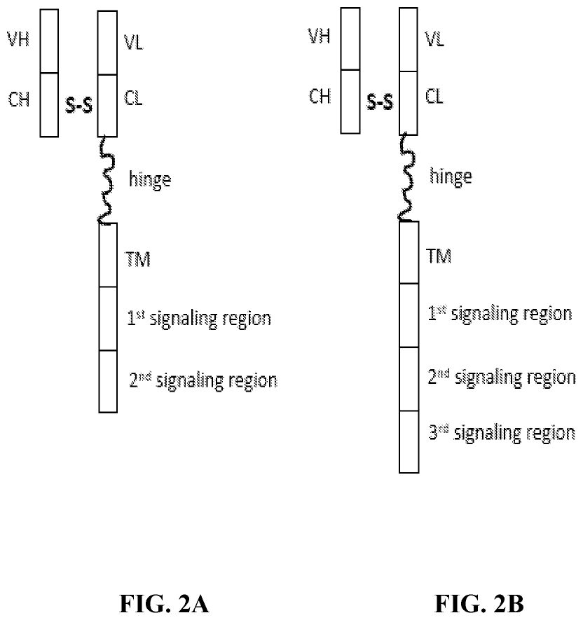

The present disclosure provides a structure for a DAR (dimeric antigen receptor) construct having a first polypeptide chain and a second polypeptide chain, wherein the first polypeptide chain comprises a light chain variable region of an antibody and the second polypeptide chain comprises a heavy chain variable region of an antibody, wherein the first polypeptide chain is linked to the second polypeptide chain by one or a plurality of disulfide bonds at regions outside of a transduced cell when both the first polypeptide chain and the second polypeptide chain are expressed by a same cell. In some embodiments, a DAR construct comprises a first polypeptide chain comprising, in sequence, an antibody light chain with a variable domain region (kappa (K) or lambda (L)) with a corresponding CL/CK region, a hinge region, a transmembrane region, and an intracellular region having 2-5 signaling domains, and a second polypeptide chain comprising, and an antibody heavy chain variable domain region and a CH1 region, wherein the CL/CK and CH1 regions in each first and second polypeptide chains are linked with one or two disulfide bonds (e.g., see FIGS. 2A and B).

In one embodiment, the DAR construct comprises an antibody heavy chain variable region and an antibody light chain variable region on separate polypeptide chains, wherein the heavy chain variable region and the light chain variable region form an antigen binding domain.

In one embodiment, the hinge region is about 10 to about 100 amino acids in length. In one embodiment, the hinge region is independently selected from the group consisting of a CD8 hinge region or a fragment thereof, a CD8α hinge region or a fragment thereof, a hinge region of an antibody (IgG, IgA, IgM, IgE, or IgD) joining the constant domains CH1 and CH2 of an antibody. The hinge region can be derived from an antibody and may or may not comprise one or more constant regions of the antibody.

In one embodiment, the transmembrane domain can be derived from a membrane protein sequence region selected from the group consisting of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD40L/CD154, VEGFR2, FAS, and FGFR2B.

In one embodiment, the signaling region is selected from the group consisting of signaling regions from CD3-zeta chain, 4-1BB, CD28, CD27, OX40, CD30, CD40, PD-1, ICOS, lymph oocyte function-associated antigen-1 (LFA-1), CD2, CD7, LIGHT, NKG2C, B7-H3, GITR (TNFRSF18), DR3 (TNFRSF25), TNFR2, CD226, and combinations thereof.

In one embodiment, a general design of a dimeric antigen receptor includes a first polypeptide chain and a second polypeptide chain, wherein the first polypeptide chain comprises an antigen binding region connected to a dimerization region, connected to a hinge region, connected to a transmembrane region, and connected to one or a plurality of intracellular sequence region(s), and wherein the second polypeptide chain comprises an antigen binding domain and a dimerization domain. In one embodiment, the antigen binding domain on one or both of the first and the second polypeptide chains is selected from the group consisting of a heavy chain variable region, a light chain variable region, an extracellular region of a cytokine receptor, a single domain antibody, and combinations thereof. In one embodiment, the dimerization domain on one or both of the first and second polypeptide chains is selected from the group consisting of a kappa light chain constant region, a lambda light chain constant region, a leucine zipper, myc-max components, and combinations thereof. In FIGS. 1A-B and 2A-B, the “S—S” represents any chemical bond or association that results in dimerization of the first and second polypeptide chains, including disulfide bond, leucine zipper or myc-max components.

The present disclosure provides dimeric antigen receptors (DAR) constructs where the first polypeptide chain carries the heavy chain variable (VH) and heavy chain constant regions (CH), and the second polypeptide chain carries the light chain variable (VL) and light chain constant regions (CL) (e.g., FIGS. 1A and B). In one embodiment, the dimeric antigen receptors (DAR) construct comprises: (a) a first polypeptide chain comprising a plurality of regions ordered from the amino terminus to the carboxyl terminus: (i) an antibody heavy chain variable region (VH), (ii) an antibody heavy chain constant region (CH), (iii) an optional hinge region, (iv) a transmembrane region (TM), and (v) an intracellular region; (b) a second polypeptide chain comprising a plurality of regions ordered from the amino terminus to the carboxyl terminus: (i) an antibody light chain variable region (VL) (e.g., kappa or lambda), and (ii) an antibody light chain constant region (CL).

The present disclosure provides dimeric antigen receptors (DAR) constructs where the first polypeptide chain carries the light chain variable (VL) and light chain constant regions (CL), and the second polypeptide chain carries the heavy chain variable (VH) and heavy chain constant regions (CH) (e.g., FIGS. 2A and B). In one embodiment, the dimeric antigen receptors (DAR) constructs comprises (a) a first polypeptide chain comprising a plurality of regions ordered from the amino terminus to the carboxyl terminus: (i) an antibody light chain variable region (VL), (ii) an antibody light chain constant region (CL), (iii) an optional hinge region, (iv) a transmembrane region (TM), and (v) an intracellular region; (b) a second polypeptide chain comprising a plurality of regions ordered from the amino terminus to the carboxyl terminus: (i) an antibody heavy chain variable region (VH), and (ii) an antibody heavy chain constant region (CH).

In one embodiment, for the dimeric antigen receptors shown in FIGS. 1A and B, and 2A and B, the antibody heavy chain constant region (CH) and the antibody light chain constant region (CL) can dimerize to form a dimerization domain. In one embodiment, the antibody heavy chain constant region and the antibody light chain constant region dimerize via one or two disulfide bonds.

In one embodiment, for the dimeric antigen receptors shown in FIGS. 1A and B, and in FIGS. 2A and B, the antibody heavy chain variable region (VH) and the antibody light chain variable region (VL) associate with each other to form an antigen binding domain. For example, the antibody heavy chain variable region and the antibody light chain variable region associate with each other when the antibody heavy chain constant region and the antibody light chain constant region dimerize.

In one embodiment, for the dimeric antigen receptors shown in FIGS. 1A and B, and 2A and B, the antigen binding domain, which is formed from the antibody heavy chain variable region and the antibody light chain variable region, binds a target antigen.

In one embodiment, for the dimeric antigen receptors shown in FIGS. 1A and B, and 2A and B, the antibody heavy chain variable region and the antibody light chain variable region are fully human antibody regions, humanized antibody region, or chimeric antibody regions.

In one embodiment, for the dimeric antigen receptors shown in FIGS. 1A and B, and 2A and B, the hinge region is about 10 to about 100 amino acids in length. In one embodiment, the hinge region comprises a hinge region or a fragment thereof from an antibody (e.g., IgG, IgA, IgM, IgE, or IgD). In one embodiment, the hinge region comprises a CD8 (e.g., CD8α) and/or CD28 hinge region or a fragment thereof. In one embodiment, the hinge region comprises a CPPC or SPPC amino acid sequence. In one embodiment, the hinge region comprises both CD8 and CD28 hinge sequences (e.g., long hinge region), only CD8 sequence (short hinge) or only CD28 hinge sequence (e.g., short hinge region). In one embodiment, any of the dimeric antigen receptors shown in FIG. 1A or B, or FIG. 2A or B, lack a hinge region.

In one embodiment, for the dimeric antigen receptors shown in FIGS. 1A and B, and 2A and B, the transmembrane regions of the first and second polypeptide chains can be independently derived from CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD40L/CD154, VEGFR2, FAS, and FGFR2B.

In one embodiment, for the dimeric antigen receptors shown in FIGS. 1A and B, and 2A and B, the intracellular region of the first polypeptide comprises intracellular co-stimulatory and/or signaling sequences in any order and of any combination of 2-5 intracellular sequences from 4-1BB, CD3zeta, CD28, CD27, OX40, CD30, CD40, PD-1, ICOS, lymphocyte function-associated antigen-1 (LFA-1), CD2, CD7, LIGHT, NKG2C, B7-H3, GITR (TNFRSF18), DR3 (TNFRSF25), TNFR2, CD226, and combinations thereof. In one embodiment, the intracellular region comprises any one or any combination of two or more of CD28, 4-1BB and/or CD3-zeta intracellular sequences. In one embodiment, the intracellular region comprises CD28 co-stimulatory and CD3-zeta intracellular signaling sequences, or 4-1BB co-stimulatory and CD3-zeta intracellular signaling sequences. In one embodiment, the CD3-zeta portion of the intracellular signaling region comprises ITAM (immunoreceptor tyrosine-based activation motif) motifs 1, 2 and 3 (e.g., long CD3-zeta). In one embodiment, the CD3-zeta portion of the intracellular signaling region comprises only one of the ITAM motifs such as only ITAM 1, 2 or 3 (e.g., short CD3-zeta).

In one embodiment, the first polypeptide chain of the dimeric antigen receptor (FIGS. 1A and 1B) comprises an antibody heavy chain variable region comprising an amino acid sequence having at least 95% sequence identity to the amino acid sequence of any one of SEQ ID NOS:6, 12, 14, 16, 18, 20, 22, 24, 26, or 28. In one embodiment, the antibody heavy chain constant region comprises sequences derived from a human antibody constant region, e.g., a human CH1 domain. In one embodiment, the antibody heavy chain constant region can be derived from an IgM, IgA, IgG, IgE or IgD antibody. In one embodiment, the antibody heavy chain constant region comprises an amino acid sequence having at least 95% sequence identity to the amino acid sequence of SEQ ID NO:7 or 29. In one embodiment, the antibody heavy chain constant region comprises the amino acid sequence of SEQ ID NO:7 or 29. In one embodiment, the hinge region comprises a CD28 hinge comprising the amino acid sequence of SEQ ID NO:35, or a CD8 hinge comprising the amino acid sequence of SEQ ID NO:34, or a hinge region comprising a CD28 and CD8 hinge sequences of SEQ ID NO:36 (e.g., long hinge). In one embodiment, the first polypeptide lacks a hinge region. In one embodiment, the transmembrane region comprises the amino acid sequence of SEQ ID NO:37 (from CD28), SEQ ID NO:38 (from CD8), SEQ ID NO:39 (from 4-1BB), or SEQ ID NO:40 (from CD3zeta). In one embodiment, the intracellular region comprises the amino acid sequence from any one or any combination of two or more intracellular sequences selected from a group consisting of SEQ ID NO:41 (from 4-1BB), SEQ ID NO:42 (from CD28), SEQ ID NO:43 (from OX40), SEQ ID NO:44 (CD3zeta ITAM 1, 2 and 3), SEQ ID NO:45 (CD3zeta ITAM 1), SEQ ID NO:46 (CD3zeta ITAM 2) and/or SEQ ID NO:47 (CD3zeta ITAM 3). In one embodiment, the first polypeptide chain comprises leader sequence comprising the amino acid sequence of SEQ ID NO:54 or 56, or the first polypeptide lacks a leader sequence.

In one embodiment, the second polypeptide chain of the dimeric antigen receptor (FIGS. 1A and 1B) comprises an antibody light chain variable region comprising an amino acid sequence having at least 95% sequence identity to the amino acid sequence of any one of SEQ ID NO:8, 9, 10, 13, 15, 17, 19, 21, 23, 25, 27 or 30. In one embodiment, the antibody light chain constant region comprises a sequence from a human light chain constant region. In one embodiment, the antibody light chain constant region comprises a sequence from a kappa or lambda light chain constant region. In one embodiment, the antibody light chain constant region comprises an amino acid sequence having at least 95% sequence identity to the amino acid sequence of SEQ ID NO:11 or 31. In one embodiment, the antibody light chain constant region comprises the amino acid sequence of SEQ ID NO:11 or 31. In one embodiment, the second polypeptide chain comprises leader sequence comprising the amino acid sequence of SEQ ID NO:55 or 56, or the second polypeptide lacks a leader sequence.

In one embodiment, the first polypeptide chain of the dimeric antigen receptor (FIGS. 2A and 2B) comprises an antibody light chain variable region comprising the amino acid sequence of any one of SEQ ID NO:8, 9, 10, 13, 15, 17, 19, 21, 23, 25, 27, or 30. In one embodiment, the antibody light chain constant region comprises the amino acid sequence of SEQ ID NO:11 or 31. In one embodiment, the hinge region comprises a CD28 hinge comprising the amino acid sequence of SEQ ID NO:35, or a CD8 hinge comprising the amino acid sequence of SEQ ID NO:34, or a hinge region comprising a CD28 and CD8 hinge sequences of SEQ ID NO:36 (e.g., long hinge). In one embodiment, the first polypeptide lacks a hinge region. In one embodiment, the transmembrane region comprises the amino acid sequence of SEQ ID NO:37 (from CD28), SEQ ID NO:38 (from CD8), SEQ ID NO:39 (from 4-1BB), or SEQ ID NO:40 (from CD3zeta). In one embodiment, the intracellular region comprises the amino acid sequence from any one or any combination of two or more intracellular sequences selected from a group consisting of SEQ ID NO:41 (from 4-1BB), SEQ ID NO:42 (from CD28), SEQ ID NO:43 (from OX40), SEQ ID NO:44 (CD3zeta ITAM 1, 2 and 3), SEQ ID NO:45 (CD3zeta ITAM 1), SEQ ID NO:46 (CD3zeta ITAM 2) and/or SEQ ID NO:47 (CD3zeta ITAM 3). In one embodiment, the first polypeptide chain comprises leader sequence comprising the amino acid sequence of SEQ ID NO:55 or 56, or the first polypeptide lacks a leader sequence.

In one embodiment, the second polypeptide chain of the dimeric antigen receptor (FIGS. 2A and 2B) comprises an antibody heavy chain variable region comprising the amino acid sequence of SEQ ID NO: 6, 12, 14, 16, 18, 20, 22, 24, 26, or 28. In one embodiment, the antibody heavy chain constant region comprises the amino acid sequence of SEQ ID NO:7 or 29. In one embodiment, the second polypeptide chain comprises leader sequence comprising the amino acid sequence of SEQ ID NO:54 or 56, or the second polypeptide lacks a leader sequence.

The present disclosure provides a Version 1 (e.g., V1) dimeric antigen receptors (DAR) construct comprising a first polypeptide chain carrying heavy chain variable (VH) and heavy chain constant regions (CH), and a second polypeptide chain carrying light chain variable (VL) and light chain constant regions (CL) (e.g., FIG. 1), wherein (a) the first polypeptide chain comprising a plurality of regions ordered from the amino terminus to the carboxyl terminus: (i) an antibody heavy chain variable region (VH), (ii) an antibody heavy chain constant region (CH), (iii) a long hinge region comprising CD8 and CD28 hinge sequences (e.g., SEQ ID NO:36), (iv) a transmembrane region (TM) comprising CD28 transmembrane sequence (e.g., SEQ ID NO:37), and (v) an intracellular region comprising CD28 co-stimulatory sequence (e.g., SEQ ID NO:42) and CD3-zeta signaling sequence having ITAM motifs 1, 2 and 3 (e.g., SEQ ID NO:44); (b) a second polypeptide chain comprising a plurality of regions ordered from the amino terminus to the carboxyl terminus: (i) an antibody light chain variable region (VL) (e.g., kappa or lambda), and (ii) an antibody light chain constant region (CL). In one embodiment, the antibody heavy chain variable region (VH) comprises an anti-BCMA heavy chain variable region comprising an amino acid sequence having at least 95% sequence identity to the amino acid sequence of any one of SEQ ID NOS:6, 12, 14, 16, 18, 20, 22, 24, 26, or 28, and the antibody light chain variable region (VL) comprises an anti-BCMA light chain variable region sequence comprising an amino acid sequence having at least 95% sequence identity to the amino acid sequence of any one of SEQ ID NO:8, 9, 10, 13, 15, 17, 19, 21, 23, 25, 27 or 30.

The present disclosure provides a Version 2 (e.g., V2) dimeric antigen receptors (DAR) construct comprising a first polypeptide chain carrying heavy chain variable (VH) and heavy chain constant regions (CH), and a second polypeptide chain carrying light chain variable (VL) and light chain constant regions (CL) (e.g., FIGS. 1 and 2), wherein (a) the first polypeptide chain comprising a plurality of regions ordered from the amino terminus to the carboxyl terminus: (i) an antibody heavy chain variable region (VH), (ii) an antibody heavy chain constant region (CH), (iii) a short hinge region comprising a CD28 hinge sequence (e.g., SEQ ID NO:35), (iv) a transmembrane region (TM) comprising CD28 transmembrane sequence (e.g., SEQ ID NO:37), and (v) an intracellular region comprising either (1) a 4-1BB co-stimulatory sequence (e.g., SEQ ID NO:41) and CD3-zeta having ITAM motifs 1, 2 and 3 (e.g., SEQ ID NO:44), or (2) CD28 (e.g., SEQ ID NO:42) signaling sequence and CD3-zeta having ITAM motifs 1, 2 and 3 (e.g., SEQ ID NO:44), or (3) 4-1BB (e.g., SEQ ID NO:41) signaling sequence and CD28 (e.g., SEQ ID NO:42) signaling sequence and CD3-zeta having ITAM motifs 1, 2 and 3 (e.g., SEQ ID NO:44); (b) a second polypeptide chain comprising a plurality of regions ordered from the amino terminus to the carboxyl terminus: (i) an antibody light chain variable region (VL) (e.g., kappa or lambda), and (ii) an antibody light chain constant region (CL).

In one embodiment, the Version 2a (V2a) DAR construct comprises the intracellular region having the 4-1BB co-stimulatory sequence (e.g., SEQ ID NO:41) and CD3-zeta having ITAM motifs 1, 2 and 3 (e.g., SEQ ID NO:44).

In one embodiment, the Version 2b (V2b) DAR construct comprises the intracellular region having the CD28 (e.g., SEQ ID NO:42) signaling sequence and CD3-zeta having ITAM motifs 1, 2 and 3 (e.g., SEQ ID NO:44).

In one embodiment, the Version 2c (V2c) DAR construct comprises the intracellular region having the 4-1BB (e.g., SEQ ID NO:41) signaling sequence and CD28 (e.g., SEQ ID NO:42) signaling sequence and CD3-zeta having ITAM motifs 1, 2 and 3 (e.g., SEQ ID NO:44).

In one embodiment, the DAR V2a and V2b are second generation DAR constructs, while the DAR V2c is a third generation DAR construct.

In one embodiment, in the DAR V2a, V2b and V2c constructs, the antibody heavy chain variable region (VH) comprises an anti-BCMA heavy chain variable region comprising an amino acid sequence having at least 95% sequence identity to the amino acid sequence of any one of SEQ ID NOS:6, 12, 14, 16, 18, 20, 22, 24, 26, or 28, and the antibody light chain variable region (VL) comprises an anti-BCMA light chain variable region sequence comprising an amino acid sequence having at least 95% sequence identity to the amino acid sequence of any one of SEQ ID NO:8, 9, 10, 13, 15, 17, 19, 21, 23, 25, 27 or 30.

The present disclosure provides a Version 3a, 3b and 3c (e.g., V3a, V3b and V3c) dimeric antigen receptors (DAR) construct comprising a first polypeptide chain carrying heavy chain variable (VH) and heavy chain constant regions (CH), and a second polypeptide chain carrying light chain variable (VL) and light chain constant regions (CL) (e.g., FIG. 1), wherein (a) the first polypeptide chain comprising a plurality of regions ordered from the amino terminus to the carboxyl terminus: (i) an antibody heavy chain variable region (VH), (ii) an antibody heavy chain constant region (CH), (iii) a short hinge region comprising CD28 hinge sequences (e.g., SEQ ID NO:35), (iv) a transmembrane region (TM) comprising CD28 transmembrane sequence (e.g., SEQ ID NO:37), and (v) an intracellular region comprising 4-1BB co-stimulatory sequence (e.g., SEQ ID NO:41) and CD3-zeta signaling sequence having only ITAM motif 3 (e.g., SEQ ID NO:47); (b) a second polypeptide chain comprising a plurality of regions ordered from the amino terminus to the carboxyl terminus: (i) an antibody light chain variable region (VL) (e.g., kappa or lambda), and (ii) an antibody light chain constant region (CL).

In one embodiment, the Version 3a (V3a) DAR construct comprises the intracellular region having the 4-1BB co-stimulatory sequence (e.g., SEQ ID NO:41) and CD3-zeta having ITAM motif 3 (e.g., SEQ ID NO:47).

In one embodiment, the Version 3b (V3b) DAR construct comprises the intracellular region having the CD28 (e.g., SEQ ID NO:42) signaling sequence and CD3-zeta having ITAM motif 3 (e.g., SEQ ID NO:47).

In one embodiment, the Version 3c (V3c) DAR construct comprises the intracellular region having the 4-1BB (e.g., SEQ ID NO:41) signaling sequence and CD28 (e.g., SEQ ID NO:42) signaling sequence and CD3-zeta having ITAM motif 3 (e.g., SEQ ID NO:47).

In one embodiment, the DAR V3a and V3b are second generation DAR constructs, while the DAR V3c is a third generation DAR construct.

In one embodiment, in the DAR V3a, V3b and V3c constructs, the antibody heavy chain variable region (VH) comprises an anti-BCMA heavy chain variable region comprising an amino acid sequence having at least 95% sequence identity to the amino acid sequence of any one of SEQ ID NOS:6, 12, 14, 16, 18, 20, 22, 24, 26, or 28, and the antibody light chain variable region (VL) comprises an anti-BCMA light chain variable region sequence comprising an amino acid sequence having at least 95% sequence identity to the amino acid sequence of any one of SEQ ID NO:8, 9, 10, 13, 15, 17, 19, 21, 23, 25, 27 or 30. In one embodiment, the DAR Version 3b (e.g., V3b) is a third generation DAR construct which includes a CD28 co-stimulatory sequence (e.g., SEQ ID NO:42).

The present disclosure provides a Version 4 (e.g., V4) dimeric antigen receptors (DAR) construct comprising a first polypeptide chain carrying heavy chain variable (VH) and heavy chain constant regions (CH), and a second polypeptide chain carrying light chain variable (VL) and light chain constant regions (CL), wherein (a) the first polypeptide chain comprising a plurality of regions ordered from the amino terminus to the carboxyl terminus: (i) an antibody heavy chain variable region (VH), (ii) an antibody heavy chain constant region (CH), (iii) a transmembrane region (TM) comprising CD28 transmembrane sequence (e.g., SEQ ID NO:37), and (iv) an intracellular region comprising 4-1BB co-stimulatory sequence (e.g., SEQ ID NO:41) and CD3-zeta signaling sequence having only ITAM motif 3 (e.g., SEQ ID NO:47); (b) a second polypeptide chain comprising a plurality of regions ordered from the amino terminus to the carboxyl terminus: (i) an antibody light chain variable region (VL) (e.g., kappa or lambda), and (ii) an antibody light chain constant region (CL). The DAR V4 construct lacks a hinge sequence. In one embodiment, in the DAR V4 construct, the antibody heavy chain variable region (VH) comprises an anti-BCMA heavy chain variable region comprising an amino acid sequence having at least 95% sequence identity to the amino acid sequence of any one of SEQ ID NOS:6, 12, 14, 16, 18, 20, 22, 24, 26, or 28, and the antibody light chain variable region (VL) comprises an anti-BCMA light chain variable region sequence comprising an amino acid sequence having at least 95% sequence identity to the amino acid sequence of any one of SEQ ID NO:8, 9, 10, 13, 15, 17, 19, 21, 23, 25, 27 or 30.

### Precursor Polypeptides

The present disclosure provides precursor polypeptides. In one embodiment, the precursor polypeptide can be processed to become first and second polypeptide chains that associate/assemble to form dimeric antigen receptors (DAR) constructs. In any of the precursor polypeptide embodiments described herein that comprise a self-cleaving sequence, the self-cleaving sequence may be a T2A, P2A, E2A, or F2A sequence. In some embodiments, the self-cleaving sequence is other than a T2A sequence, e.g., the self-cleaving sequence is a P2A, E2A, or F2A sequence.

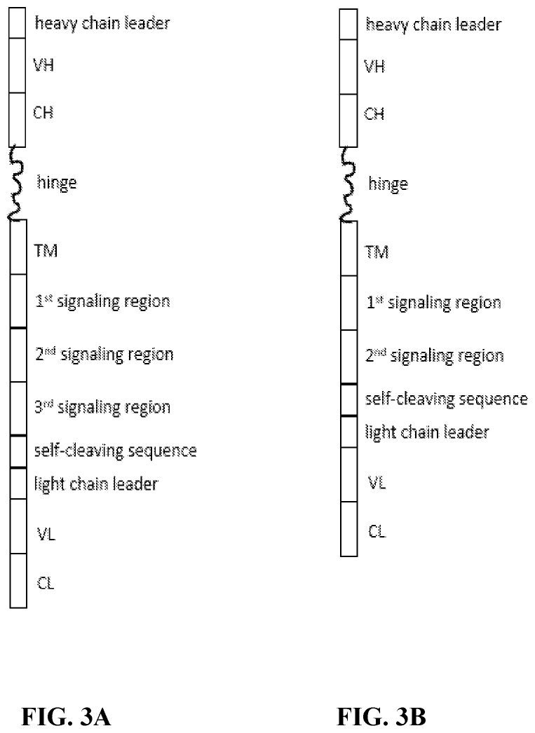

The present disclosure provides precursor polypeptides comprising a plurality of regions ordered from the amino terminus to the carboxyl terminus: (i) a heavy chain leader sequence (ii) an antibody heavy chain variable region, (iii) an antibody heavy chain constant region, (iv) an optional hinge region, (v) a transmembrane region, (vi) an intracellular region, (vii) a self-cleaving sequence, (viii) a light chain leader sequence, (ix) an antibody light chain variable region, and (x) an antibody light chain constant region (FIGS. 3A and B). In a non-limiting example, the intracellular region comprises any combination of at least two of 4-1BB, CD3zeta and/or CD28 (FIGS. 3A and B). The skilled artisan will appreciate that combinations of other intracellular co-stimulatory and/or signaling sequences are possible. The self-cleaving sequence is an amino acid sequence that promotes ribosomal skipping and recommencement of protein translation which generates two separate polypeptides. In one embodiment, a population of precursor polypeptides includes a mixture of polypeptides that have been cleaved at the self-cleaving sequence or not, and/or a mixture of polypeptides that have been cleaved at the heavy chain and/or light chain leader sequences or not.

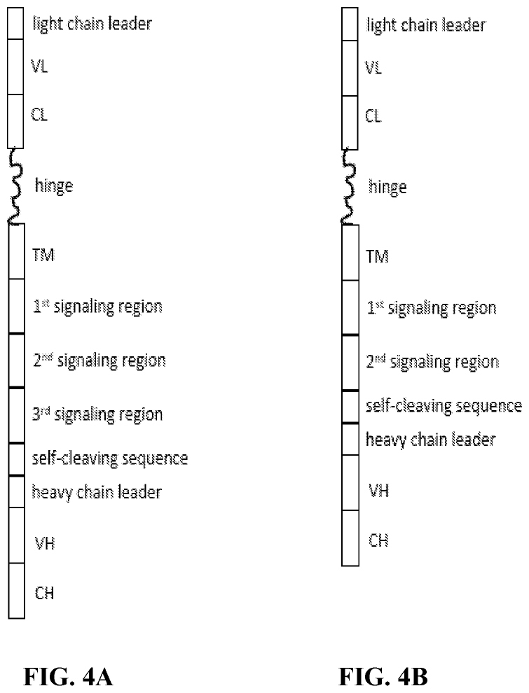

The present disclosure provides precursor polypeptides comprising a plurality of regions ordered from the amino terminus to the carboxyl terminus: (i) a light chain leader sequence (ii) an antibody light chain variable region, (iii) an antibody light chain constant region, (iv) an optional hinge region, (v) a transmembrane region, (vi) an intracellular region, (vii) a self-cleaving sequence, (viii) a heavy chain leader sequence, (ix) an antibody heavy chain variable region, and (x) an antibody heavy chain constant region (FIGS. 4A and B). In a non-limiting example, the intracellular region comprises any combination of at least two of 4-1BB, CD3zeta and/or CD28 (FIGS. 4A and B). The skilled artisan will appreciate that combinations of other intracellular com-stimulatory and/or signaling sequences are possible. The self-cleaving sequence is an amino acid sequence that promotes ribosomal skipping and recommencement of protein translation which generates two separate polypeptides. In one embodiment, a population of precursor polypeptides includes a mixture of polypeptides that have been cleaved at the self-cleaving sequence or not, and/or a mixture of polypeptides that have been cleaved at the heavy chain and/or light chain leader sequences or not.

In one embodiment, for the precursor polypeptides shown in FIGS. 3A and B, and FIGS. 4A and B, the heavy chain and light chain leader sequences comprise peptide signal sequences that target a polypeptide chain (e.g., first and second polypeptide chains) to the secretory pathway of a cell and will allow for integration and anchoring of the polypeptide into the lipid bilayer of the cellular membrane. The heavy and light chain leader sequence can direct transport of the precursor polypeptide from the cytosol to the endoplasmic reticulum of a host cell. The heavy and light chain leader sequence can direct transport of the precursor polypeptide from endoplasmic reticulum to the lipid bilayer of the cellular membrane. The heavy chain and light chain leader sequences include signal sequences comprising CD8α, CD28 or CD16 leader sequences.

In one embodiment, for the precursor polypeptides shown in FIGS. 3A and B, and FIGS. 4A and B, the N-terminal end of a precursor polypeptide includes a first peptide signal sequence (e.g., heavy chain or light chain leader sequence).

In one embodiment, for the precursor polypeptides shown in FIGS. 3A and B, and FIGS. 4A and B, the precursor polypeptide can include a second peptide signal sequence (e.g., heavy chain or light chain leader sequence) located after a cleavage sequence.

In one embodiment, the precursor polypeptide can be cleaved at the cleavage sequence thereby generating first and second polypeptide chains each having a peptide signal sequence at their N-terminal ends.

In one embodiment, for the precursor polypeptides shown in FIGS. 3A and B, and FIGS. 4A and B, the processing of the precursor polypeptide includes cleaving the precursor into first and second polypeptide chains, secreting the precursor, and/or anchoring the precursor in a cellular membrane.

In one embodiment, for the precursor polypeptides shown in FIGS. 3A and B, and FIGS. 4A and B, after the precursor polypeptide chain is cleaved to generate first and second polypeptide chains, the antibody heavy chain constant region (CH) (of one of the polypeptide chains) and the antibody light chain constant region (CL) (of the other polypeptide chain) can dimerize to form a dimerization domain). In one embodiment, the antibody heavy chain constant region and the antibody light chain constant region dimerize via one or two disulfide bonds (e.g., see FIGS. 1A and B, and 2A and B.

In one embodiment, for the precursor polypeptides shown in FIGS. 3A and B, and FIGS. 4A and B, after the precursor polypeptide chain is cleaved to generate first and second polypeptide chains, the antibody heavy chain variable region (VH) (of one of the polypeptide chains) and the antibody light chain variable region (VL) (of the other polypeptide chain) associate with each other to form an antigen binding domain. For example, the antibody heavy chain variable region and the antibody light chain variable region associate with each other when the antibody heavy chain constant region and the antibody light chain constant region dimerize (e.g., see FIGS. 1A and B, and 2A and B).

In one embodiment, for the precursor polypeptides shown in FIGS. 3A and B, and FIGS. 4A and B, the antigen binding domain, which is formed from the antibody heavy chain variable region and the antibody light chain variable region, binds a target antigen.

In one embodiment, for the precursor polypeptides shown in FIGS. 3A and B, and FIGS. 4A and B, the antibody heavy chain variable region and the antibody light chain variable region comprise antibody regions that are fully human, humanized or chimeric.

In one embodiment, for the precursor polypeptides shown in FIGS. 3A and B, and FIGS. 4A and B, the hinge region is about 10 to about 100 amino acids in length. In one embodiment, the hinge region comprises a hinge region or a fragment thereof from an antibody (e.g., IgG, IgA, IgM, IgE, or IgD). In one embodiment, the hinge region comprises a CD8 (e.g., CD8α) or CD28 hinge region or a fragment thereof. In one embodiment, the hinge region comprises a CPPC or SPPC amino acid sequence. In one embodiment, the hinge region comprises both CD8 and CD28 hinge sequences (e.g., long hinge region), only CD8 sequence (short hinge) or only CD28 hinge sequence (e.g., short hinge region). In one embodiment, the precursor polypeptide lacks a hinge region.

In one embodiment, for the precursor polypeptides shown in FIGS. 3A and B, and FIGS. 4A and B, the transmembrane regions of the precursor polypeptide chain can be derived from CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD40L/CD154, VEGFR2, FAS, and FGFR2B.

In one embodiment, for the precursor polypeptides shown in FIGS. 3A and B, and FIGS. 4A and B, the intracellular region of the first polypeptide comprises intracellular signaling and/or co-stimulatory sequences in any order and of any combination of two to five intracellular sequences including 4-1BB, CD3zeta, CD28, CD27, OX40, CD30, CD40, PD-1, ICOS, lymphocyte function-associated antigen-1 (LFA-1), CD2, CD7, LIGHT, NKG2C, B7-H3, GITR (TNFRSF18), DR3 (TNFRSF25), TNFR2 and/or CD226. In one embodiment, the intracellular region comprises sequences from any one or any combination of two or more of CD28, 4-1BB and/or CD3-zeta. In one embodiment, the intracellular region comprises CD28 and CD3-zeta intracellular sequences, or 4-1BB and CD3-zeta intracellular sequences. In one embodiment, the CD3-zeta portion of the intracellular region comprises ITAM (immunoreceptor tyrosine-based activation motif) motifs 1, 2 and 3 (e.g., long CD3-zeta). In one embodiment, the CD3-zeta portion of the intracellular region comprises only one of the ITAM motifs such as only ITAM 1, 2 or 3 (e.g., short CD3-zeta).

The present disclosure provides a precursor polypeptide, comprising a plurality of regions ordered from the amino terminus to the carboxyl terminus: (i) a heavy chain leader sequence comprising the amino acid sequence of SEQ ID NO:54 or 56; (ii) a BCMA antibody heavy chain variable region comprising an amino acid sequence having at least 95% sequence identity to the amino acid sequence of any one of SEQ ID NOS:6, 12, 14, 16, 18, 20, 22, 24, 26, or 28; (iii) a BCMA antibody heavy chain constant region comprising the amino acid sequence of SEQ ID NO:7 or 29; (iv) a CD28 hinge region comprising the amino acid sequence of SEQ ID NO:35 and optionally a CD8 hinge comprising the amino acid sequence of SEQ ID NO:34; (v) a transmembrane region comprising the amino acid sequence of the amino acid sequence of SEQ ID NO:37 (from CD28), SEQ ID NO:38 (from CD8), SEQ ID NO:39 (from 4-1BB), or SEQ ID NO:40 (from CD3zeta); (vi) an intracellular region comprising any one or any combination of two or more intracellular sequences selected from a group consisting of SEQ ID NO:41 (from 4-1BB), SEQ ID NO:42 (from CD28), SEQ ID NO:43 (from OX40), SEQ ID NO:44 (CD3zeta ITAM 1, 2 and 3), SEQ ID NO:45 (CD3zeta ITAM 1), SEQ ID NO:46 (CD3zeta ITAM 2) and/or SEQ ID NO:47 (CD3zeta ITAM 3); (vii) a self-cleaving sequence comprising any one of the amino acid sequence of SEQ ID NO:57, 58, 59 or 60; (viii) a light chain leader sequence comprising the amino acid sequence of SEQ ID NO:55 or 56; (ix) a BCMA antibody light chain variable region comprising an amino acid sequence having at least 95% sequence identity to the amino acid sequence of any one of SEQ ID NO:8, 9, 10, 13, 15, 17, 19, 21, 23, 25, 27 or 30; and (x) a BCMA antibody light chain constant region comprising the amino acid sequence of SEQ ID NO:11 or 31. In one embodiment, the full length precursor polypeptide comprises the amino acid sequence of any one of SEQ ID NO:63, 66, 69, 72, 75, 78, 81 or 84. In one embodiment, the precursor polypeptide can be processed by cleaving at the self-cleaving sequence to release the first and second polypeptide chains and secreting the precursor, and/or anchoring the precursor in a cellular membrane. The first and second polypeptide chains can dimerize via at least one disulfide bond between the antibody heavy chain constant region and the antibody light chain constant region, and the antibody heavy chain variable region and the antibody light chain variable region can form an antigen binding domain that binds a BCMA antigen. In one embodiment, the precursor polypeptide lacks a hinge region.

The present disclosure provides a precursor polypeptide, comprising a plurality of regions ordered from the amino terminus to the carboxyl terminus: (i) a light chain leader sequence comprising the amino acid sequence of SEQ ID NO:55 or 56; (ii) a BCMA antibody light chain variable region comprising an amino acid sequence having at least 95% sequence identity to the amino acid sequence of any one of SEQ ID NO:8, 9, 10, 13, 15, 17, 19, 21, 23, 25, 27 or 30; (iii) a BCMA antibody light chain constant region comprising the amino acid sequence of SEQ ID NO:11 or 31; (iv) a CD28 hinge region comprising the amino acid sequence of SEQ ID NO:35 and optionally a CD8 hinge comprising the amino acid sequence of SEQ ID NO:34; (v) a transmembrane region comprising the amino acid sequence of SEQ ID NO:37 (from CD28), SEQ ID NO:38 (from CD8), SEQ ID NO:39 (from 4-1BB), or SEQ ID NO:40 (from CD3zeta); (vi) an intracellular region comprising any one or any combination of two or more intracellular sequences selected from a group consisting of SEQ ID NO:41 (from 4-1BB), SEQ ID NO:42 (from CD28), SEQ ID NO:43 (from OX40), SEQ ID NO:44 (CD3zeta ITAM 1, 2 and 3), SEQ ID NO:45 (CD3zeta ITAM 1), SEQ ID NO:46 (CD3zeta ITAM 2) and/or SEQ ID NO:47 (CD3zeta ITAM 3); (vii) a self-cleaving sequence comprising any one of the amino acid sequence of SEQ ID NO:57, 58, 59 or 60; (viii) a heavy chain leader sequence comprising the amino acid sequence of SEQ ID NO:54 or 56; (ix) a BCMA antibody heavy chain variable region comprising an amino acid sequence having at least 95% sequence identity to the amino acid sequence of any one of SEQ ID NOS:6, 12, 14, 16, 18, 20, 22, 24, 26, or 28; and (x) a BCMA antibody heavy chain constant region comprising the amino acid sequence of SEQ ID NO:7 or 29. In one embodiment, the precursor polypeptide can be processed by cleaving at the self-cleaving sequence to release the first and second polypeptide chains and secreting the precursor, and/or anchoring the precursor in a cellular membrane. The first and second polypeptide chains can dimerize via at least one disulfide bond between the antibody heavy chain constant region and the antibody light chain constant region, and the antibody heavy chain variable region and the antibody light chain variable region can form an antigen binding domain that binds a BCMA antigen. In one embodiment, the precursor polypeptide lacks a hinge region.

### Nucleic Acids Encoding Dimeric Antigen Receptors and Related Molecules

The present disclosure provides nucleic acids that encode any of the first polypeptide chains, second polypeptide chains, first and second polypeptide chains, dimeric antigen receptors or precursor polypeptides described herein. In any of the nucleic acid embodiments described herein that encode a precursor polypeptide comprising a self-cleaving sequence, the self-cleaving sequence may be a T2A, P2A, E2A, or F2A sequence. In some embodiments, the self-cleaving sequence is other than a T2A sequence, e.g., the self-cleaving sequence is a P2A, E2A, or F2A sequence.

The present disclosure provides a nucleic acid encoding a precursor polypeptide comprising: a plurality of regions ordered from the amino terminus to the carboxyl terminus: (i) a heavy chain leader region, (ii) an antibody heavy chain variable region, (iii) an antibody heavy chain constant region, (iv) a hinge region, (v) a transmembrane region, (vi) an intracellular region having two to five intracellular sequences, (vii) a self-cleaving sequence region, (viii) a light chain leader region, (ix) an antibody light chain variable region (e.g., kappa or lambda), and (x) an antibody light chain constant region. In one embodiment, the nucleic acid encodes a precursor polypeptide exemplified in FIG. 3A or B. In one embodiment, the nucleic acid encoding the precursor polypeptide lacks a hinge region. In one embodiment, the nucleic acid encoding the precursor polypeptide lacks a heavy chain leader region and/or a light chain leader region.

The present disclosure provides a nucleic acid encoding a precursor polypeptide comprising: a plurality of regions ordered from the amino terminus to the carboxyl terminus: (i) a heavy chain leader sequence comprising the amino acid sequence of SEQ ID NO:54 or 56; (ii) a BCMA antibody heavy chain variable region comprising an amino acid sequence having at least 95% sequence identity to the amino acid sequence of any one of SEQ ID NOS:6, 12, 14, 16, 18, 20, 22, 24, 26, or 28; (iii) a BCMA antibody heavy chain constant region comprising the amino acid sequence of SEQ ID NO:7 or 29; (iv) a CD28 hinge region comprising the amino acid sequence of SEQ ID NO:35 and optionally a CD8 hinge comprising the amino acid sequence of SEQ ID NO:34; (v) a transmembrane region comprising the amino acid sequence of the amino acid sequence of SEQ ID NO:37 (from CD28), SEQ ID NO:38 (from CD8), SEQ ID NO:39 (from 4-1BB), or SEQ ID NO:40 (from CD3zeta); (vi) an intracellular region comprising any one or any combination of two or more intracellular sequences selected from a group consisting of SEQ ID NO:41 (from 4-1BB), SEQ ID NO:42 (from CD28), SEQ ID NO:43 (from OX40), SEQ ID NO:44 (CD3zeta ITAM 1, 2 and 3), SEQ ID NO:45 (CD3zeta ITAM 1), SEQ ID NO:46 (CD3zeta ITAM 2) and/or SEQ ID NO:47 (CD3zeta ITAM 3); (vii) a self-cleaving sequence comprising any one of the amino acid sequence of SEQ ID NO:57, 58, 59 or 60; (viii) a light chain leader sequence comprising the amino acid sequence of SEQ ID NO:55 or 56; (ix) a BCMA antibody light chain variable region comprising an amino acid sequence having at least 95% sequence identity to the amino acid sequence of any one of SEQ ID NO:8, 9, 10, 13, 15, 17, 19, 21, 23, 25, 27 or 30; and (lx a BCMA antibody light chain constant region comprising the amino acid sequence of SEQ ID NO:11 or 31. In one embodiment, the nucleic acid encodes a precursor polypeptide exemplified in FIG. 3A or B. In one embodiment, the nucleic acid encoding the precursor polypeptide lacks a hinge region. In one embodiment, the nucleic acid encoding the precursor polypeptide lacks a heavy chain leader region and/or a light chain leader region.

The present disclosure provides a nucleic acid encoding a precursor polypeptide comprising: a plurality of regions ordered from the amino terminus to the carboxyl terminus: (i) a light chain leader region, (ii) an antibody light chain variable region (e.g., kappa or lambda), (iii) an antibody light chain constant region, (iv) a hinge region, (v) a transmembrane region, (vi) an intracellular region having two to five intracellular sequences, (vii) a self-cleaving sequence region, (viii) a heavy chain leader region, (ix) an antibody heavy chain variable region, and (x) an antibody heavy chain constant region. In one embodiment, the nucleic acid encodes a precursor polypeptide exemplified in FIG. 4A or B. In one embodiment, the nucleic acid encoding the precursor polypeptide lacks a hinge region. In one embodiment, the nucleic acid encoding the precursor polypeptide lacks a heavy chain leader region and/or a light chain leader region.

The present disclosure provides a nucleic acid encoding a precursor polypeptide comprising: a plurality of regions ordered from the amino terminus to the carboxyl terminus: (i) a light chain leader sequence comprising the amino acid sequence of SEQ ID NO:55 or 56; (ii) a BCMA antibody light chain variable region comprising an amino acid sequence having at least 95% sequence identity to the amino acid sequence of any one of SEQ ID NO:8, 9, 10, 13, 15, 17, 19, 21, 23, 25, 27 or 30; (iii) a BCMA antibody light chain constant region comprising the amino acid sequence of SEQ ID NO:11 or 31; (iv) a CD28 hinge region comprising the amino acid sequence of SEQ ID NO:35 and optionally a CD8 hinge comprising the amino acid sequence of SEQ ID NO:34; (v) a transmembrane region comprising the amino acid sequence of SEQ ID NO:37 (from CD28), SEQ ID NO:38 (from CD8), SEQ ID NO:39 (from 4-1BB), or SEQ ID NO:40 (from CD3zeta); (vi) an intracellular region comprising any one or any combination of two or more intracellular sequences selected from a group consisting of SEQ ID NO:41 (from 4-1BB), SEQ ID NO:42 (from CD28), SEQ ID NO:43 (from OX40), SEQ ID NO:44 (CD3zeta ITAM 1, 2 and 3), SEQ ID NO:45 (CD3zeta ITAM 1), SEQ ID NO:46 (CD3zeta ITAM 2) and/or SEQ ID NO:47 (CD3zeta ITAM 3); (vii) a self-cleaving sequence comprising any one of the amino acid sequence of SEQ ID NO:57, 58, 59 or 60; (viii) a heavy chain leader sequence comprising the amino acid sequence of SEQ ID NO:54 or 56; (ix) a BCMA antibody heavy chain variable region comprising an amino acid sequence having at least 95% sequence identity to the amino acid sequence of any one of SEQ ID NOS:6, 12, 14, 16, 18, 20, 22, 24, 26, or 28; and (x) a BCMA antibody heavy chain constant region comprising the amino acid sequence of SEQ ID NO:7 or 29. In one embodiment, the nucleic acid encodes a precursor polypeptide exemplified in FIG. 4A or B. In one embodiment, the nucleic acid encoding the precursor polypeptide lacks a hinge region. In one embodiment, the nucleic acid encoding the precursor polypeptide lacks a heavy chain leader region and/or a light chain leader region.

The present disclosure provides a first nucleic acid that encodes a first polypeptide chain comprising a plurality of regions ordered from the amino terminus to the carboxyl terminus: (i) a heavy chain leader region, (ii) an antibody heavy chain variable region, (iii) an antibody heavy chain constant region, (iv) a hinge region, (v) a transmembrane region, and (vi) an intracellular region. In one embodiment, a first nucleic acid encodes a first polypeptide that lacks a heavy chain leader region. In one embodiment, a first nucleic acid encodes a first polypeptide that lacks a hinge region. In one embodiment, a first nucleic acid encodes a first polypeptide chain exemplified in FIG. 1A or B.

The present disclosure provides a second nucleic acid that encodes a second polypeptide chain comprising a plurality of regions ordered from the amino terminus to the carboxyl terminus: (i) a light chain leader region, (ii) an antibody light chain variable region (e.g., kappa or lambda), and (iii) an antibody light chain constant region. In one embodiment, a second nucleic acid encodes a second polypeptide that lacks a light chain leader region. In one embodiment, a second nucleic acid encodes a second polypeptide chain exemplified in FIG. 1A or B.

In one embodiment, a nucleic acid encodes first and second polypeptide chains, comprising: (a) a first polypeptide chain comprising a plurality of regions ordered from the amino terminus to the carboxyl terminus: (i) a heavy chain leader sequence, (ii) an antibody heavy chain variable region, (iii) an antibody heavy chain constant region, (iv) a hinge region, (v) a transmembrane region, and (vi) an intracellular region having two to five intracellular sequences; and (b) a second polypeptide chain comprising: a plurality of regions ordered from the amino terminus to the carboxyl terminus: (i) an antibody light chain variable region (e.g., kappa or lambda), and (ii) an antibody light chain constant region. In one embodiment, a single nucleic acid encodes a first polypeptide that lacks a heavy chain leader region and/or a single nucleic acid encodes a second polypeptide that lacks a light chain leader region. In one embodiment, a single nucleic acid encodes a first polypeptide that lacks a hinge region. In one embodiment, a single nucleic acid encodes first and second polypeptide chains exemplified in FIG. 1A or B.

The present disclosure provides a first nucleic acid that encodes a first polypeptide chain comprising a plurality of regions ordered from the amino terminus to the carboxyl terminus: (i) a light chain leader region, (ii) an antibody light chain variable region (e.g., kappa or lambda), (iii) an antibody light chain constant region, (iv) a hinge region, (v) a transmembrane region, and (vi) an intracellular region. In one embodiment, a first nucleic acid encodes a first polypeptide that lacks a light chain leader region. In one embodiment, a first nucleic acid encodes a first polypeptide that lacks a hinge region. In one embodiment, a first nucleic acid encodes a first polypeptide chain exemplified in FIG. 2A or B.

The present disclosure provides a second nucleic acid that encodes a second polypeptide chain comprising a plurality of regions ordered from the amino terminus to the carboxyl terminus: (i) a heavy chain leader region, (ii) an antibody heavy chain variable region, and (iii) an antibody light chain constant region. In one embodiment, a second nucleic acid encodes a second polypeptide that lacks a heavy chain leader region. In one embodiment, a second nucleic acid encodes a second polypeptide chain exemplified in FIG. 2A or B.

In one embodiment, a nucleic acid encodes first and second polypeptide chains, comprising: (a) a first polypeptide chain comprising a plurality of regions ordered from the amino terminus to the carboxyl terminus: (i) a light chain leader sequence, (ii) an antibody light chain variable region (e.g., kappa or lambda), (iii) an antibody light chain constant region, (iv) a hinge region, (v) a transmembrane region, and (vi) an intracellular region having two to five intracellular sequences; and (b) a second polypeptide chain comprising: a plurality of regions ordered from the amino terminus to the carboxyl terminus: (i) an antibody heavy chain variable region, and (ii) an antibody heavy chain constant region. In one embodiment, a single nucleic acid encodes a first polypeptide that lacks a light chain leader region and/or a single nucleic acid encodes a second polypeptide that lacks a heavy chain leader region. In one embodiment, a single nucleic acid encodes a first polypeptide that lacks a hinge region. In one embodiment, a single nucleic acid encodes first and second polypeptide chains exemplified in FIG. 2A or B.

The present disclosure provides nucleic acids that encode a first polypeptide chain comprising a plurality of regions ordered from the amino terminus to the carboxyl terminus: (i) a BCMA antibody heavy chain variable region comprising an amino acid sequence having at least 95% sequence identity to the amino acid sequence of any one of SEQ ID NOS:6, 12, 14, 16, 18, 20, 22, 24, 26, or 28; (ii) a BCMA antibody heavy chain constant region comprising the amino acid sequence of SEQ ID NO:7 or 29; (iii) a CD28 hinge region comprising the amino acid sequence of SEQ ID NO:35 and optionally a CD8 hinge comprising the amino acid sequence of SEQ ID NO:34; (iv) a transmembrane region comprising the amino acid sequence of SEQ ID NO:37 (from CD28), SEQ ID NO:38 (from CD8), SEQ ID NO:39 (from 4-1BB), or SEQ ID NO:40 (from CD3zeta); and (v) an intracellular region comprising the amino acid sequence from any one or any combination of two or more intracellular sequences selected from a group consisting of SEQ ID NO:41 (from 4-1BB), SEQ ID NO:42 (from CD28), SEQ ID NO:43 (from OX40), SEQ ID NO:44 (CD3zeta ITAM 1, 2 and 3), SEQ ID NO:45 (CD3zeta ITAM 1), SEQ ID NO:46 (CD3zeta ITAM 2) and/or SEQ ID NO:47 (CD3zeta ITAM 3). In one embodiment, the nucleic acid encodes a first polypeptide chain which comprises the amino acid sequence of any one of SEQ ID NO:64, 67, 70, 73, 76, 79, 82 or 85, wherein the first polypeptide chains includes or lacks a leader sequence (e.g., SEQ ID NO:54 OR 55). In one embodiment, the nucleic acid encodes a first polypeptide chain lacking a hinge region.

The present disclosure provide nucleic acids that encode a second polypeptide chain comprising a plurality of regions ordered from the amino terminus to the carboxyl terminus: (i) a BCMA antibody light chain variable region comprising an amino acid sequence having at least 95% sequence identity to the amino acid sequence of any one of SEQ ID NOS:6, 12, 14, 16, 18, 20, 22, 24, 26, or 28, and (ii) a BCMA antibody light chain constant region comprising an amino acid sequence having at least 95% sequence identity to the amino acid sequence of any one of SEQ ID NO:8, 9, 10, 13, 15, 17, 19, 21, 23, 25, 27 or 30. In one embodiment, the nucleic acid encodes a second polypeptide chain which comprises the amino acid sequence of any one of SEQ ID NO:65, 68, 71, 74, 77, 80, 83 or 86, wherein the second polypeptide chains includes or lacks a leader sequence (e.g., SEQ ID NO:55 or 56).

The present disclosure provides nucleic acids that encode a Version 1 (e.g., V1) dimeric antigen receptors (DAR) construct comprising a first polypeptide chain carrying heavy chain variable (VH) and heavy chain constant regions (CH), and a second polypeptide chain carrying light chain variable (VL) (e.g., kappa or lambda) and light chain constant regions (CL) (e.g., FIG. 1A), wherein (a) a first nucleic acid encodes the first polypeptide chain comprising a plurality of regions ordered from the amino terminus to the carboxyl terminus: (i) an antibody heavy chain variable region (VH); (ii) an antibody heavy chain constant region (CH); (iii) a long hinge region comprising CD8 and CD28 hinge sequences (e.g., SEQ ID NO:36); (iv) a transmembrane region (TM) comprising CD28 transmembrane sequence (e.g., SEQ ID NO:37); and (v) an intracellular region comprising CD28 co-stimulatory sequence (e.g., SEQ ID NO:42) and CD3-zeta signaling sequence having ITAM motifs 1, 2 and 3 (e.g., SEQ ID NO:44); (b) the second nucleic acid encodes a second polypeptide chain comprising a plurality of regions ordered from the amino terminus to the carboxyl terminus: (i) an antibody light chain variable region (VL) (e.g., kappa or lambda), and (ii) an antibody light chain constant region (CL). In one embodiment, the antibody heavy chain variable region (VH) comprises an anti-BCMA heavy chain variable region sequence having an amino acid sequence with at least 95% sequence identity to the amino acid sequence of any one of SEQ ID NOS:6, 12, 14, 16, 18, 20, 22, 24, 26, or 28. In one embodiment, the antibody light chain variable region (VL) comprises an anti-BCMA light chain variable region sequence having an amino acid sequence with at least 95% sequence identity to the amino acid sequence of any one of SEQ ID NOS:8, 9, 10, 13, 15, 17, 19, 21, 23, 25, 27 or 30.

The present disclosure provides nucleic acids that encode a Version 2 (e.g., V2) dimeric antigen receptors (DAR) construct comprising a first polypeptide chain carrying heavy chain variable (VH) and heavy chain constant regions (CH), and a second polypeptide chain carrying light chain variable (VL) and light chain constant regions (CL) (e.g., FIG. 1A or B), wherein (a) a first nucleic acid encodes the first polypeptide chain comprising a plurality of regions ordered from the amino terminus to the carboxyl terminus: (i) an antibody heavy chain variable region (VH), (ii) an antibody heavy chain constant region (CH), (iii) a short hinge region comprising a CD28 hinge sequence (e.g., SEQ ID NO:37), (iv) a transmembrane region (TM) comprising CD28 transmembrane sequence (e.g., SEQ ID NO:37), and (v) an intracellular region comprising either (1) a 4-1BB co-stimulatory sequence (e.g., SEQ ID NO:41) and CD3-zeta signaling sequence having ITAM motifs 1, 2 and 3 (e.g., SEQ ID NO:44), or (2) CD28 co-stimulatory sequence (e.g., SEQ ID NO:42) and CD3-zeta signaling sequence having ITAM motifs 1, 2 and 3 (e.g., SEQ ID NO:44), or (3) 4-1BB co-stimulatory sequence (e.g., SEQ ID NO:41) and CD28 co-stimulatory sequence (e.g., SEQ ID NO:42) and CD3-zeta signaling sequence having ITAM motifs 1, 2 and 3 (e.g., SEQ ID NO:44); (b) a second nucleic acid encodes the second polypeptide chain comprising a plurality of regions ordered from the amino terminus to the carboxyl terminus: (i) an antibody light chain variable region (VL) (e.g., kappa or lambda), and (ii) an antibody light chain constant region (CL).

In one embodiment, the nucleic acids encode the Version 2a (V2a) DAR construct comprising the intracellular region having the 4-1BB co-stimulatory sequence (e.g., SEQ ID NO:41) and CD3-zeta signaling sequence having ITAM motifs 1, 2 and 3 (e.g., SEQ ID NO:44).

In one embodiment, the nucleic acids encode the Version 2b (V2b) DAR construct comprising the intracellular region having the CD28 co-stimulatory sequence (e.g., SEQ ID NO:42) and CD3-zeta signaling sequence having ITAM motifs 1, 2 and 3 (e.g., SEQ ID NO:44).

In one embodiment, the nucleic acids encode the Version 2c (V2c) DAR construct comprising the intracellular region having the 4-1BB co-stimulatory region (e.g., SEQ ID NO:41) and CD28 co-stimulatory region (e.g., SEQ ID NO:42) and CD3-zeta signaling sequence having ITAM motifs 1, 2 and 3 (e.g., SEQ ID NO:44). In one embodiment, the DAR V2a and V2b are second generation DAR constructs, while the DAR V2c is a third generation DAR construct. In one embodiment, the antibody heavy chain variable region (VH) comprises an anti-BCMA heavy chain variable region sequence having an amino acid sequence with at least 95% sequence identity to the amino acid sequence of any one of SEQ ID NOS:6, 12, 14, 16, 18, 20, 22, 24, 26, or 28. In one embodiment, the antibody light chain variable region (VL) comprises an anti-BCMA light chain variable region sequence having an amino acid sequence with at least 95% sequence identity to the amino acid sequence of any one of SEQ ID NOS:8, 9, 10, 13, 15, 17, 19, 21, 23, 25, 27 or 30.

The present disclosure provides nucleic acids that encodes a Version 3a (e.g., V3a) dimeric antigen receptors (DAR) construct comprising a first polypeptide chain carrying heavy chain variable (VH) and heavy chain constant regions (CH), and a second polypeptide chain carrying light chain variable (VL) and light chain constant regions (CL) (e.g., FIG. 1A), wherein (a) a first nucleic acid encodes the first polypeptide chain comprising a plurality of regions ordered from the amino terminus to the carboxyl terminus: (i) an antibody heavy chain variable region (VH), (ii) an antibody heavy chain constant region (CH), (iii) a short hinge region comprising CD28 hinge sequences (e.g., SEQ ID NO:35), (iv) a transmembrane region (TM) comprising CD28 transmembrane sequence (e.g., SEQ ID NO:37), and (v) an intracellular region comprising either (1) 4-1BB co-stimulatory sequence (e.g., SEQ ID NO:41) and CD3-zeta signaling sequence having ITAM motif 3 (e.g., SEQ ID NO:47), or (2) CD28 (e.g., SEQ ID NO:42) signaling sequence and CD3-zeta having ITAM motif 3 (e.g., SEQ ID NO:47), or (3) 4-1BB (e.g., SEQ ID NO:41) signaling sequence and CD28 (e.g., SEQ ID NO:42) signaling sequence and CD3-zeta having ITAM motif 3 (e.g., SEQ ID NO:47); (b) a second nucleic acid encodes the second polypeptide chain comprising a plurality of regions ordered from the amino terminus to the carboxyl terminus: (i) an antibody light chain variable region (VL) (e.g., kappa or lambda), and (ii) an antibody light chain constant region (CL). In one embodiment, the antibody heavy chain variable region (VH) comprises an anti-BCMA heavy chain variable region sequence having an amino acid sequence with at least 95% sequence identity to the amino acid sequence of any one of SEQ ID NOS:6, 12, 14, 16, 18, 20, 22, 24, 26, or 28. In one embodiment, the antibody light chain variable region (VL) comprises an anti-BCMA light chain variable region sequence having an amino acid sequence with at least 95% sequence identity to the amino acid sequence of any one of SEQ ID NOS:8, 9, 10, 13, 15, 17, 19, 21, 23, 25, 27 or 30.

In one embodiment, the nucleic acids encode the Version 3a (V3a) DAR construct comprising the intracellular region having the 4-1BB co-stimulatory sequence (e.g., SEQ ID NO:41) and CD3-zeta having ITAM motif 3 (e.g., SEQ ID NO:47).

In one embodiment, the nucleic acids encode the Version 3b (V3b) DAR construct comprises the intracellular region having the CD28 (e.g., SEQ ID NO:42) signaling sequence and CD3-zeta having ITAM motif 3 (e.g., SEQ ID NO:47).

In one embodiment, the nucleic acids encode the Version 3c (V3c) DAR construct comprises the intracellular region having the 4-1BB (e.g., SEQ ID NO:41) signaling sequence and CD28 (e.g., SEQ ID NO:42) signaling sequence and CD3-zeta having ITAM motif 3 (e.g., SEQ ID NO:47).

In one embodiment, the DAR V3a and V3b are second generation DAR constructs, while the DAR V3c is a third generation DAR construct.

In one embodiment, in the DAR V3a, V3b and V3c constructs, the antibody heavy chain variable region (VH) comprises an anti-BCMA heavy chain variable region comprising an amino acid sequence having at least 95% sequence identity to the amino acid sequence of any one of SEQ ID NOS:6, 12, 14, 16, 18, 20, 22, 24, 26, or 28, and the antibody light chain variable region (VH) comprises an anti-BCMA light chain variable region sequence comprising an amino acid sequence having at least 95% sequence identity to the amino acid sequence of any one of SEQ ID NO: 8, 9, 10, 13, 15, 17, 19, 21, 23, 25, 27 or 30.

The present disclosure provides nucleic acids that encode a Version 4 (e.g., V4) dimeric antigen receptors (DAR) construct comprising a first polypeptide chain carrying heavy chain variable (VH) and heavy chain constant regions (CH), and a second polypeptide chain carrying light chain variable (VL) and light chain constant regions (CL) (e.g, FIG. 1A but without the hinge), wherein (a) a first nucleic acid encodes the first polypeptide chain comprising a plurality of regions ordered from the amino terminus to the carboxyl terminus: (i) an antibody heavy chain variable region (VH), (ii) an antibody heavy chain constant region (CH), (iii) a transmembrane region (TM) comprising CD28 transmembrane sequence (e.g., SEQ ID NO:37), and (iv) an intracellular region comprising 4-1BB co-stimulatory sequence (e.g., SEQ ID NO:41) and CD3-zeta signaling sequence having ITAM motif 3 (e.g., SEQ ID NO:47); (b) a second nucleic acid encodes the second polypeptide chain comprising a plurality of regions ordered from the amino terminus to the carboxyl terminus: (i) an antibody light chain variable region (VL) (e.g., kappa or lambda), and (ii) an antibody light chain constant region (CL). The DAR V4 construct lacks a hinge sequence. In one embodiment, the antibody heavy chain variable region (VH) comprises an anti-BCMA heavy chain variable region sequence having an amino acid sequence with at least 95% sequence identity to the amino acid sequence of any one of SEQ ID NOS:6, 12, 14, 16, 18, 20, 22, 24, 26, or 28. In one embodiment, the antibody light chain variable region (VL) comprises an anti-BCMA light chain variable region sequence having an amino acid sequence with at least 95% sequence identity to the amino acid sequence of any one of SEQ ID NOS:8, 9, 10, 13, 15, 17, 19, 21, 23, 25, 27 or 30.

### Vectors

The present disclosure provides vectors operably linked to one or more nucleic acids that encode any of the precursor polypeptides, first polypeptide chains, second polypeptide chains, or first and second polypeptide chains described herein.

The present disclosure provides a vector operably linked to a nucleic acid that encodes a precursor polypeptide comprising: a plurality of regions ordered from the amino terminus to the carboxyl terminus: (i) a heavy chain leader region, (ii) an antibody heavy chain variable region, (iii) an antibody heavy chain constant region, (iv) a hinge region, (v) a transmembrane region, (vi) an intracellular region having two to five intracellular sequences, (vii) a self-cleaving sequence region, (viii) a light chain leader region, (ix) an antibody light chain variable region, and (x) an antibody light chain constant region. In one embodiment, the precursor polypeptide lacks a hinge region. In one embodiment, the precursor polypeptide lacks a heavy chain leader region and/or a light chain leader region. In one embodiment, the precursor polypeptide is exemplified in FIG. 3A or B.

The present disclosure provides a vector operably linked to a nucleic acid that encodes a precursor polypeptide comprising: a plurality of regions ordered from the amino terminus to the carboxyl terminus: (i) a heavy chain leader sequence comprising the amino acid sequence of SEQ ID NO:54 or 56; (ii) a BCMA antibody heavy chain variable region comprising an amino acid sequence having at least 95% sequence identity to the amino acid sequence of any one of SEQ ID NOS:6, 12, 14, 16, 18, 20, 22, 24, 26, or 28; (iii) a BCMA antibody heavy chain constant region comprising the amino acid sequence of SEQ ID NO:7 or 29; (iv) a CD28 hinge region comprising the amino acid sequence of SEQ ID NO:35 and optionally a CD8 hinge comprising the amino acid sequence of SEQ ID NO:34; (v) a transmembrane region comprising the amino acid sequence of the amino acid sequence of SEQ ID NO:37 (from CD28), SEQ ID NO:38 (from CD8), SEQ ID NO:39 (from 4-1BB), or SEQ ID NO:40 (from CD3zeta); (vi) an intracellular region comprising any one or any combination of two or more intracellular sequences selected from a group consisting of SEQ ID NO:41 (from 4-1BB), SEQ ID NO:42 (from CD28), SEQ ID NO:43 (from OX40), SEQ ID NO:44 (CD3zeta ITAM 1, 2 and 3), SEQ ID NO:45 (CD3zeta ITAM 1), SEQ ID NO:46 (CD3zeta ITAM 2) and/or SEQ ID NO:47 (CD3zeta ITAM 3); (vii) a self-cleaving sequence comprising any one of the amino acid sequence of SEQ ID NO:57, 58, 59 or 60; (viii) a light chain leader sequence comprising the amino acid sequence of SEQ ID NO:55 or 56; (ix) a BCMA antibody light chain variable region comprising an amino acid sequence having at least 95% sequence identity to the amino acid sequence of any one of SEQ ID NO:8, 9, 10, 13, 15, 17, 19, 21, 23, 25, 27 or 30; and (x) a BCMA antibody light chain constant region comprising the amino acid sequence of SEQ ID NO:11 or 31. In one embodiment, the nucleic acid encodes a precursor polypeptide exemplified in FIG. 3A or B. In one embodiment, the nucleic acid encoding the precursor polypeptide lacks a hinge region. In one embodiment, the nucleic acid encoding the precursor polypeptide lacks a heavy chain leader region and/or a light chain leader region.

The present disclosure provides a vector operably linked to a nucleic acid that encodes a precursor polypeptide comprising: a plurality of regions ordered from the amino terminus to the carboxyl terminus: (i) a light chain leader region, (ii) an antibody light chain variable region, (iii) an antibody light chain constant region, (iv) a hinge region, (v) a transmembrane region, (vi) an intracellular region having two to five intracellular sequences, (vii) a self-cleaving sequence region, (viii) a heavy chain leader region, (ix) an antibody heavy chain variable region, and (x) an antibody heavy chain constant region. In one embodiment, the precursor polypeptide lacks a hinge region. In one embodiment, the precursor polypeptide lacks a heavy chain leader region and/or a light chain leader region. In one embodiment, the precursor polypeptide is exemplified in FIG. 4A or B.

The present disclosure provides a nucleic acid encoding a precursor polypeptide comprising: a plurality of regions ordered from the amino terminus to the carboxyl terminus: (i) a light chain leader sequence comprising the amino acid sequence of SEQ ID NO:55 or 56; (ii) a BCMA antibody light chain variable region comprising an amino acid sequence having at least 95% sequence identity to the amino acid sequence of any one of SEQ ID NO:8, 9, 10, 13, 15, 17, 19, 21, 23, 25, 27 or 30; (iii) a BCMA antibody light chain constant region comprising the amino acid sequence of SEQ ID NO:11 or 31; (iv) a CD28 hinge region comprising the amino acid sequence of SEQ ID NO:35 and optionally a CD8 hinge comprising the amino acid sequence of SEQ ID NO:34; (v) a transmembrane region comprising the amino acid sequence of SEQ ID NO:37 (from CD28), SEQ ID NO:38 (from CD8), SEQ ID NO:39 (from 4-1BB), or SEQ ID NO:40 (from CD3zeta); (vi) an intracellular region comprising any one or any combination of two or more intracellular sequences selected from a group consisting of SEQ ID NO:41 (from 4-1BB), SEQ ID NO:42 (from CD28), SEQ ID NO:43 (from OX40), SEQ ID NO:44 (CD3zeta ITAM 1, 2 and 3), SEQ ID NO:45 (CD3zeta ITAM 1), SEQ ID NO:46 (CD3zeta ITAM 2) and/or SEQ ID NO:47 (CD3zeta ITAM 3); (vii) a self-cleaving sequence comprising any one of the amino acid sequence of SEQ ID NO:57, 58, 59 or 60; (viii) a heavy chain leader sequence comprising the amino acid sequence of SEQ ID NO:54 or 56; (ix) a BCMA antibody heavy chain variable region comprising an amino acid sequence having at least 95% sequence identity to the amino acid sequence of any one of SEQ ID NOS:6, 12, 14, 16, 18, 20, 22, 24, 26, or 28; and (x) a BCMA antibody heavy chain constant region comprising the amino acid sequence of SEQ ID NO:7 or 29. In one embodiment, the nucleic acid encodes a precursor polypeptide exemplified in FIG. 4A or B. In one embodiment, the nucleic acid encoding the precursor polypeptide lacks a hinge region. In one embodiment, the nucleic acid encoding the precursor polypeptide lacks a heavy chain leader region and/or a light chain leader region.

The present disclosure provides a first vector operably linked to a first nucleic acid that encodes a first polypeptide chain comprising: a plurality of regions ordered from the amino terminus to the carboxyl terminus: (i) a heavy chain leader region, (ii) an antibody heavy chain variable region, (iii) an antibody heavy chain constant region, (iv) a hinge region, (v) a transmembrane region, and (vi) an intracellular region. In one embodiment, the first vector is operably linked to a first nucleic acid encoding a first polypeptide chain that lacks a heavy chain leader region and/or lacks a hinge region. In one embodiment, the first polypeptide chain is exemplified in FIG. 1A or B.

In one embodiment, the first vector is operably linked to a first nucleic acid that encodes a first polypeptide chain comprising: a plurality of regions ordered from the amino terminus to the carboxyl terminus: (i) a heavy chain leader sequence comprising the amino acid sequence of SEQ ID NO:54; (ii) a BCMA antibody heavy chain variable region comprising an amino acid sequence having at least 95% sequence identity to the amino acid sequence of any one of SEQ ID NOS: 6, 12, 14, 16, 18, 20, 22, 24, 26, or 28; (iii) a BCMA antibody heavy chain constant region comprising the amino acid sequence of SEQ ID NO:7 or 29; (iv) a CD28 hinge region comprising the amino acid sequence of SEQ ID NO:35 and optionally a CD8 hinge comprising the amino acid sequence of SEQ ID NO:34; (v) a transmembrane region comprising the amino acid sequence of the amino acid sequence of SEQ ID NO:37 (from CD28), SEQ ID NO:38 (from CD8), SEQ ID NO:39 (from 4-1BB), or SEQ ID NO:40 (from CD3zeta); (vi) an intracellular region comprising any one or any combination of two or more intracellular sequences selected from a group consisting of SEQ ID NO:41 (from 4-1BB), SEQ ID NO:42 (from CD28), SEQ ID NO:43 (from OX40), SEQ ID NO:44 (CD3zeta ITAM 1, 2 and 3), SEQ ID NO:45 (CD3zeta ITAM 1), SEQ ID NO:46 (CD3zeta ITAM 2) and/or SEQ ID NO:47 (CD3zeta ITAM 3). In one embodiment, the first vector is operably linked to a first nucleic acid encoding a first polypeptide chain that lacks a heavy chain leader region and/or lacks a hinge region. In one embodiment, the first polypeptide chain is exemplified in FIG. 1A or B.

The present disclosure provides a second vector operably linked to a second nucleic acid that encodes a second polypeptide chain comprising: a plurality of regions ordered from the amino terminus to the carboxyl terminus: (i) a light chain leader region, (ii) an antibody light chain variable region, and (iii) an antibody light chain constant region. In one embodiment, the second vector is operably linked to a second nucleic acid encoding a second polypeptide chain that lacks a light chain leader region. In one embodiment, the second polypeptide chain is exemplified in FIG. 1A or B.

In one embodiment, the second vector is operably linked to a second nucleic acid that encodes a second polypeptide chain comprising: a plurality of regions ordered from the amino terminus to the carboxyl terminus: (i) a light chain leader sequence comprising the amino acid sequence of SEQ ID NO:55; (ii) a BCMA antibody light chain variable region comprising an amino acid sequence having at least 95% sequence identity to the amino acid sequence of any one of SEQ ID NO: 8, 9, 10, 13, 15, 17, 19, 21, 23, 25, 27 or 30; and (iii) a BCMA antibody light chain constant region comprising the amino acid sequence of SEQ ID NO:11 or 31. In one embodiment, the second vector is operably linked to a second nucleic acid encoding a second polypeptide chain that lacks a light chain leader region. In one embodiment, the second polypeptide chain is exemplified in FIG. 1A or B.

The present disclosure provides a first vector operably linked to a first nucleic acid that encodes a first polypeptide chain comprising: a plurality of regions ordered from the amino terminus to the carboxyl terminus: (i) a light chain leader region, (ii) an antibody light chain variable region, (iii) an antibody light chain constant region, (iv) a hinge region, (v) a transmembrane region, and (vi) an intracellular region. In one embodiment, the first vector is operably linked to a first nucleic acid encoding a first polypeptide chain that lacks a heavy chain leader region and/or lacks a hinge region. In one embodiment, the first polypeptide chain is exemplified in FIG. 2A or B.

The present disclosure provides a first vector operably linked to a first nucleic acid that encodes a first polypeptide chain comprising: a plurality of regions ordered from the amino terminus to the carboxyl terminus: (i) a light chain leader sequence comprising the amino acid sequence of SEQ ID NO:55; (ii) a BCMA antibody light chain variable region comprising an amino acid sequence having at least 95% sequence identity to the amino acid sequence of any one of SEQ ID NOS: 8, 9, 10, 13, 15, 17, 19, 21, 23, 25, 27 or 30; (iii) a BCMA antibody light chain constant region comprising the amino acid sequence of SEQ ID NO:11 or 31; (iv) a CD28 hinge region comprising the amino acid sequence of SEQ ID NO:35 and optionally a CD8 hinge comprising the amino acid sequence of SEQ ID NO:34; (v) a transmembrane region comprising the amino acid sequence of the amino acid sequence of SEQ ID NO:37 (from CD28), SEQ ID NO:38 (from CD8), SEQ ID NO:39 (from 4-1BB), or SEQ ID NO:40 (from CD3zeta); (vi) an intracellular region comprising any one or any combination of two or more intracellular sequences selected from a group consisting of SEQ ID NO:41 (from 4-1BB), SEQ ID NO:42 (from CD28), SEQ ID NO:43 (from OX40), SEQ ID NO:44 (CD3zeta ITAM 1, 2 and 3), SEQ ID NO:45 (CD3zeta ITAM 1), SEQ ID NO:46 (CD3zeta ITAM 2) and/or SEQ ID NO:47 (CD3zeta ITAM 3). In one embodiment, the first vector is operably linked to a first nucleic acid encoding a first polypeptide chain that lacks a light chain leader region and/or lacks a hinge region. In one embodiment, the first polypeptide chain is exemplified in FIG. 2A or B.

The present disclosure provides a second vector operably linked to a second nucleic acid that encodes a second polypeptide chain comprising: a plurality of regions ordered from the amino terminus to the carboxyl terminus: (i) a heavy chain leader region, (ii) an antibody heavy chain variable region, and (iii) an antibody light chain constant region. In one embodiment, the second vector is operably linked to a second nucleic acid encoding a second polypeptide chain that lacks a heavy chain leader region. In one embodiment, the second polypeptide chain is exemplified in FIG. 2A or B.

The present disclosure provides a second vector operably linked to a second nucleic acid that encodes a second polypeptide chain comprising: a plurality of regions ordered from the amino terminus to the carboxyl terminus: (i) a heavy chain leader sequence comprising the amino acid sequence of SEQ ID NO:54; (ii) a BCMA antibody heavy chain variable region comprising an amino acid sequence having at least 95% sequence identity to the amino acid sequence of any one of SEQ ID NO: 6, 12, 14, 16, 18, 20, 22, 24, 26, or 28; and (iii) a BCMA antibody heavy chain constant region comprising the amino acid sequence of SEQ ID NO:7 or 29. In one embodiment, the second vector is operably linked to a second nucleic acid encoding a second polypeptide chain that lacks a heavy chain leader region. In one embodiment, the second polypeptide chain is exemplified in FIG. 2A or B.

The present disclosure provides a vector that is operably linked to nucleic acids encoding the first and second polypeptide chains wherein: (a) the first nucleic acid encodes the first polypeptide chain which comprises a plurality of regions ordered from the amino terminus to the carboxyl terminus: (i) a heavy chain leader sequence, (ii) an antibody heavy chain variable region, (iii) an antibody heavy chain constant region, (iv) a hinge region, (v) a transmembrane region, and (vi) an intracellular region having two to five intracellular sequences; and (b) the second nucleic acid encodes the second polypeptide chain which comprises: a plurality of regions ordered from the amino terminus to the carboxyl terminus: (i) an antibody light chain variable region, and (ii) an antibody light chain constant region. In one embodiment, the first nucleic acid encodes the first polypeptide chain that lacks a heavy chain leader region and/or lacks a hinge region. In one embodiment, the second nucleic acid encodes the second polypeptide chain that lacks a light chain leader region. In one embodiment, the first and second polypeptide chains are exemplified in FIG. 1A or B.

The present disclosure provides a vector that is operably linked to nucleic acids encoding first and second polypeptide chains wherein: (a) the first nucleic acid encodes the first polypeptide which comprises a plurality of regions ordered from the amino terminus to the carboxyl terminus: (i) a heavy chain leader sequence comprising the amino acid sequence of SEQ ID NO:54; (ii) a BCMA antibody heavy chain variable region comprising an amino acid sequence having at least 95% sequence identity to the amino acid sequence of any one of SEQ ID NOS: 6, 12, 14, 16, 18, 20, 22, 24, 26, or 28; (iii) a BCMA antibody heavy chain constant region comprising the amino acid sequence of SEQ ID NO:7 or 29; (iv) a CD28 hinge region comprising the amino acid sequence of SEQ ID NO:35 and optionally a CD8 hinge comprising the amino acid sequence of SEQ ID NO:34; (v) a transmembrane region comprising the amino acid sequence of the amino acid sequence of SEQ ID NO:37 (from CD28), SEQ ID NO:38 (from CD8), SEQ ID NO:39 (from 4-1BB), or SEQ ID NO:40 (from CD3zeta); (vi) an intracellular region comprising any one or any combination of two or more intracellular sequences selected from a group consisting of SEQ ID NO:41 (from 4-1BB), SEQ ID NO:42 (from CD28), SEQ ID NO:43 (from OX40), SEQ ID NO:44 (CD3zeta ITAM 1, 2 and 3), SEQ ID NO:45 (CD3zeta ITAM 1), SEQ ID NO:46 (CD3zeta ITAM 2) and/or SEQ ID NO:47 (CD3zeta ITAM 3); and (b) the second nucleic acid encodes the second polypeptide which comprises a plurality of regions ordered from the amino terminus to the carboxyl terminus: (i) a light chain leader sequence comprising the amino acid sequence of SEQ ID NO:55; (ii) a BCMA antibody light chain variable region comprising an amino acid sequence having at least 95% sequence identity to the amino acid sequence of any one of SEQ ID NO: 8, 9, 10, 13, 15, 17, 19, 21, 23, 25, 27 or 30; and (iii) a BCMA antibody light chain constant region comprising the amino acid sequence of SEQ ID NO:11 or 31. In one embodiment, the first nucleic acid encodes the first polypeptide chain that lacks a heavy chain leader region and/or lacks a hinge region. In one embodiment, the second nucleic acid encodes the second polypeptide chain that lacks a light chain leader region. In one embodiment, the first and second polypeptide chains are exemplified in FIG. 1A or B.

The present disclosure provides a vector that is operably linked to nucleic acids encoding the first and second polypeptide chains wherein: (a) the first nucleic acid encodes the first polypeptide chain which comprises a plurality of regions ordered from the amino terminus to the carboxyl terminus: (i) a light chain leader sequence, (ii) an antibody light chain variable region, (iii) an antibody light chain constant region, (iv) a hinge region, (v) a transmembrane region, and (vi) an intracellular region having two to five intracellular sequences; and (b) the second nucleic acid encodes the second polypeptide chain which comprises: a plurality of regions ordered from the amino terminus to the carboxyl terminus: (i) an antibody heavy chain variable region, and (ii) an antibody heavy chain constant region. In one embodiment, the first nucleic acid encodes the first polypeptide chain that lacks a light chain leader region and/or lacks a hinge region. In one embodiment, the second nucleic acid encodes the second polypeptide chain that lacks a heavy chain leader region. In one embodiment, the first and second polypeptide chains are exemplified in FIG. 2A or B.

The present disclosure provides a vector that is operably linked to nucleic acids encoding first and second polypeptide chains wherein: (a) the first nucleic acid encodes the first polypeptide which comprises a plurality of regions ordered from the amino terminus to the carboxyl terminus: (i) a light chain leader sequence comprising the amino acid sequence of SEQ ID NO:55; (ii) a BCMA antibody light chain variable region comprising an amino acid sequence having at least 95% sequence identity to the amino acid sequence of any one of SEQ ID NOS: 8, 9, 10, 13, 15, 17, 19, 21, 23, 25, 27 or 30; (iii) a BCMA antibody light chain constant region comprising the amino acid sequence of SEQ ID NO:11 or 31; (iv) a CD28 hinge region comprising the amino acid sequence of SEQ ID NO:35 and optionally a CD8 hinge comprising the amino acid sequence of SEQ ID NO:34; (v) a transmembrane region comprising the amino acid sequence of the amino acid sequence of SEQ ID NO:37 (from CD28), SEQ ID NO:38 (from CD8), SEQ ID NO:39 (from 4-1BB), or SEQ ID NO:40 (from CD3zeta); (vi) an intracellular region comprising any one or any combination of two or more intracellular sequences selected from a group consisting of SEQ ID NO:41 (from 4-1BB), SEQ ID NO:42 (from CD28), SEQ ID NO:43 (from OX40), SEQ ID NO:44 (CD3zeta ITAM 1, 2 and 3), SEQ ID NO:45 (CD3zeta ITAM 1), SEQ ID NO:46 (CD3zeta ITAM 2) and/or SEQ ID NO:47 (CD3zeta ITAM 3); and (b) the second nucleic acid encodes the second polypeptide which comprises a plurality of regions ordered from the amino terminus to the carboxyl terminus: (i) a heavy chain leader sequence comprising the amino acid sequence of SEQ ID NO:54; (ii) a BCMA antibody heavy chain variable region comprising an amino acid sequence having at least 95% sequence identity to the amino acid sequence of any one of SEQ ID NO: 6, 12, 14, 16, 18, 20, 22, 24, 26, or 28; and (iii) a BCMA antibody heavy chain constant region comprising the amino acid sequence of SEQ ID NO:7 or 29. In one embodiment, the first nucleic acid encodes the first polypeptide chain that lacks a light chain leader region and/or lacks a hinge region. In one embodiment, the second nucleic acid encodes the second polypeptide chain that lacks a heavy chain leader region. In one embodiment, the first and second polypeptide chains are exemplified in FIG. 2A or B.

### Host Cells

The present disclosure provides a host cell, or a population of host cells, which harbors one or more expression vectors operably linked to a nucleic acid transgene that encodes any of the first polypeptide chains, second polypeptide chains, first and second polypeptide chains, dimeric antigen receptors or precursor polypeptides described herein.

In one embodiment, the host cell or population of host cells are introduced with one or more expression vectors, where the vectors are operably linked to a nucleic acid transgene encoding any of the dimeric antigen receptor (DAR) constructs described herein. The host cell or the population of host cells comprise T lymphocytes (e.g., T cells, regulatory T cells, gamma-delta T cells, and cytotoxic T cells), NK (natural killer) cells, macrophages, dendritic cells, mast cells, eosinophils, B lymphocytes, monocytes. In one embodiment, the NK cells comprise cord blood-derived NK cells, or placental derived NK cells.

In one embodiment, the host cell or population of host cells are autologous and are derived from the subject to receive treatment (e.g., recipient subject) of host cells expressing dimeric antigen receptors (DAR). In one embodiment, blood (e.g., whole blood) can be obtained from the recipient subject, the desired cells (e.g., T cells) can be recovered/enriched from the recipient subject's blood, and autologous transgenic cells can be prepared by introducing into the desired cells one or more expression vectors operably linked to nucleic acids encoding any of the dimeric antigen receptors described herein. Administering to the recipient subject autologous transgenic T cells expressing a dimeric antigen receptor construct can greatly reduce graft-versus-host disease in the subject.

In one embodiment, the host cell or population of host cells used to treat the subject are allogeneic and are derived from a different subject (e.g., donor subject) or from multiple donor subjects. In one embodiment, the donor subject(s) will not receive treatment of host cells expressing dimeric antigen receptors (DAR). In one embodiment, allogeneic host cells include syngeneic host cells derived from an identical twin donor who will not receive treatment of host cells expressing dimeric antigen receptors (DAR). Allogeneic cells can be obtained from blood (e.g., whole blood) from at least one donor in a similar manner employed for the autologous cells. In one embodiment, blood (e.g., whole blood) can be obtained from at least one donor, the desired cells can be recovered/enriched from the donor's (or donors') blood, and allogeneic transgenic cells can be prepared by introducing into the donor's (or donors') desired cells one or more expression vectors operably linked to nucleic acids encoding any of the dimeric antigen receptors described herein. Administering to the subject allogeneic transgenic T cells expressing a dimeric antigen receptor construct can lead to graft-versus-host disease in the subject.

In one embodiment, the desired cells recovered from the subject's blood, or from the donors' blood, include T lymphocytes (e.g., T cells, regulatory T cells, gamma-delta T cells, and cytotoxic T cells), NK (natural killer) cells, macrophages, dendritic cells, mast cells, eosinophils, B lymphocytes, monocytes. In one embodiment, the NK cells comprise cord blood-derived NK cells, or placental derived NK cells.

In one embodiment, the host cell or population of host cells harbor one or more expression vectors that can direct transient introduction of the transgene into the host cells or stable insertion of the transgene into the host cells' genome, where the transgene comprises nucleic acids encoding any of the dimeric antigen receptors described herein. The expression vector(s) can direct transcription and/or translation of the transgene in the host cell. The expression vectors can include one or more regulatory sequences, such as inducible and/or constitutive promoters and enhancers. The expression vectors can include ribosomal binding sites and/or polyadenylation sites. In one embodiment, the expression vector, which is operably linked to the nucleic acid encoding the dimeric antigen receptor (DAR) construct, can direct production of the dimeric antigen receptor (DAR) construct which can be displayed on the surface of the transgenic host cell or the dimeric antigen receptor can be secreted into the cell culture medium. In one embodiment, host cells can harbor one or more expression vectors operably linked to the nucleic acid transgene that encodes any of the dimeric antigen receptors, and the host cells can be cultured in an appropriate culture medium to transiently or stably express a dimeric antigen receptor construct.

In one embodiment, the host cell or population of host cells harbor one or more expression vectors comprising nucleic acid backbone sequences derived from a virus, for example retrovirus, lentivirus or adenovirus. In one embodiment, the expression vector can include the transgene and sequences for homologous directed repair for use with a CRISPR (cluster regularly interspaced short palindromic repeats) system for insertion or replacement of the transgene into the host cell's genome. In one embodiment, the transgene used in a CRISPR system can be operably joined to a promoter for mediating constitutive or inducible transcription of the dimeric antigen receptor. In one embodiment, CRISPR includes Cas9 or Cpf1 (Cas12a). In one embodiment, the expression vector comprises a transgene in a transposon for use with a transposase-based system. Examples of transposase systems include commercially-available systems such as PIGGYBAC, SUPER PIGGYBAC and SLEEPING BEAUTY (including SB100X).

The present disclosure provides a host cell, or a population of host cells, which harbors an expression vector operably linked to a nucleic acid that encodes a precursor polypeptide comprising: a plurality of regions ordered from the amino terminus to the carboxyl terminus: (i) a heavy chain leader region, (ii) an antibody heavy chain variable region, (iii) an antibody heavy chain constant region, (iv) a hinge region, (v) a transmembrane region, (vi) an intracellular region having two to five intracellular co-stimulatory and/or signaling sequences, (vii) a self-cleaving sequence region, (viii) a light chain leader region, (ix) an antibody light chain variable region, and (x) an antibody light chain constant region. In one embodiment, the nucleic acid encoding the precursor polypeptide lacks a hinge region. In one embodiment, the nucleic acid encoding the precursor polypeptide lacks a heavy chain leader region and/or a light chain leader region. In one embodiment, a precursor polypeptide is exemplified in FIG. 3A or B. In one embodiment, the host cell, or population of host cells, harbors an expression vector operably linked to a nucleic acid encoding any one of the precursor polypeptides having the amino acid sequence of SEQ ID NO:63, 66, 69, 72, 75, 78, 81 or 84. In one embodiment, the host cell, or population of host cells, expresses the precursor polypeptide.

The present disclosure provides a host cell, or a population of host cells, which harbors an expression vector operably linked to a nucleic acid that encodes a precursor polypeptide comprising: a plurality of regions ordered from the amino terminus to the carboxyl terminus: (i) a light chain leader region, (ii) an antibody light chain variable region, (iii) an antibody light chain constant region, (iv) a hinge region, (v) a transmembrane region, (vi) an intracellular region having two to five intracellular co-stimulatory and/or signaling sequences, (vii) a self-cleaving sequence region, (viii) a heavy chain leader region, (ix) an antibody heavy chain variable region, and (x) an antibody heavy chain constant region. In one embodiment, the nucleic acid encoding the precursor polypeptide lacks a hinge region. In one embodiment, the nucleic acid encoding the precursor polypeptide lacks a heavy chain leader region and/or a light chain leader region. In one embodiment, a precursor polypeptide is exemplified in FIG. 4A or B. In one embodiment, the host cell, or population of host cells, expresses the precursor polypeptide.

In one embodiment, the host cell, or the population of host cells, harbors an expression vector operably linked to a nucleic acid that encodes the first and second polypeptide chains, comprising: (a) a first polypeptide chain comprising a plurality of regions ordered from the amino terminus to the carboxyl terminus: (i) a heavy chain leader sequence, (ii) an antibody heavy chain variable region, (iii) an antibody heavy chain constant region, (iv) a hinge region, (v) a transmembrane region, and (vi) an intracellular region having two to five intracellular sequences; and (b) a second polypeptide chain comprising: a plurality of regions ordered from the amino terminus to the carboxyl terminus: (i) an antibody light chain variable region, and (ii) an antibody light chain constant region. In one embodiment, the first nucleic acid encodes a first polypeptide chain that lacks a heavy chain leader region and/or lacks a hinge region. In one embodiment, the first polypeptide chain is exemplified in FIG. 1A or B. In one embodiment, the second nucleic acid encodes a second polypeptide chain that lacks a light chain leader region. In one embodiment, the second polypeptide chain is exemplified in FIG. 1A or B. In one embodiment, the host cell, or the population of host cells, expresses the first and second polypeptide chains.

In one embodiment, the host cell, or the population of host cells, harbors an expression vector operably linked to a nucleic acid that encodes the first and second polypeptide chains, comprising: (a) a first polypeptide chain comprising a plurality of regions ordered from the amino terminus to the carboxyl terminus: (i) a light chain leader sequence, (ii) an antibody light chain variable region, (iii) an antibody light chain constant region, (iv) a hinge region, (v) a transmembrane region, and (vi) an intracellular region having two to five intracellular sequences; and (b) a second polypeptide chain comprising: a plurality of regions ordered from the amino terminus to the carboxyl terminus: (i) an antibody heavy chain variable region, and (ii) an antibody heavy chain constant region. In one embodiment, the first nucleic acid encodes a first polypeptide chain that lacks a heavy chain leader region and/or lacks a hinge region. In one embodiment, the first polypeptide chain is exemplified in FIG. 2A or B. In one embodiment, the second nucleic acid encodes a second polypeptide chain that lacks a light chain leader region. In one embodiment, the second polypeptide chain is exemplified in FIG. 2A or B. In one embodiment, the host cell, or the population of host cells, expresses the first and second polypeptide chains.

In one embodiment, the host cell, or the population of host cells, harbors a first expression vector operably linked to a nucleic acid that encodes the first polypeptide chain and harbors a second expression vector operably linked to a nucleic acid that encodes the second polypeptide chain, wherein (a) the first polypeptide chain comprising a plurality of regions ordered from the amino terminus to the carboxyl terminus: (i) a heavy chain leader sequence, (ii) an antibody heavy chain variable region, (iii) an antibody heavy chain constant region, (iv) a hinge region, (v) a transmembrane region, and (vi) an intracellular region having two to five intracellular sequences; and wherein (b) the second polypeptide chain comprising: a plurality of regions ordered from the amino terminus to the carboxyl terminus: (i) an antibody light chain variable region, and (ii) an antibody light chain constant region. In one embodiment, the first nucleic acid encodes a first polypeptide chain that lacks a heavy chain leader region and/or lacks a hinge region. In one embodiment, the first polypeptide chain is exemplified in FIG. 1A or B. In one embodiment, the second nucleic acid encodes a second polypeptide chain that lacks a light chain leader region. In one embodiment, the second polypeptide chain is exemplified in FIG. 1A or B. In one embodiment, the host cell, or the population of host cells, expresses the first and second polypeptide chains.

In one embodiment, the host cell, or the population of host cells, harbors a first expression vector operably linked to a nucleic acid that encodes the first polypeptide chain and harbors a second expression vector operably linked to a nucleic acid that encodes the second polypeptide chain, wherein (a) the first polypeptide chain comprising a plurality of regions ordered from the amino terminus to the carboxyl terminus: (i) a light chain leader sequence, (ii) an antibody light chain variable region, (iii) an antibody light chain constant region, (iv) a hinge region, (v) a transmembrane region, and (vi) an intracellular region having two to five intracellular sequences; and wherein (b) the second polypeptide chain comprising: a plurality of regions ordered from the amino terminus to the carboxyl terminus: (i) an antibody heavy chain variable region, and (ii) an antibody heavy chain constant region. In one embodiment, the first nucleic acid encodes a first polypeptide chain that lacks a heavy chain leader region and/or lacks a hinge region. In one embodiment, the first polypeptide chain is exemplified in FIG. 2A or B. In one embodiment, the second nucleic acid encodes a second polypeptide chain that lacks a light chain leader region. In one embodiment, the second polypeptide chain is exemplified in FIG. 2A or B. In one embodiment, the host cell, or the population of host cells, expresses the first and second polypeptide chains.

The present disclosure provides a first host cell, or a first population of host cells, which harbors a first expression vector operably linked to a nucleic acid that encodes a first polypeptide chain comprising: a plurality of regions ordered from the amino terminus to the carboxyl terminus: (i) a heavy chain leader region, (ii) an antibody heavy chain variable region, (iii) an antibody heavy chain constant region, (iv) a hinge region, (v) a transmembrane region, and (vi) an intracellular region. In one embodiment, the first nucleic acid encodes a first polypeptide chain that lacks a heavy chain leader region and/or lacks a hinge region. In one embodiment, the first polypeptide chain is exemplified in FIG. 1A or B. In one embodiment, the first host cell, or the first population of host cells, expresses the first polypeptide chains.

The present disclosure provides a second host cell, or a second population of host cells, which harbors a second expression vector operably linked to a nucleic acid that encodes a second polypeptide chain comprising: a plurality of regions ordered from the amino terminus to the carboxyl terminus: (i) a light chain leader region, (ii) an antibody light chain variable region, and (iii) an antibody light chain constant region. In one embodiment, the second nucleic acid encodes a second polypeptide chain that lacks a light chain leader region. In one embodiment, the second polypeptide chain is exemplified in FIG. 1A or B. In one embodiment, the second host cell, or the second population of host cells, expresses the second polypeptide chains.

The present disclosure provides a first host cell, or a first population of host cells, which harbors a first expression vector operably linked to a nucleic acid that encodes a first polypeptide chain comprising: a plurality of regions ordered from the amino terminus to the carboxyl terminus: (i) a light chain leader region, (ii) an antibody light chain variable region, (iii) an antibody light chain constant region, (iv) a hinge region, (v) a transmembrane region, and (vi) an intracellular region. In one embodiment, the first nucleic acid encodes a first polypeptide chain that lacks a heavy chain leader region and/or lacks a hinge region. In one embodiment, the first polypeptide chain is exemplified in FIG. 2A or B. In one embodiment, the first host cell, or the first population of host cells, expresses the first polypeptide chains.

The present disclosure provides a second host cell, or a second population of host cells, which harbors a second expression vector operably linked to a nucleic acid that encodes a second polypeptide chain comprising: a plurality of regions ordered from the amino terminus to the carboxyl terminus: (i) a heavy chain leader region, (ii) an antibody heavy chain variable region, and (iii) an antibody light chain constant region. In one embodiment, the second nucleic acid encodes a second polypeptide chain that lacks a light chain leader region. In one embodiment, the second polypeptide chain is exemplified in FIG. 1A or B. In one embodiment, the second host cell, or the second population of host cells, expresses the second polypeptide chains.

The present disclosure provides a host cell, or a population of host cells, which harbors at least expression vector operably linked to one or more nucleic acids encoding a dimeric antigen receptors, wherein the nucleic acid(s) encode a precursor polypeptide or encode a first and/or second polypeptide chain.

In one embodiment, the BCMA antibody heavy chain variable region comprises the amino acid sequence of any one of SEQ ID NO:6, 12, 14, 16, 18, 20, 22, 24, 26, or 28.

In one embodiment, the BCMA antibody heavy chain constant region comprises the amino acid sequence of SEQ ID NO:7 or 29.

In one embodiment, the hinge region comprises a CD28 hinge region comprising the amino acid sequence of SEQ ID NO:35 and optionally a CD8 hinge region comprising the amino acid sequence of SEQ ID NO:34.

In one embodiment, the transmembrane region comprises a CD28 transmembrane region comprising any one of the amino acid sequence of SEQ ID NO:37 (from CD28), SEQ ID NO:38 (from CD8), SEQ ID NO:39 (from 4-1BB), or SEQ ID NO:40 (from CD3zeta).

In one embodiment, the intracellular region comprises in any order and any combination of two to five intracellular sequences selected from a group consisting of SEQ ID NO:41 (from 4-1BB), SEQ ID NO:42 (from CD28), SEQ ID NO:43 (from OX40), SEQ ID NO:44 (CD3zeta ITAM 1, 2 and 3), SEQ ID NO:45 (CD3zeta ITAM 1), SEQ ID NO:46 (CD3zeta ITAM 2) and/or SEQ ID NO:47 (CD3zeta ITAM 3).

In one embodiment, the BCMA antibody light chain variable region comprises the amino acid sequence of any one of SEQ ID NO:8, 9, 10, 13, 15, 17, 19, 21, 23, 25, 27 or 30.

In one embodiment, the BCMA antibody light chain constant region comprises the amino acid sequence of SEQ ID NO:11 or 31.

In one embodiment, the heavy chain leader sequence comprises the amino acid sequence of SEQ ID NO:54 or 56. In one embodiment, the light chain leader sequence comprises the amino acid sequence of SEQ ID NO:55 or 56.

In one embodiment, the self-cleaving sequence comprises the amino acid sequence of any one of SEQ ID NO:57, 58, 59 or 60.

### Compositions and Pharmaceutical Compositions

The present disclosure provides a composition comprising a population of transgenic host cells that have been engineered to express any one of the dimeric antigen receptor (DAR) constructs, including any one of V1, V2a, V2b, V2c, V3a, V3b, V3c or V4 dimeric antigen receptor (DAR). In one embodiment, the selection of the population of transgenic host cells can be based on the type of disease to be treated and/or the type of response desired in the subject.

In one embodiment, the composition includes a plurality of a transgenic host cell which expresses a dimeric antigen receptor (DAR) that binds a BCMA antigen including any one of V1, V2a, V2b, V2c, V3a, V3b, V3c or V4 dimeric antigen receptor (DAR). In one embodiment, the plurality of the transgenic host cell harbors at least one expression vector operably linked to one or more nucleic acids encoding any of the first polypeptide chains or second polypeptide chains, or any of the first and second polypeptide chains, or any of the precursor polypeptide chains described herein, which can be expressed and processed by the transgenic host cells to generate the first and second polypeptides that associate with each other to form the dimeric antigen receptor (DAR) construct. In one embodiment, the population of transgenic host cells is admixed with a pharmaceutically-acceptable excipient.

The present disclosure provides a composition comprising a combination of two or more populations of transgenic host cells that express different dimeric antigen receptor (DAR) constructs. In one embodiment, the composition comprises a first and a second population of transgenic host cells where the first and second populations have been engineered to express a different dimeric antigen receptor (DAR) construct. In one embodiment, the selection of the first and second population of transgenic host cells can be based on the type of disease to be treated and/or the type of response desired in the subject.

In one embodiment, the composition includes a first population which comprises a plurality of a first transgenic host cell which express a first type of a dimeric antigen receptor (DAR) that binds a BCMA antigen including any one of V1, V2a, V2b, V2c, V3a, V3b, V3c or V4 dimeric antigen receptor (DAR). In one embodiment, the plurality of the first transgenic host cell harbors at least one expression vector operably linked to one or more nucleic acids encoding any of the first polypeptide chains or second polypeptide chains, or any of the first and second polypeptide chains, or any of the precursor polypeptide chains described herein, where the nucleic acid(s) can be expressed by the transgenic host cell and the expressed polypeptide(s) can be processed by the transgenic host cells to generate the first and second polypeptides that associate with each other to form the first type of dimeric antigen receptor (DAR) construct. In one embodiment, the first population of transgenic host cells is admixed with a pharmaceutically-acceptable excipient.

In one embodiment, the composition includes a second population which comprises a plurality of a second transgenic host cell which can express a second type of a dimeric antigen receptor (DAR) that binds a BCMA antigen including a V1, V2a, V2b, V2c, V3a, V3b, V3c or V4 dimeric antigen receptor (DAR), wherein the second type of dimeric antigen receptor (DAR) differs from the first type of dimeric antigen receptor (DAR). In one embodiment, the plurality of the second transgenic host cell harbors at least one expression vector operably linked to one or more nucleic acids encoding any of the first polypeptide chains or second polypeptide chains, or any of the first and second polypeptide chains, or any of the precursor polypeptide chains described herein, where the nucleic acid(s) can be expressed by the transgenic host cell and the expressed polypeptide(s) can be processed by the transgenic host cells to generate the first and second polypeptides that associate with each other to form the second type of dimeric antigen receptor (DAR) construct. In one embodiment, the second population of transgenic host cells is admixed with a pharmaceutically-acceptable excipient.

In one embodiment, the selection of the type of first and second DAR-expressing transgenic host cells for the composition can be based on any characteristics of the DAR T cells, including for example the cell killing capability, the capability to develop memory T cells, in vitro expansion capability, in vivo persistence capability, decreased T cell exhaustion property, and/or cryopreservation property.

In one embodiment, the first population of transgenic host cells can express a V1, V2b or V3b dimeric antigen receptor (DAR) which comprise (i) an intracellular region having a CD28 intracellular sequence and (ii) either CD3zeta ITAM 1, 2 and 3, or ITAM 3 intracellular sequences. In one embodiment, the V1-, V2b- or V3b-expressing transgenic host cells (e.g., the first population of transgenic host cells) can induce a strong and rapid effector response when administered to a subject where the response may be mediated by the CD28 intracellular region of the selected DAR T cells.

In one embodiment, the second population of transgenic host cells can express V2a, V3a or V4 dimeric antigen receptor (DAR) which comprise (i) an intracellular region having a 4-1BB intracellular sequence and (ii) either CD3zeta ITAM 1, 2 and 3, or ITAM 3 intracellular sequences. In one embodiment, the V2a-, V3a- or V4-expressing transgenic host cells (e.g., the second population of transgenic host cells) can induce the development of a longer-lasting memory T cell population when administered to a subject where the properties of the DAR T cells may be mediated by the 4-1BB intracellular region of the selected DAR T cells.

In one embodiment, the first or the second population of transgenic host cells can express a V2c or V3c dimeric antigen receptor (DAR) which comprise (i) an intracellular region having CD28 and 4-1BB intracellular sequences and (ii) either CD3zeta ITAM 1, 2 and 3, or ITAM 3 intracellular sequences. In one embodiment, the V2c- or V3c-expressing transgenic host cells (e.g., the first or second population of transgenic host cells) can induce a combination of a strong and rapid effector response, and development of a longer-lasting memory T cell population, in the subject where the properties of the DAR T cells may be mediated by the CD28 and 4-1BB intracellular regions of the selected DAR T cells.

The present disclosure provides a therapeutic composition comprising a mixture of two or more populations of transgenic host cells, comprising at least a first and a second population of transgenic host cells, wherein (i) the first population comprises a first plurality of transgenic host cells harboring at least one expression vector operably linked to one or more nucleic acids encoding any of the first polypeptide chains or second polypeptide chains, or any of the first and second polypeptide chains, or any of the precursor polypeptide chains described herein, which can be expressed and processed by the plurality of first transgenic host cells to generate the first and second polypeptides that associate with each other to form a first dimeric antigen receptor (DAR) construct, and (ii) the second population comprises a second plurality of transgenic host cells harboring at least one expression vector operably linked to one or more nucleic acids encoding any of the first polypeptide chains or second polypeptide chains, or any of the first and second polypeptide chains, or any of the precursor polypeptide chains described herein, which can be expressed and processed by the plurality of second transgenic host cells to generate the first and second polypeptides that associate with each other to form a second dimeric antigen receptor (DAR) construct that differs from the first dimeric antigen receptor (DAR). In one embodiment, the first plurality of host cells express the first dimeric antigen receptor (DAR) constructs which comprises V1, V2a, V2b, V2c, V3a, V3b, V3c or V4. In one embodiment, the second plurality of host cells express the second dimeric antigen receptor (DAR) constructs which comprises V1, V2a, V2b, V2c, V3a, V3b, V3c or V4, where the second DAR construct differs from the first DAR construct. In one embodiment, the therapeutic composition comprises a first amount of the first population of transgenic host cells, and a second amount of the second population of transgenic host cells, where the first and second amounts are the same or different. In one embodiment, the therapeutic composition further comprises a pharmaceutically-acceptable excipient.

### Methods for Treating

The present disclosure further provides methods for conducting adoptive cell therapy by administering to a subject an effective amount of a population of transgenic host cells that have been engineered to express any one of the anti-BCMA dimeric antigen receptor (DAR) constructs, including any one of V1, V2a, V2b, V2c, V3a, V3b, V3c or V4 DAR constructs. The selection of the DAR-expressing transgenic host cells can be based on the type of disease to be treated and the type of response desired in the subject.

The present disclosure further provides a method of treating a subject having a disease, disorder or condition associated with detrimental expression (e.g., elevated expression) of a tumor antigen. Such a method comprises administering to the subject an effective amount of a population of host cells which harbor at least one expression vector operably linked to one or more nucleic acids encoding any of the first polypeptide chains or second polypeptide chains, or any of the first and second polypeptide chains, or any of the precursor polypeptide chains described herein. In one embodiment, the host cell or the population of host cells express any of the first and second polypeptide chains, or any of the precursor polypeptide chains described herein.

The present disclosure provides methods for conducting adoptive cell therapy by administering to a subject an effective amount of a combination of at least two populations of transgenic host cells where each population has been engineered to express different dimeric antigen receptor (DAR) constructs. In one embodiment, methods for conducting adoptive cell therapy comprise administering to a subject an effective amount of a combination of a first and a second population of transgenic host cells where the first and second populations have been engineered to express a different dimeric antigen receptor (DAR) construct. The selection of the first and second population of transgenic host cells can be based on the type of disease to be treated and/or the type of response desired in the subject.

In one embodiment, the first population comprises a plurality of a first transgenic host cell which express a first type of a dimeric antigen receptor (DAR) that binds a BCMA antigen including any one of V1, V2a, V2b, V2c, V3a, V3b, V3c or V4 dimeric antigen receptor (DAR). In one embodiment, the plurality of the first transgenic host cell harbors at least one expression vector operably linked to one or more nucleic acids encoding any of the first polypeptide chains or second polypeptide chains, or any of the first and second polypeptide chains, or any of the precursor polypeptide chains described herein, where the nucleic acid(s) can be expressed by the transgenic host cell and the expressed polypeptide(s) processed by the transgenic host cells to generate the first and second polypeptides that associate with each other to form the first type of dimeric antigen receptor (DAR) construct. In one embodiment, the first population of transgenic host cells is admixed with a pharmaceutically-acceptable excipient.

In one embodiment, the second population comprises a plurality of a second transgenic host cell which can express a second type of a dimeric antigen receptor (DAR) that binds a BCMA antigen including a V1, V2a, V2b, V2c, V3a, V3b, V3c or V4 dimeric antigen receptor (DAR), wherein the second type of dimeric antigen receptor (DAR) differs from the first type of dimeric antigen receptor (DAR). In one embodiment, the plurality of the second transgenic host cell harbors at least one expression vector operably linked to one or more nucleic acids encoding any of the first polypeptide chains or second polypeptide chains, or any of the first and second polypeptide chains, or any of the precursor polypeptide chains described herein, where the nucleic acid(s) can be expressed by the transgenic host cell and the expressed polypeptide(s) processed by the transgenic host cells to generate the first and second polypeptides that associate with each other to form the second type of dimeric antigen receptor (DAR) construct. In one embodiment, the second population of transgenic host cells is admixed with a pharmaceutically-acceptable excipient.

In one embodiment, the selection of the type of first and second DAR-expressing transgenic host cells for administering to the subject can be based on any characteristics of the DAR T cells, including for example the cell killing capability, the capability to develop memory T cells, in vitro expansion capability, in vivo persistence capability, decreased T cell exhaustion property, and/or cryopreservation property.

In one embodiment, the first population of transgenic host cells can express a V1, V2b or V3b dimeric antigen receptor (DAR) which comprise (i) an intracellular region having a CD28 intracellular sequence and (ii) either CD3zeta ITAM 1, 2 and 3, or ITAM 3 intracellular sequences. In one embodiment, the V1-, V2b- or V3b-expressing transgenic host cells (e.g., the first population of transgenic host cells) can induce a strong and rapid effector response in the subject which may be mediated by the CD28 intracellular region of the selected DAR T cells.

In one embodiment, the second population of transgenic host cells can express V2a, V3a or V4 dimeric antigen receptor (DAR) which comprise (i) an intracellular region having a 4-1BB intracellular sequence and (ii) either CD3zeta ITAM 1, 2 and 3, or ITAM 3 intracellular sequences. In one embodiment, the V2a-, V3a- or V4-expressing transgenic host cells (e.g., the second population of transgenic host cells) can induce the development of a longer-lasting memory T cell population in the subject which may be mediated by the 4-1BB intracellular region of the selected DAR T cells.

In one embodiment, the first or the second population of transgenic host cells can express a V2c or V3c dimeric antigen receptor (DAR) which comprise (i) an intracellular region having CD28 and 4-1BB intracellular sequences and (ii) either CD3zeta ITAM 1, 2 and 3, or ITAM 3 intracellular sequences. In one embodiment, the V2c- or V3c-expressing transgenic host cells (e.g., the first or second population of transgenic host cells) can induce a combination of a strong and rapid effector response, and development of a longer-lasting memory T cell population, in the subject which may be mediated by the CD28 and 4-1BB intracellular regions of the selected DAR T cells.

In one embodiment, the first and second population of transgenic host cells can be administered to the subject at the same time (e.g., simultaneously or essentially simultaneously).

In one embodiment, the first and second population of transgenic host cells can be administered to the subject sequentially in either order.

In one embodiment, the same dose or different doses of the first and second population of the transgenic host cells can be administered to the subject.

In one embodiment, a single dose of the first and second population of the transgenic host cells can be administered to the subject.

In one embodiment, at least two doses of the first and second population of the transgenic host cells can be administered to the subject.

In one embodiment, the number of doses of the first and second population of the transgenic host cells that are administered to the subject can be the same or different.

The present disclosure provides a method of treating a subject having a disease, disorder or condition associated with detrimental expression of a tumor antigen, wherein the disorder is cancer, including, but not limited to hematologic breast cancer, ovarian cancer, prostate cancer, head and neck cancer, lung cancer, bladder cancer, melanoma, colorectal cancer, pancreatic cancer, lung cancer, liver cancer, renal cancer, esophageal cancer, leiomyoma, leiomyosarcoma, glioma, and glioblastoma.

In one embodiment, the cancer is a hematologic cancer selected from the group consisting of non-Hodgkin's lymphoma (NHL), Burkitt's lymphoma (BL), B chronic lymphocytic leukemia (B-CLL), B and T acute lymphocytic leukemia (ALL), T cell lymphoma (TCL), acute myeloid leukemia (AML), hairy cell leukemia (HCL), Hodgkin's Lymphoma (HL), chronic myeloid leukemia (CML) and multiple myeloma (MM). In some embodiments, the cancer is a BCMA-positive cancer, such as a BCMA-positive hematologic cancer, e.g., a BCMA-positive B-cell hematologic cancer (e.g., lymphoma (such as NHL), leukemia (such as CLL), or myeloma.

### EXAMPLES

The following examples are meant to be illustrative and can be used to further understand embodiments of the present disclosure and should not be construed as limiting the scope of the present teachings in any way.

### Example 1: Isolation of Human PBMC Cells and Primary T Cells

Primary human T cells were isolated from healthy human donors either from buffy coats (San Diego blood bank), fresh blood or leukapheresis products (StemCell). Peripheral blood mononuclear cells (PBMC) were isolated by density gradient centrifugation.

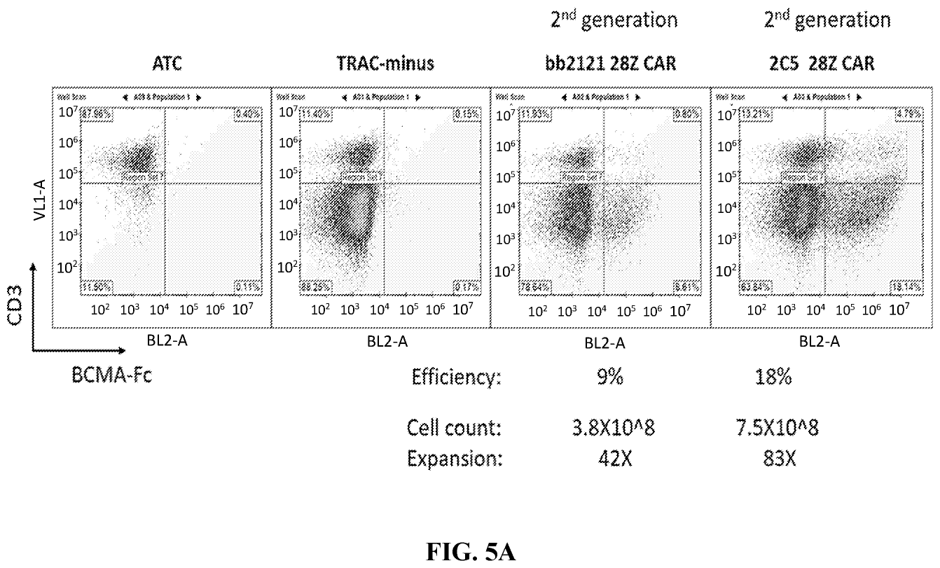

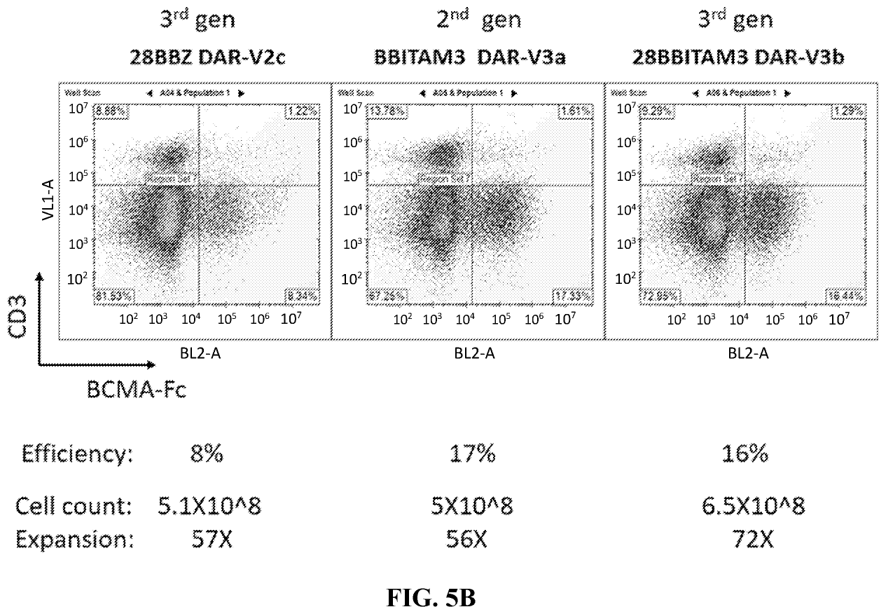

Preparation of Donor 1 cells: T cells were isolated from PBMCs by magnetic negative selection using EASYSEP Human T Cell Isolation Kit (from STEMCELL Technologies, catalog No. 17951) or positive selection and activation by DYNABEADS Human T-Expander CD3/CD28 (from Thermo Fisher Scientific, catalog No. 11141D) according to manufacturer's instructions. Donor 1 cells were introduced with nucleic acids encoding a BCMA CAR or DAR to generate transgenic T cells that express the CAR or DAR constructs. The transgenic cells used for assays to generate data presented in FIGS. 5A-11.

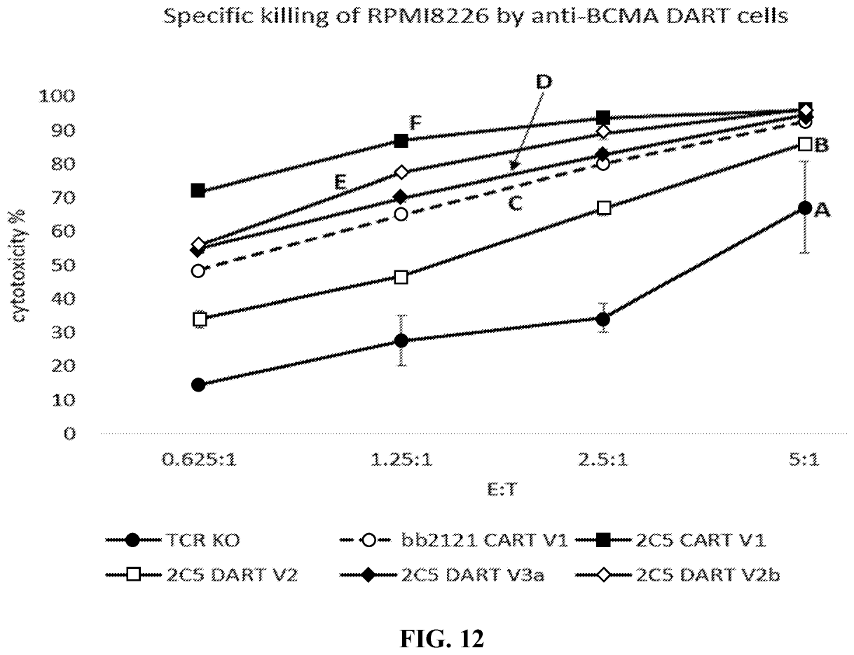

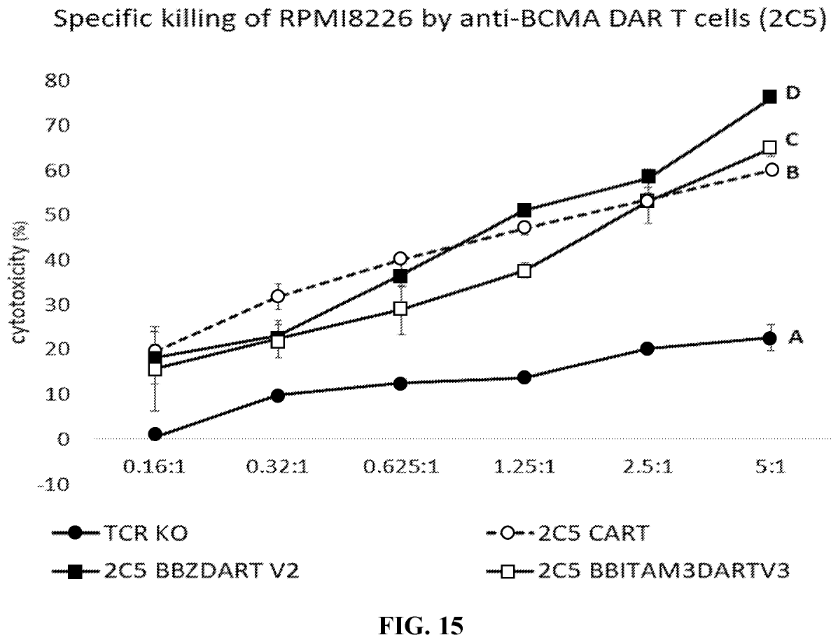

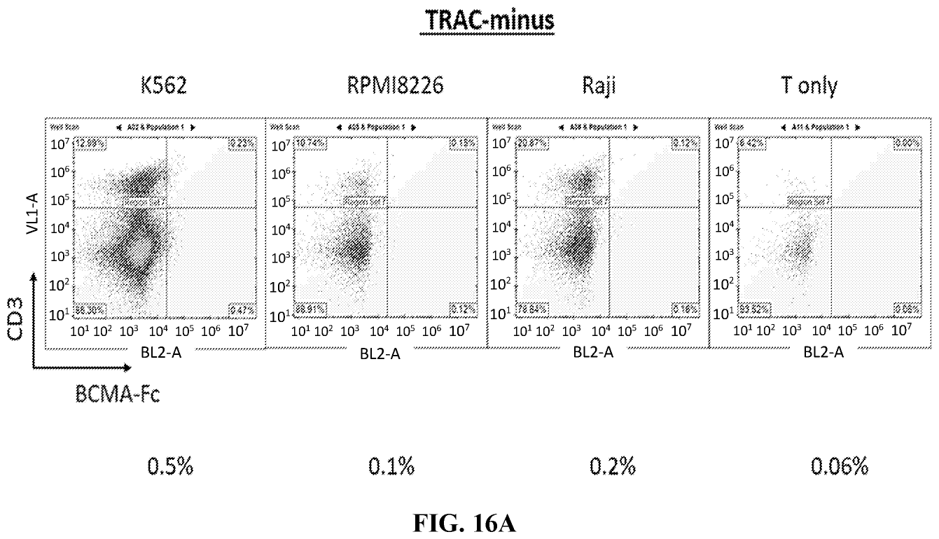

Preparation of Donor 2 cells: To deplete the monocytes, PBMC were plated in the cell culture coated flask for one to two hours. The nonadherent lymphocytes were washed away from the flask and activated with T cell TRANSACT (from Miltenyi, catalog No. 130-111-160) in a new flask according to manufacturer's instructions. Donor 2 cells were introduced with nucleic acids encoding a BCMA CAR or DAR to generate transgenic T cells that express the CAR or DAR constructs. The transgenic cells used for assays to generate data presented in FIGS. 12-15.

### Example 2: Primary T Cell Culture

Primary T cells were cultured in CTS OPTMIZER T Cell Expansion SFM supplemented with 5% CTS Immune Cell SR (Thermo Fisher Scientific) with 300U/mL IL-2 (Proleukin) at a density of 10⁶ cells per mL. Isolated T cells were stimulated freshly or from the frozen tank. Cells were activated with T Cell TRANSACT (Miltenyi) 3 uL/10⁶ cells per mL for two to three days. Following transfection, T cells were cultured in media with IL-2 at 300 U/mL.

### Example 3: Preparation of CAR and DAR T Cells

Activated T cells (approximately 9×10⁶ cells) were introduced with nucleic acids encoding either a CAR construct or a precursor DAR. The naming designation of the BCMA CAR and BCMA DAR constructs, with their respective hinge and intracellular regions is listed in Table 1 below.

TABLE 1

| | | Intracellular | |
|---|---|---|---|
| | | co-stimulatory | Intracellular |
| Designation: | Hinge region: | region: | Signaling region: |
| CAR 28Z | Long | CD28 | CD3zeta (ITAM |
| | CD8 + CD28 | | 1, 2, 3) |
| DAR V1 | Long | CD28 | CD3zeta (ITAM |
| | CD8 + CD28 | | 1, 2, 3) |
| DAR V2a | Short CD28 | 4-1BB | CD3zeta (ITAM |
| | | | 1, 2, 3) |
| DAR V2b | Short CD28 | CD28 | CD3zeta (ITAM |
| | | | 1, 2, 3) |
| DAR V2c | Short CD28 | CD28 and | CD3zeta (ITAM |
| | | 4-1BB | 1, 2, 3) |
| DAR V3a | Short CD28 | 4-1BB | CD3zeta (ITAM 3) |
| DAR V3b | Short CD28 | CD28 and | CD3zeta (ITAM 3) |
| | | 4-1BB | |
| DAR V3c | Short CD28 | CD28 | CD3zeta (ITAM3) |
| DAR V4 | No hinge | 4-1BB | CD3zeta (ITAM 3) |
| DAR 2^{nd} gen | Short CD28 | CD28 or | CD3zeta (ITAM |
| | | 4-1BB | 1, 2, 3) |
| DAR 3^{rd} gen | Short CD28 | CD28 and | CD3zeta (ITAM |
| | | 4-1BB | 1, 2, 3) |

The transgenic CAR and DAR T cells were used fresh or were cryopreserved for future use. For cryopreserved CAR and DAR T cells, the cells were re-suspended in freezing medium (70% AIM-V medium, 20% FBS and 10% DMSO), transferred to a sterile centrifuge tube, and centrifuged at 1300 RPM at 4° C. for 5 minutes. The supernatant was removed and discarded. The cell pellet was quickly re-suspended by adding 2 mL freezing medium to 1×10⁸ cells. The cells were frozen overnight at −80° C. The cells were transferred to −150° C., typically within 1-2 months.

To thaw the cells, the cells were removed from the −150° C. freezer and held in a water bath at 37° C. until thawed. The thawed cells were transferred to a sterile centrifuge tube with 50 mL DPBS, and centrifuged at 1300 RPM for 5 minutes. The supernatant was removed and discarded. Fresh DPBS was added to re-suspend the cells. The cells were counted. The cell concentration was adjusted as needed and filtered through a 30 um cell strainer. The cells were placed on ice until use. The CAR and DAR T cells have been stored at −80° C. for up to two months, or at −150° C. for up to four to six months, and still exhibited excellent in vitro and in vivo tumor killing capability.

### Example 4: Tumor Cell Lines

Multiple myeloma cell line RPMI 8226 was obtained from ATCC and were transduced using a lentivirus carrying luciferase and GFP genes. A single cell clone with luciferase and GFP expression was selected (RPMI8226-FLuc). K562/RPE cells were made similarly by transducing the K562 cells with lentivirus carrying RPE genes. Both cell lines were cultured in RPMI1640 medium (ATCC) supplemented with 10% fetal bovine serum (Sigma).

### Example 5: Transfection Efficiency and Expression Levels of DAR-Expressing T Cells

Transfection and expression levels of either anti-BCMA chimeric antigen receptor (CAR) or anti-BCMA dimeric antigen receptor (DAR) from transgenic T cells were compared using flow cytometry.

The transfection efficiency of transgenic T cells (Donor 1) expressing various BCMA (bb2121 or 2C5) CAR or DAR constructs are similar to each other (FIGS. 5A and B at day 11). The cell expansion level of cells expressing BCMA-2C5 CAR (83X) was nearly twice that of BCMA-bb2121 CAR (42X). The cell expansion level of cells expressing BCMA-2C5 DAR V3b (72X) was higher compared to cells expressing BCMA-2C5 DAR V2c (57X) and V3a (56X) (FIG. 5B). The transfection efficiency of transgenic T cells expressing BCMA bb2121 DAR was less than 10% (data not shown).

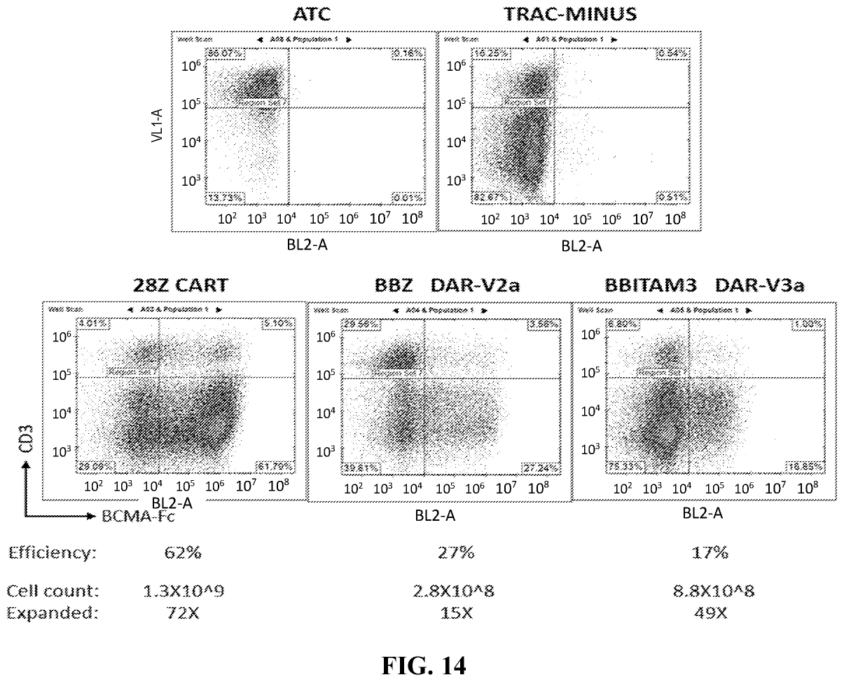

The transfection efficiency of transgenic T cells (Donor 2) expressing BCMA-2C5 CAR or DAR constructs varied, where T cells expressing BCMA-2C5 CAR exhibited higher efficiency (62%) compared to T cells expressing BCMA-2C5 V2a (27%) or V3a (17%). The cell expansion level of cells expressing BCMA-2C5 CAR (72X) was higher compared to cells expressing BCMA-2C5 DAR V2c (15X) or V3a (49X) (FIG. 14).

### Example 6: In Vitro Cytotoxicity Assays

Two to three weeks after preparing the CAR, DAR and control T cells, cells were subjected to nutrient starvation overnight with IL-2. The cells were co-cultured with the target cell mixture of BCMA positive RPMI-8226/GFP cells or BCMA negative K562/RPE cells. The ratio of effector to target cell ranged from 0.16:1 to 5:1. After overnight incubation, the cells were subjected to flow cytometry to measure the GFP cell population to determine the specific target cell killing by anti-BCMA CAR or DAR T cells.

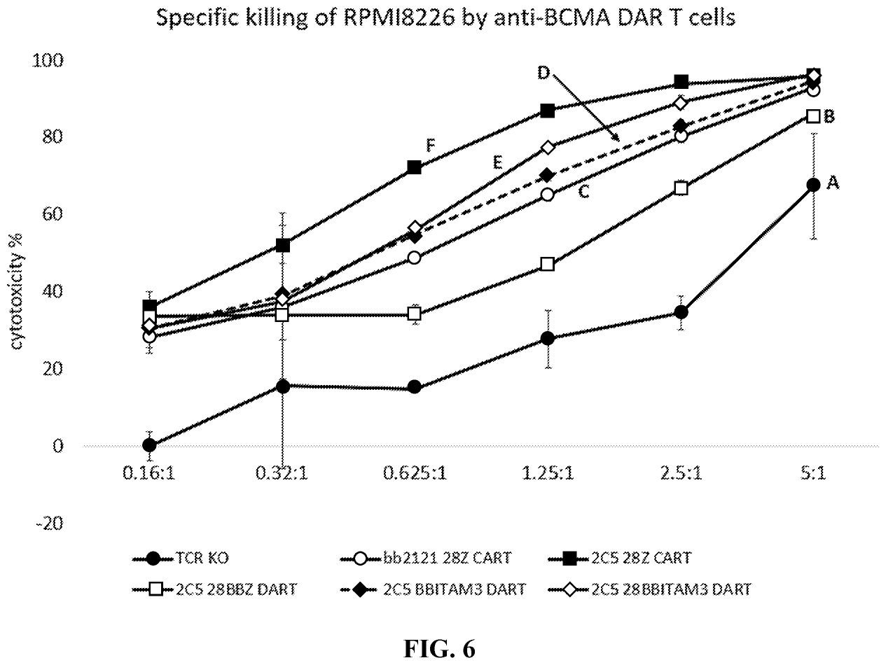

Transgenic cells (Donor 1) expressing BCMA-2C5 CAR (Line F) exhibited a higher level of cell killing compared to BCMA-2C5 DAR V3b (Line E), DAR V3a (Line D) and DAR V3a (Line B). Transgenic cells expressing BCMA bb2121 CAR (Line C) exhibited a higher level of cell killing compared to BCMA-2C5 DAR V3a (Line B) (FIG. 6).

Transgenic cells (Donor 2) expressing BCMA-2C5 DAR V2a (Line D) exhibited a higher level of cell killing compared to cells expressing BCMA-2C5 DAR V3a (Line C) or CAR (Line B) (FIG. 12).

### Example 7: In Vitro Cytokine Secretion Assays

Two to three weeks after preparing the CAR, DAR and control T cells, the cells were subjected to nutrient starvation overnight with IL-2. The cells were co-cultured with BCMA-negative K562, or BCMA-positive U266 or RPMI8226 cells. The ratio of the effector to target cell was 2:1. After 40 hours incubation, the cells were centrifuged to collect the supernatant for detecting cytokine IFN-gamma (ELISA MAX Delux Set, from BioLegend, catalog No. 430104) or GM-CSF (Human Gm-CSF Uncoated ELISA kit from Invitrogen/Thermo Fisher, catalog No. 88-8337) according to the manufacturer's instructions.

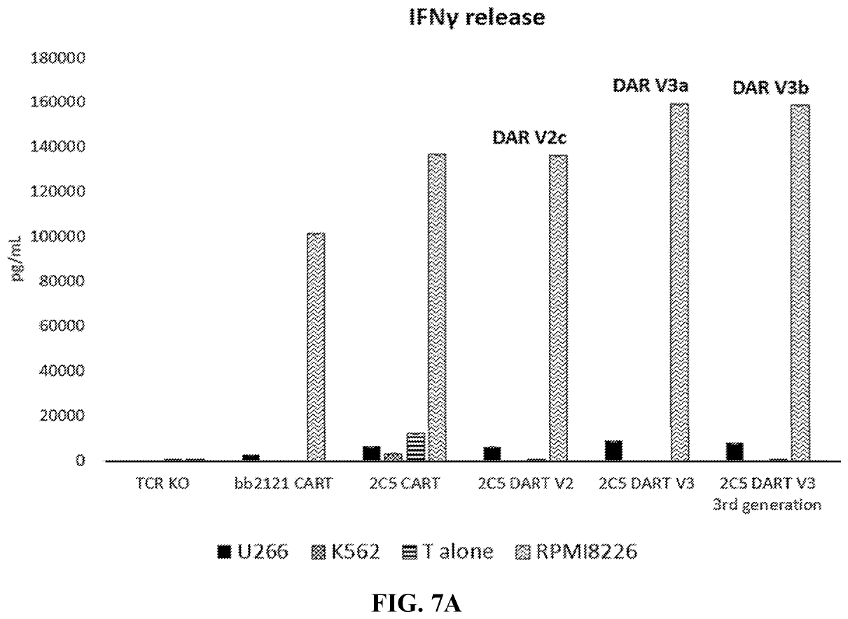

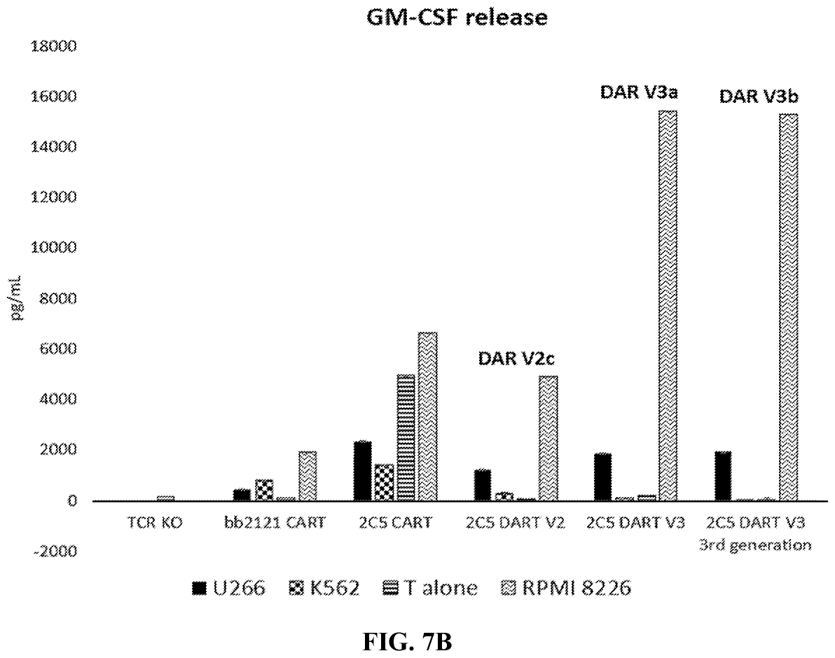

The results in FIG. 7A indicate that T cells (Donor 1) expressing BCMA-2C5 DAR V3a or V3b secrete higher levels of IFN-gamma when co-cultured with RPMI8226 cells compared to BCMA-bb2121 CAR, BCMA-2C5 CAR, or BCMA-2C5 DAR V2c.

The results in FIG. 7B indicate that T cells (Donor 1) expressing BCMA-2C5 DAR V3a or V3b secrete much higher levels of GM-CSF when co-cultured with RPMI8226 cells compared to BCMA-bb2121 CAR, BCMA-2C5 CAR, or BCMA-2C5 DAR V2c.

### Example 8: In Vitro Expansion of Co-Cultured Transgenic Cells

Two to three weeks after preparing the CAR, DAR and control T cells, the cells were subjected to nutrient starvation overnight with IL-2. The cells were co-cultured with BCMA-negative K562, or BCMA-positive U266 or RPMI8226 cells. The level of cell expansion was measured using flow cytometry.

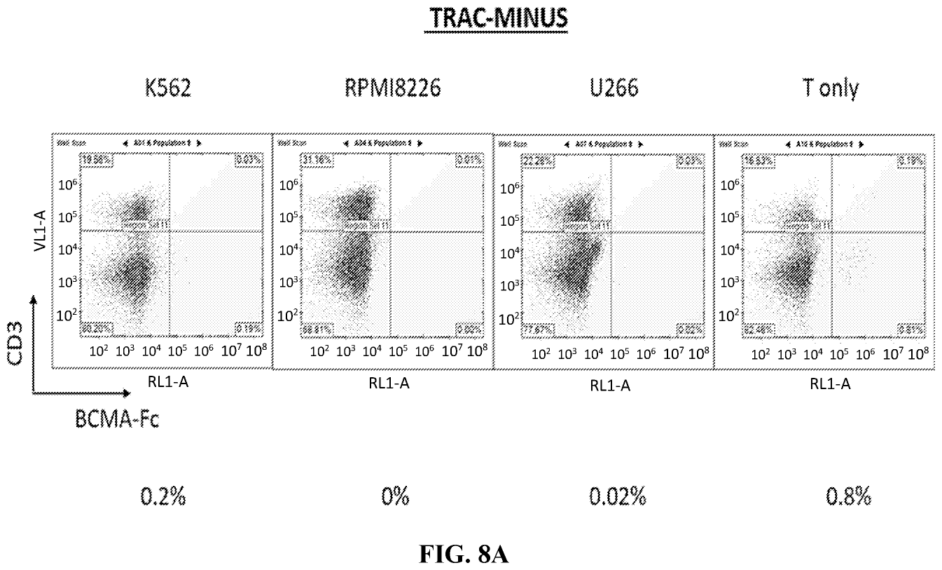

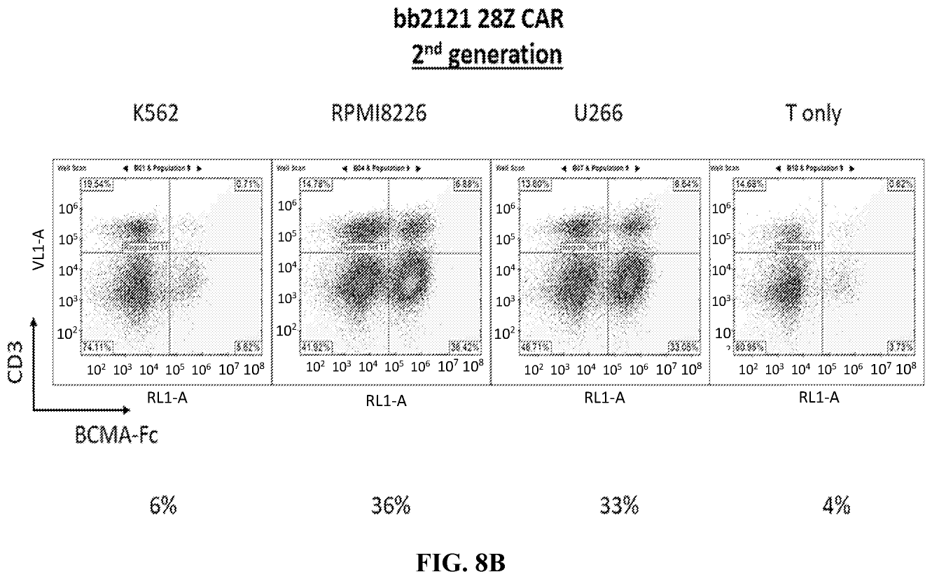

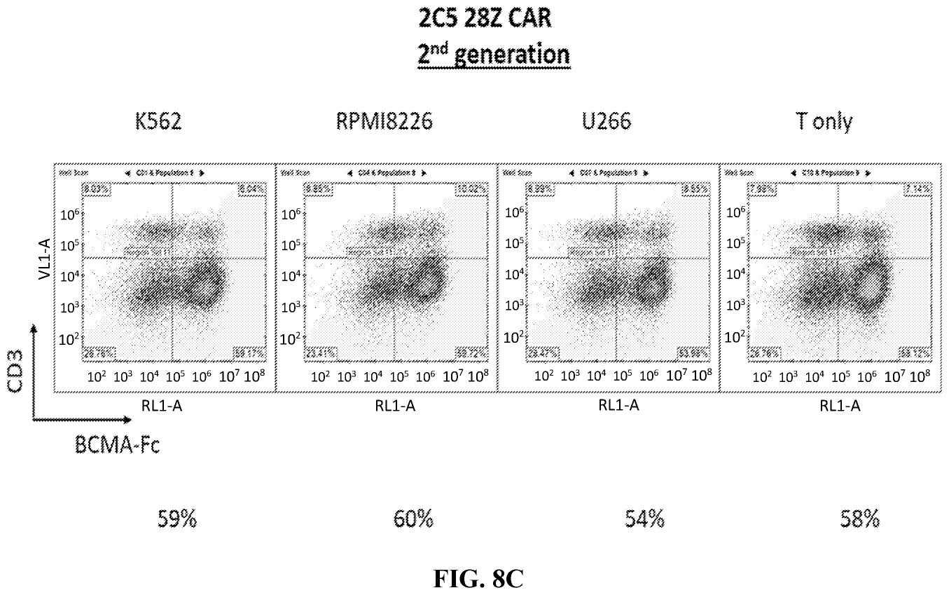

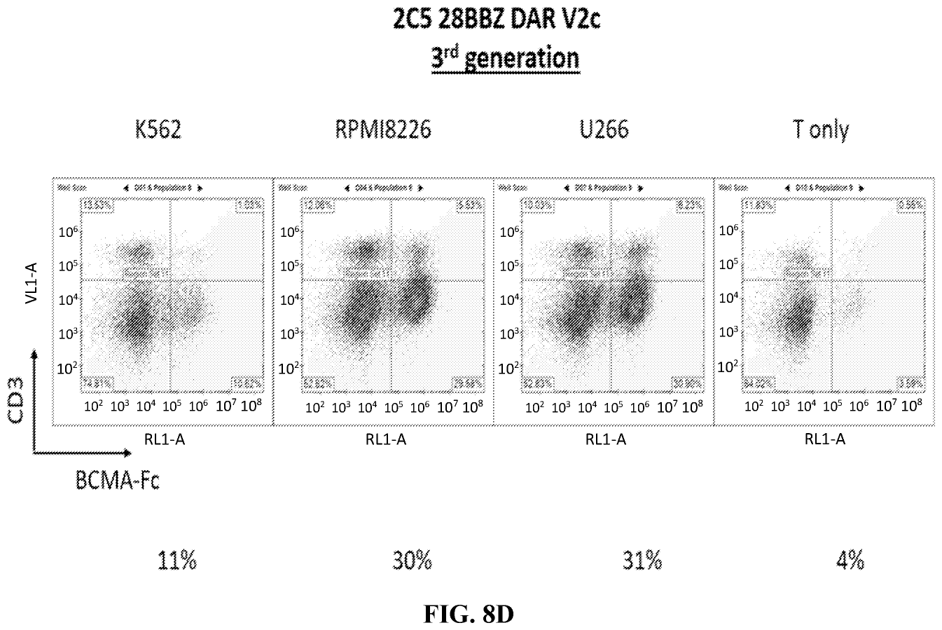

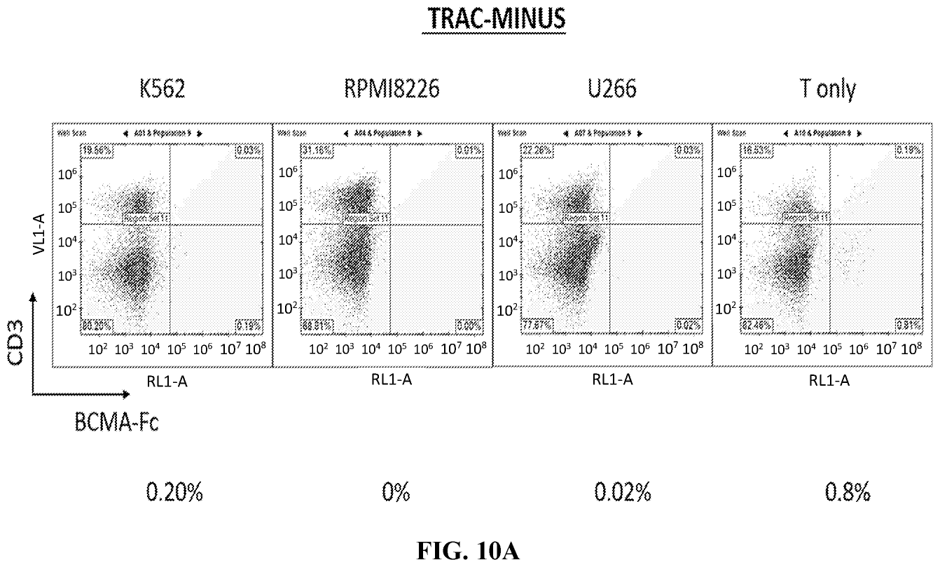

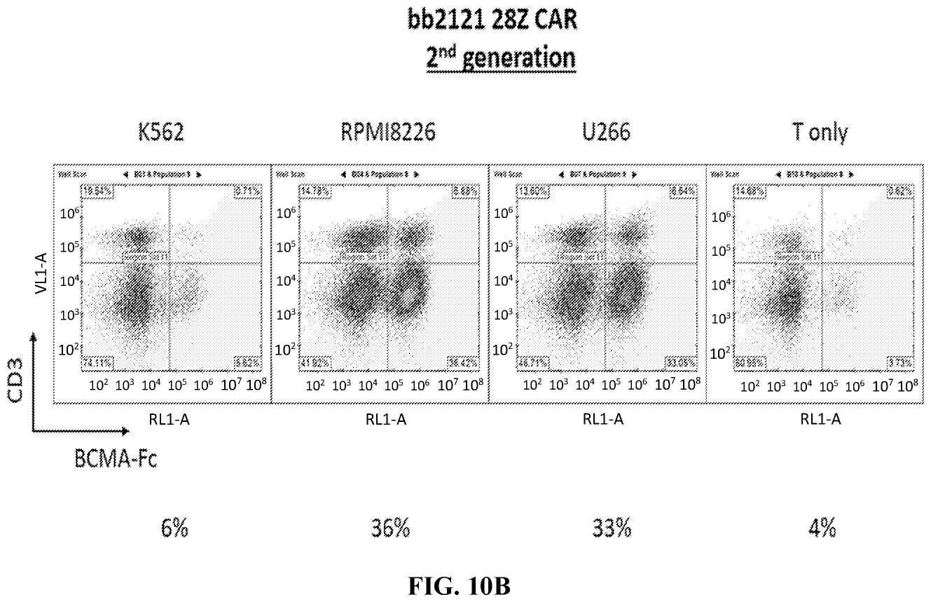

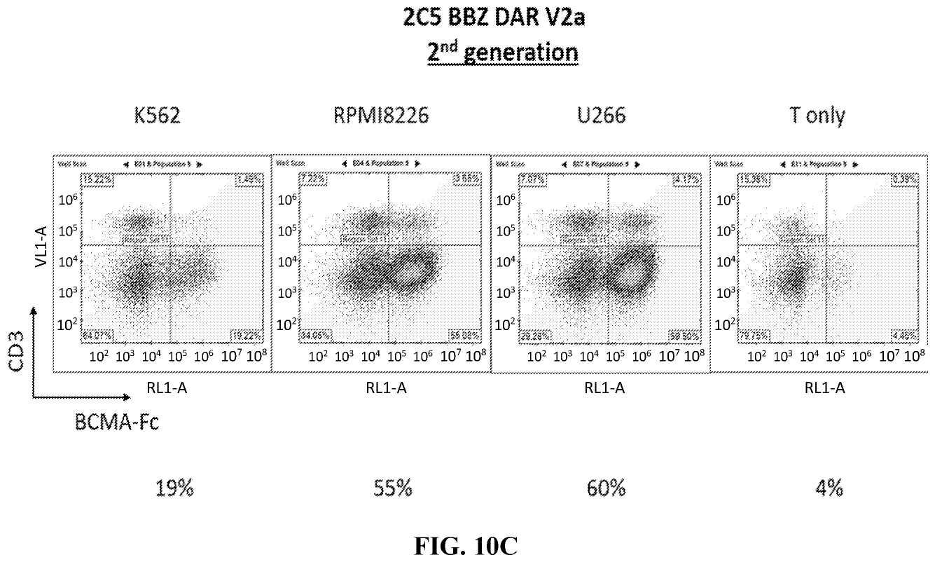

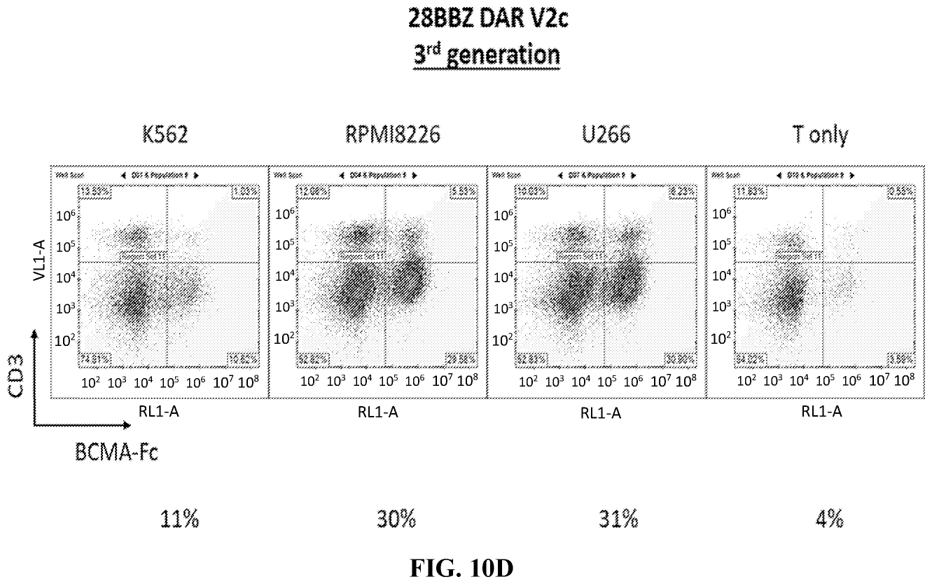

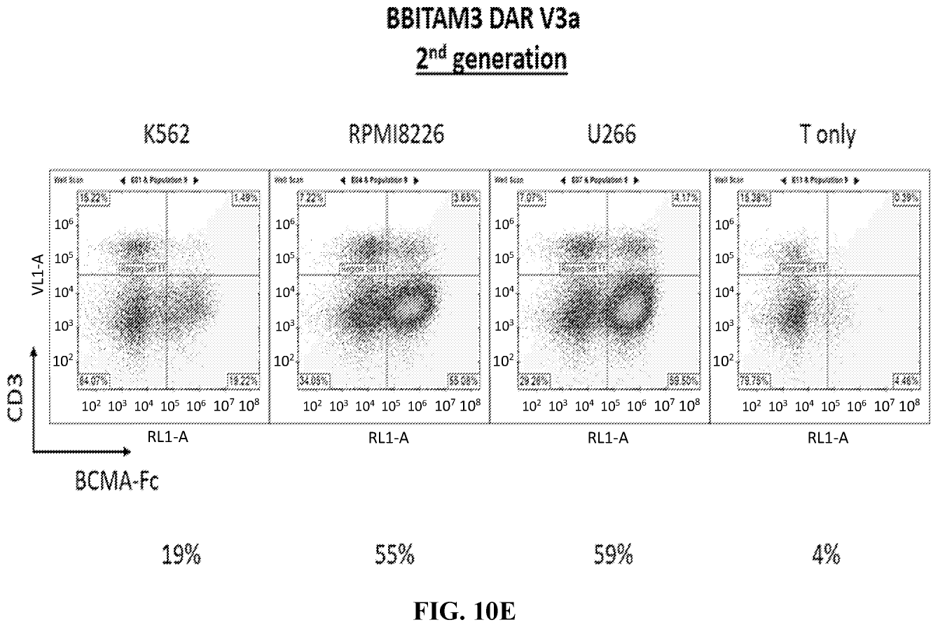

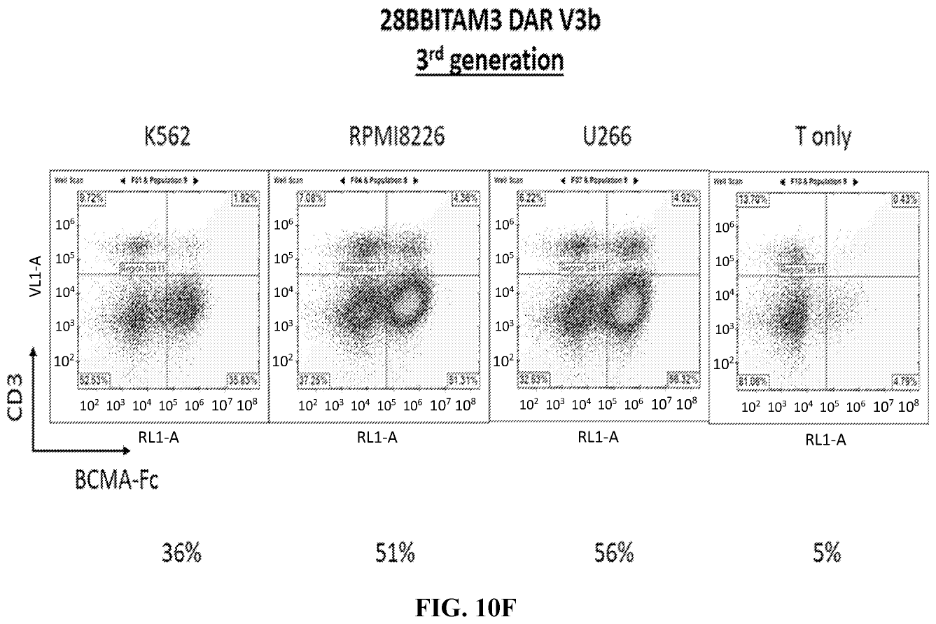

The negative control cells showed little or no expansion (FIGS. 8A and 10A). The transgenic cells (Donor 1) expressing BCMA bb2121 CAR showed higher expansion levels when co-cultured with RPMI8226 or U266 cells compared to co-culture with K562 cells (FIGS. 8B and 10B). The transgenic cells expressing BCMA-2C5 CAR unexpectedly showed high levels of expansion when co-cultured with RPMI8226, U266 and K562 cells (FIG. 8C) indicating non-specific response. The transgenic cells expressing BCMA-2C5 DAR V2c showed higher expansion levels when co-cultured with RPMI8226 or U266 cells compared to co-culture with K562 cells (FIG. 8D).

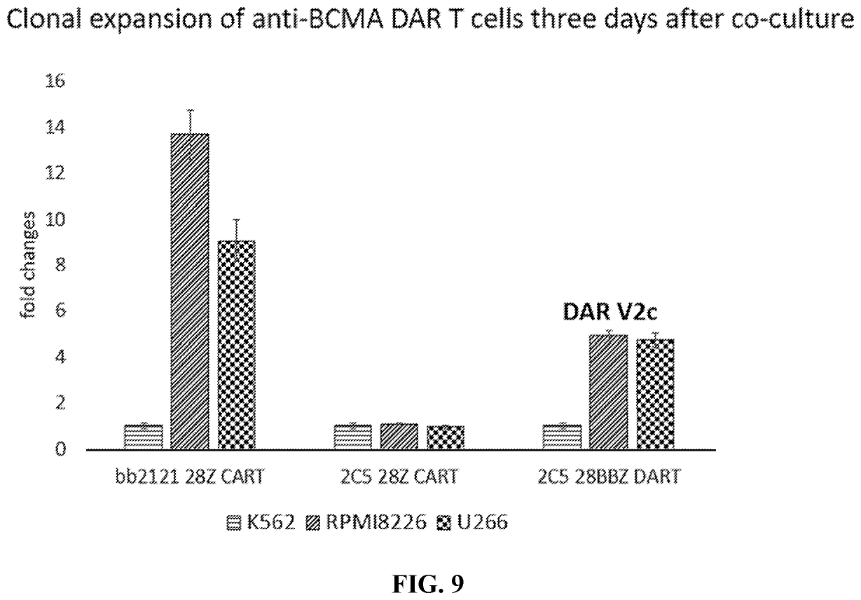

The fold-change in cell expansion of the co-cultured transgenic cells from FIGS. 8A-D is shown in the bar graph of FIG. 9. The transgenic cells (Donor 1) expressing BCMA bb2121 CAR have a higher fold-change in cell expansion when co-cultured with RPMI8226 or U266 cells, compared to transgenic cells expressing BCMA-2C5 DAR V2a. The transgenic cells expressing BCMA-2C5 CAR have a very low fold-change in cell expansion.

The transgenic cells (Donor 1) expressing BCMA-2C5 DAR V2c showed higher expansion levels when co-cultured with RPMI8226 or U266 cells compared to co-culture with K562 cells (FIG. 10D). The transgenic cells expressing BCMA-2C5 DAR V2a (FIG. 10C) or BCMA-2C5 DAR V3a (FIG. 10E) or BCMA-2C5 DAR V3b (FIG. 10F) showed even higher expansion levels when co-cultured with RPMI8226 or U266 cells compared to co-culture with K562 cells.

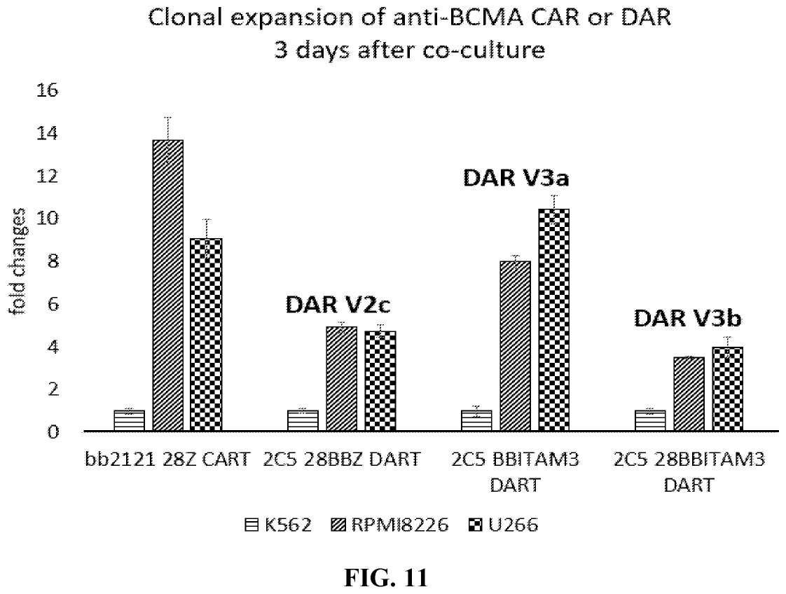

The fold-change in cell expansion of the co-cultured transgenic cells from FIGS. 10B and 10D-10F is shown in the bar graph of FIG. 11. The transgenic cells (Donor 1) expressing BCMA bb2121 CAR have a higher fold-change in cell expansion when co-cultured with RPMI8226 cells, compared to transgenic cells expressing BCMA-2C5 DAR V2c, V3a and V3b. The transgenic cells expressing BCMA-2C5 DAR V3a have a higher fold-change in cell expansion when co-cultured with U266 cells, compared to transgenic cells expressing BCMA bb2121 CAR, or BCMA-2C5 DAR V2c or DAR V3b.

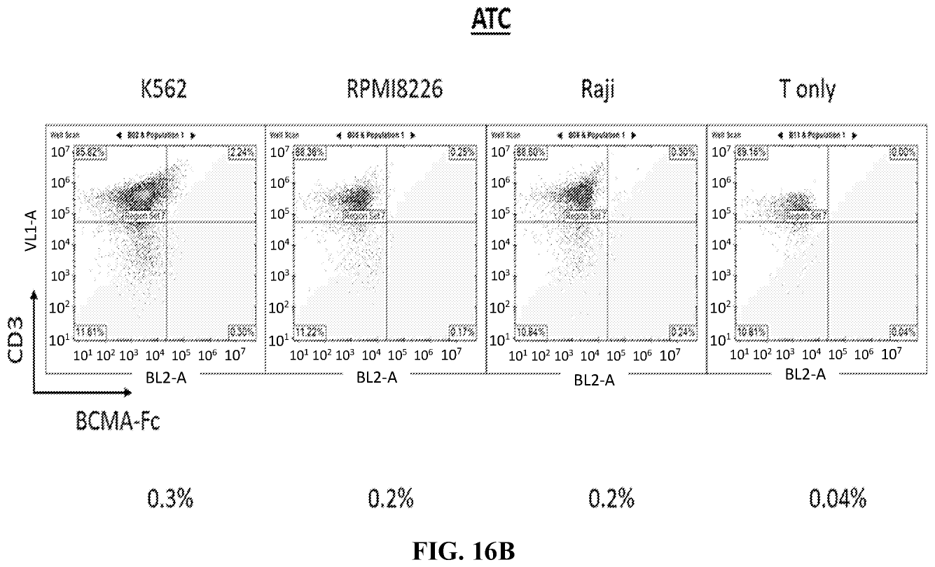

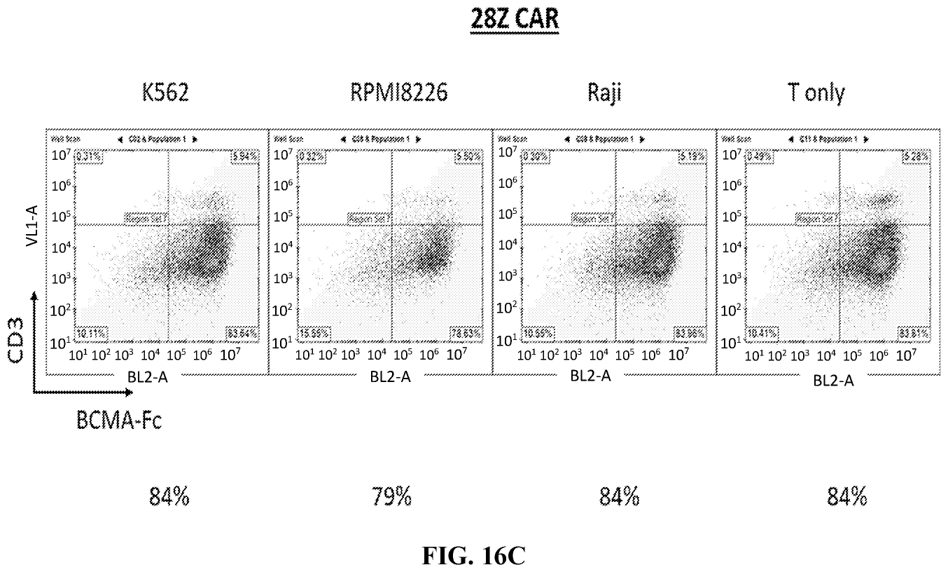

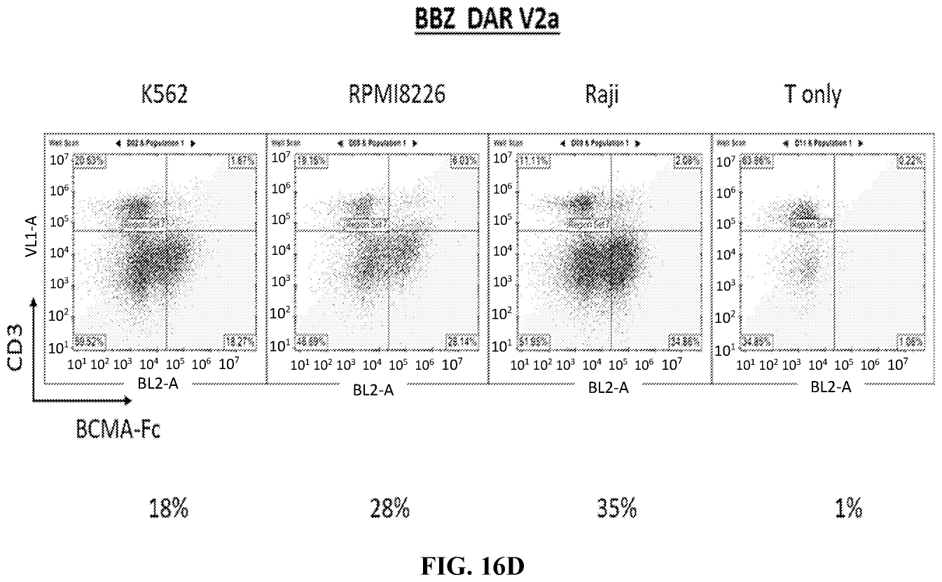

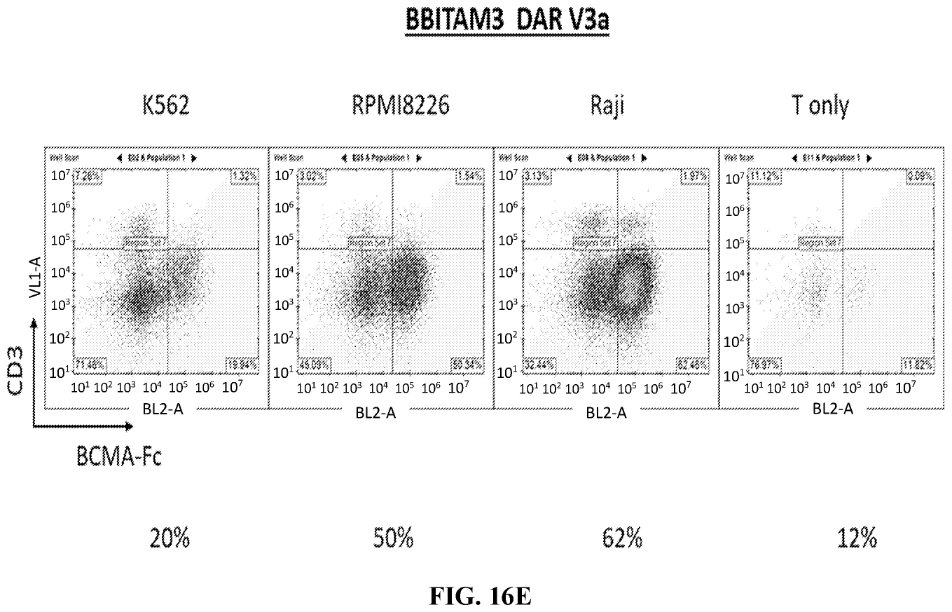

The negative control cells (TCR-minus and ATC) showed little or no expansion when co-cultured with K562, RPMI8226 or Raji cells (FIGS. 16A and B). The transgenic cells (Donor 2) expressing BCMA-2C5 CAR unexpectedly showed high levels of expansion when co-cultured with K562, RPMI8226 or Raji cells (FIG. 16C) indicating non-specific response. The transgenic cells expressing BCMA-2C5 DAR V2a showed higher expansion levels when co-cultured with Raji cells compared to co-culture with K562 or RPMI8226 cells (FIG. 16D). The transgenic cells expressing BCMA-2C5 DAR V3a showed higher expansion levels when co-cultured with Raji cells compared to co-culture with K562 or RPMI8226 cells (FIG. 16E).

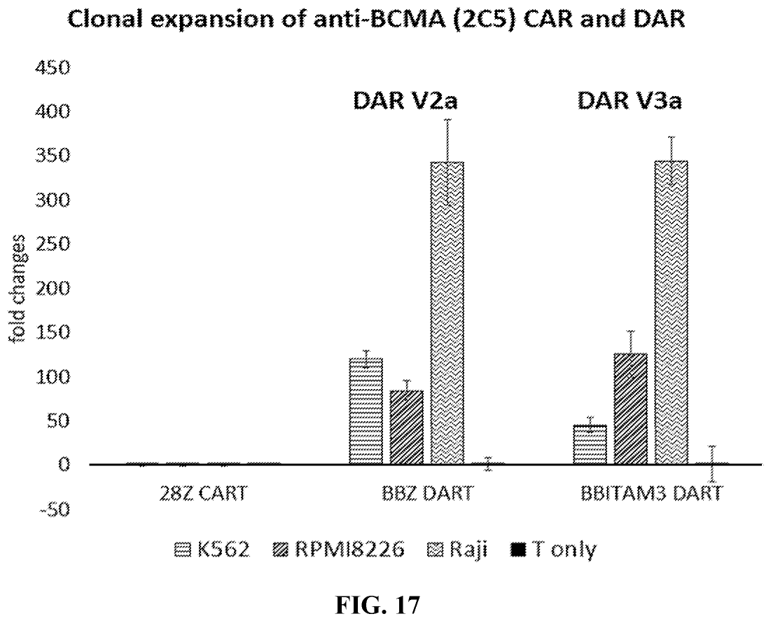

The fold-change in cell expansion of the co-cultured transgenic cells from FIGS. 16C-E is shown in the bar graph of FIG. 17. The transgenic cells (Donor 2) expressing BCMA-2C5 DAR V2a and V3a have a similar higher fold-change in cell expansion when co-cultured with Raji cells, compared to transgenic cells expressing BCMA-2C5 DAR V2a and V3a when co-cultured with K562 or RPMI8226 cells.

### Example 9: Detecting Memory T Cells and Central Memory T Cells

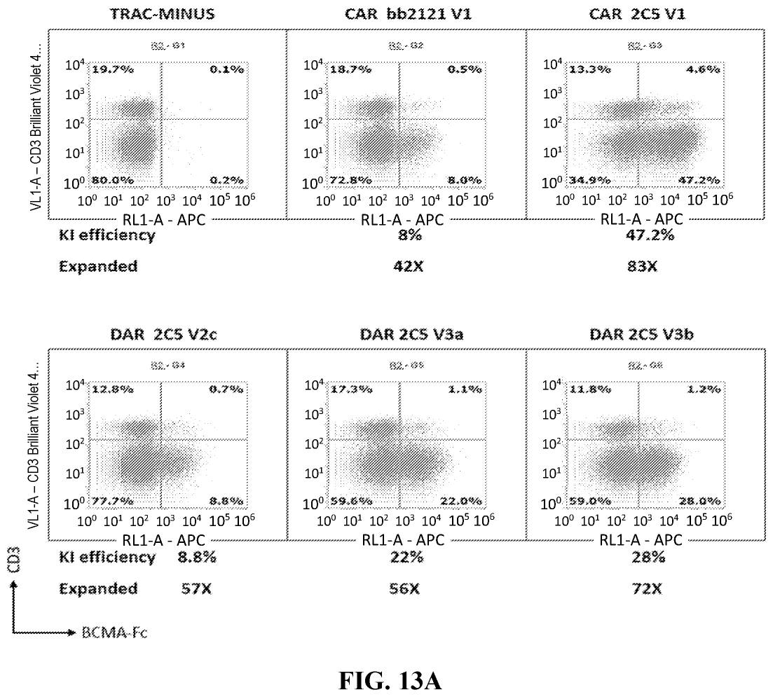

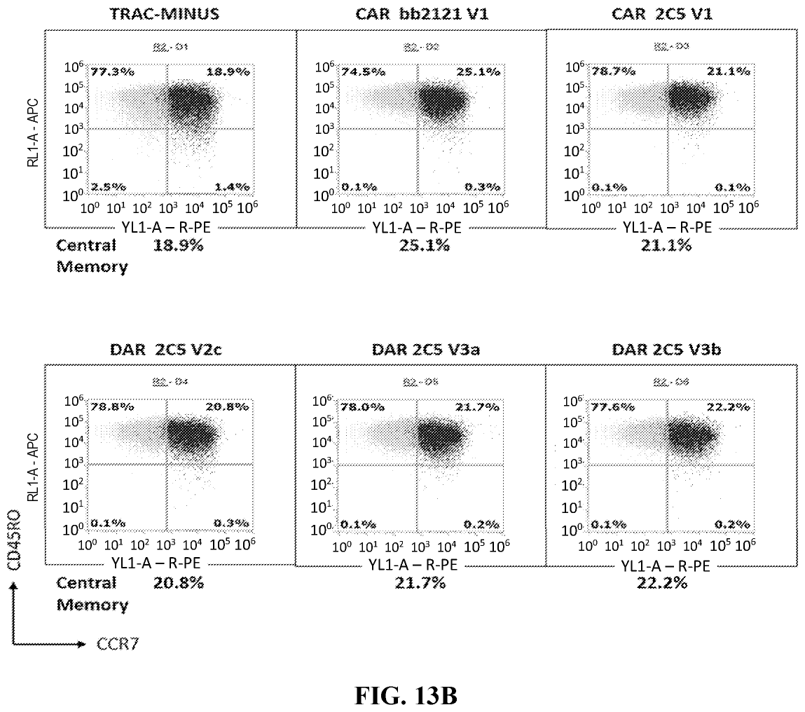

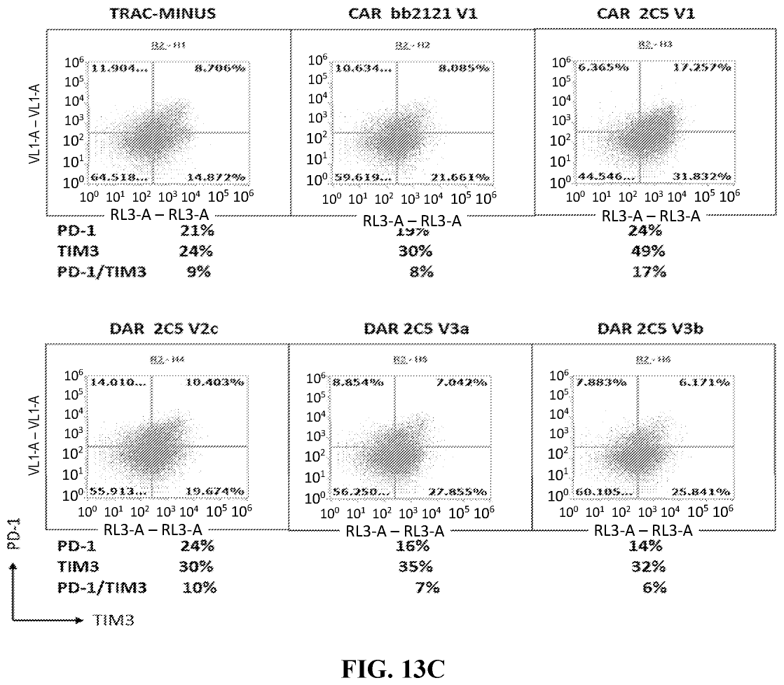

The anti-BCMA-2C5 DAR T cells (from Donor 2 cells) were washed with DPBS 5% human serum albumin, then stained with anti-CD3-BV421 antibody (SK7, BioLegend) and PE or APC conjugated BCMA-Fc protein (Chimerigen Laboratories) for 30-60 minutes at 4° C. The CD3 and BCMA were detected using iQue Screener Plus (Intellicyte Co) or Attune NxT (AFC2) (Life Technologies). Markers for identifying effector memory T cells and central memory fraction of T cells were CD45RO (BioLegend) and CCR7 (BioLegend). Central memory T cells were CD45RO and CCR7 double positive populations and effector memory T cells were CD45RO positive CCR7 negative populations. The results are shown in FIG. 13B.

### Example 10: Detecting T Cell Exhaustion Markers

The anti-BCMA 2C5 DAR T cells or the control T cells were washed with DPBS 5% human serum albumin, then stained with BV421 conjugated anti-PD1 antibody (EH12.2H7 or NAT105, from BioLegend) and APC/Cy7 conjugated TIM3 antibody (F38-2E2 from BioLegend) for 30-60 minutes at 4° C. The PD1 and TIM3 cell markers were detected using Attune NxT (AFC2) (Life Technologies). The results are shown in FIG. 13C.

### Example 11: In Vivo Tumor Killing in a Mouse Model Comparing Transgenic T Cells Expressing One of Three Different DAR Constructs

Tumoricidal activity of the anti-BCMA DAR T cells was tested in a RPMI8226 xenograft mouse model. Eight week old female NSG mice were used for the study. Multiple myeloma cell line RPMI8226 were obtained from ATCC were transfected by a lentiviral vector with luciferase and GFP genes. A single clone with luciferase and GFP expression was selected (RPMI8226-FLuc). A total 8×10⁶ cells of RPMI8226-Fluc were suspended in 200 μL PBS, and then injected intravenously into the tail vein of each mouse. Animals with very small or very large tumor burden are excluded based on the bioluminescence from IVIS imaging. The animals selected in study were randomized in different groups.

Each animal was administered a single dose of PBS, control TCR-minus T cells, or engineered anti-BCMA DAR T cells (T cells from Donor 1) via the tail vein in 200 μL of PBS on day 22 after tumor inoculation. The administered doses are listed in Table 2 below.

TABLE 2

| Group: | Group size: | Treatment: | Dose/route: |
|---|---|---|---|
| 1 | 10 | PBS | —/i.v. |
| 2 | 10 | TCR-minus | 2.5 × 10⁷ DAR + cells/i.v. |
| 3 | 10 | BCMA-2C5 DAR V2c | 2 × 10⁶ DAR + cells/i.v. |
| 4 | 10 | BCMA-2C5 DAR V3b | 4 × 10⁶ DAR + cells/i.v. |
| 5 | 10 | BCMA-2C5 DAR V3a | 4 × 10⁶ DAR + cells/i.v. |

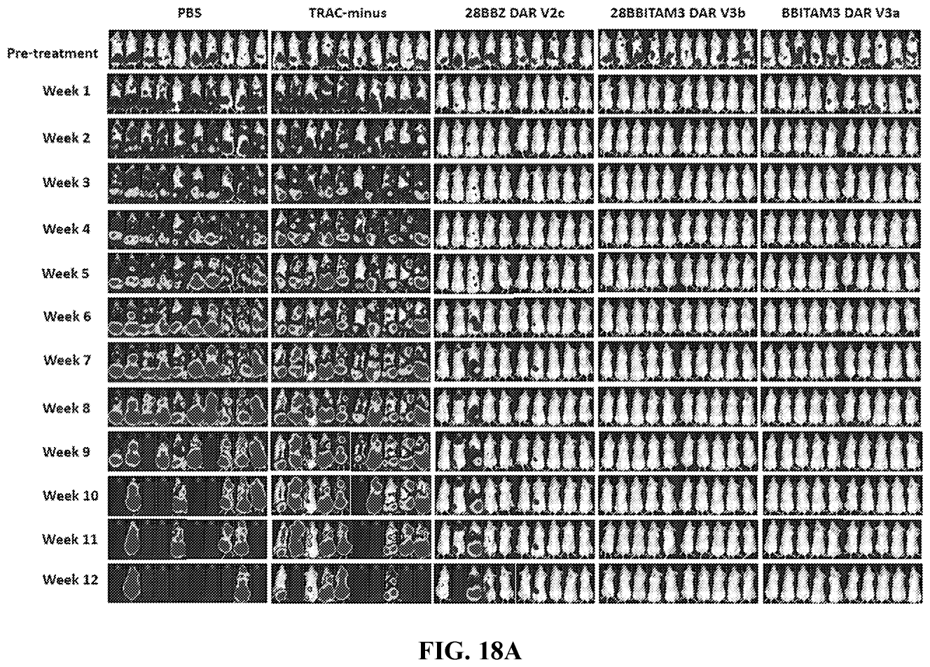

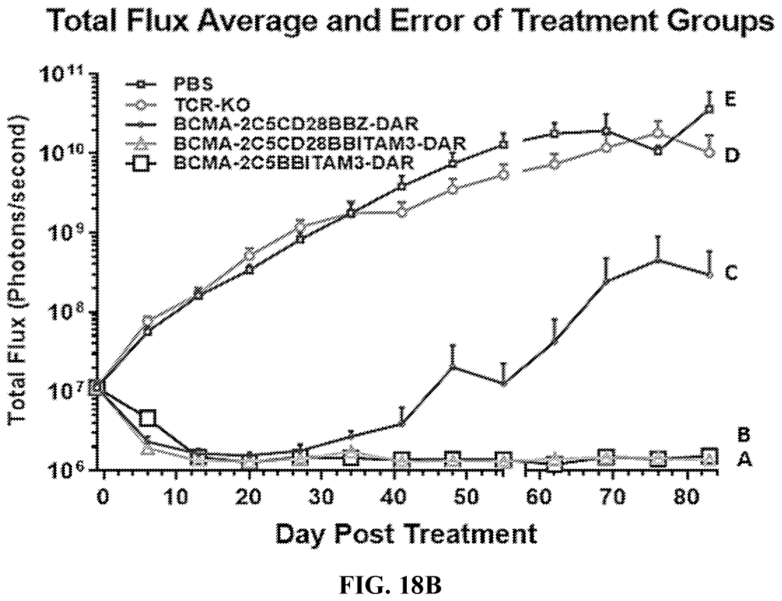

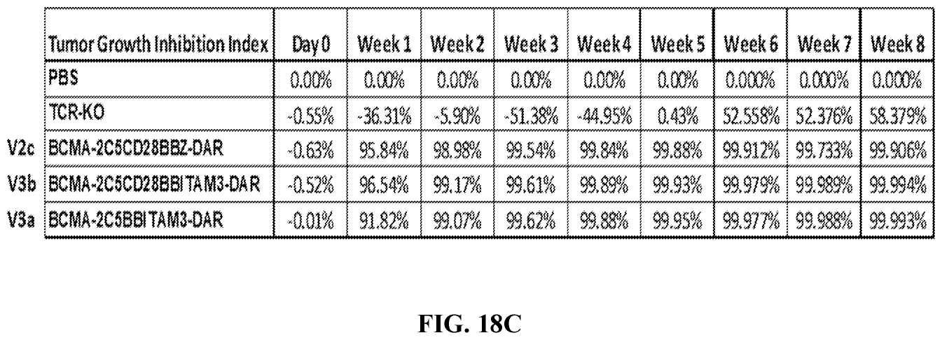

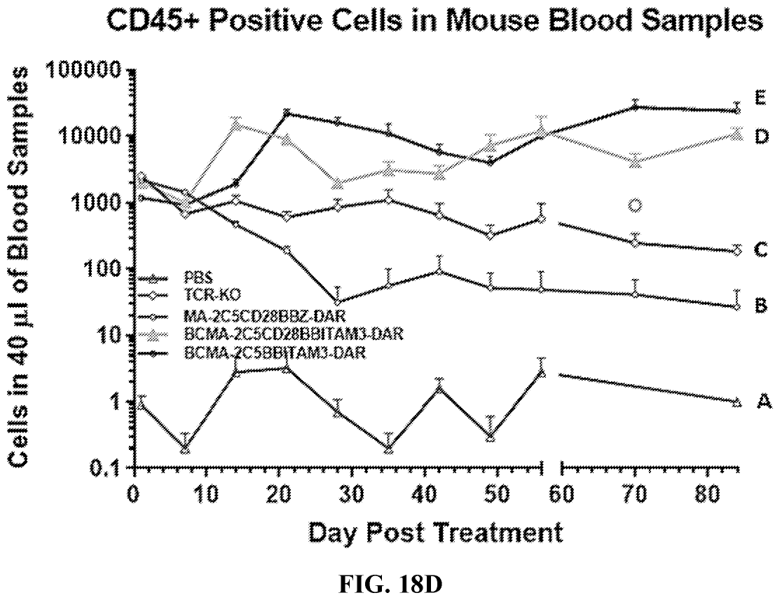

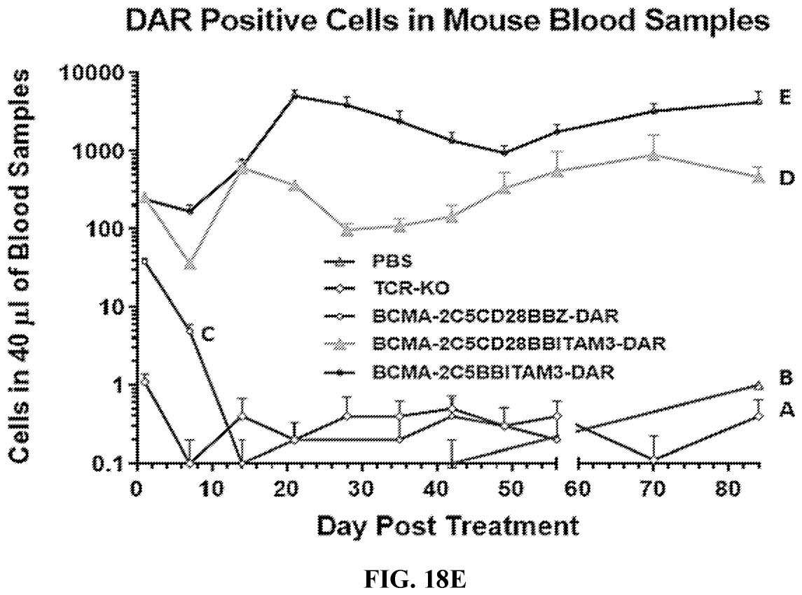

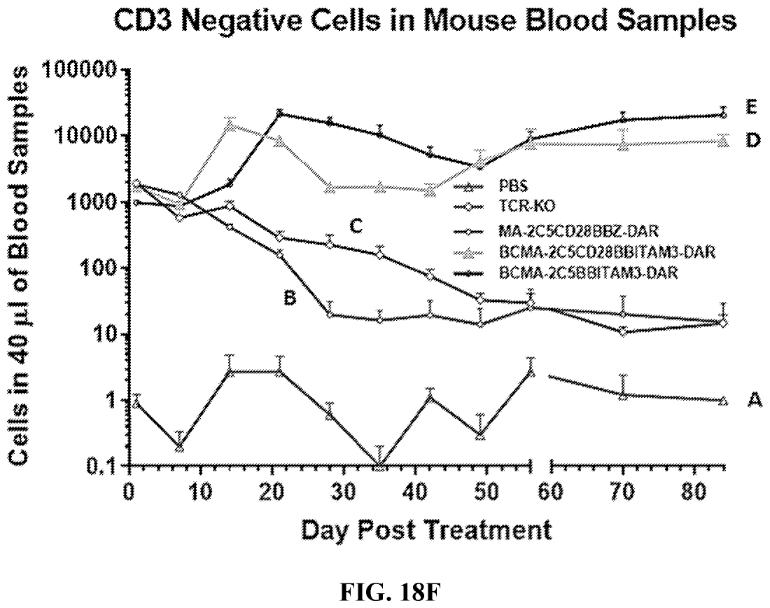

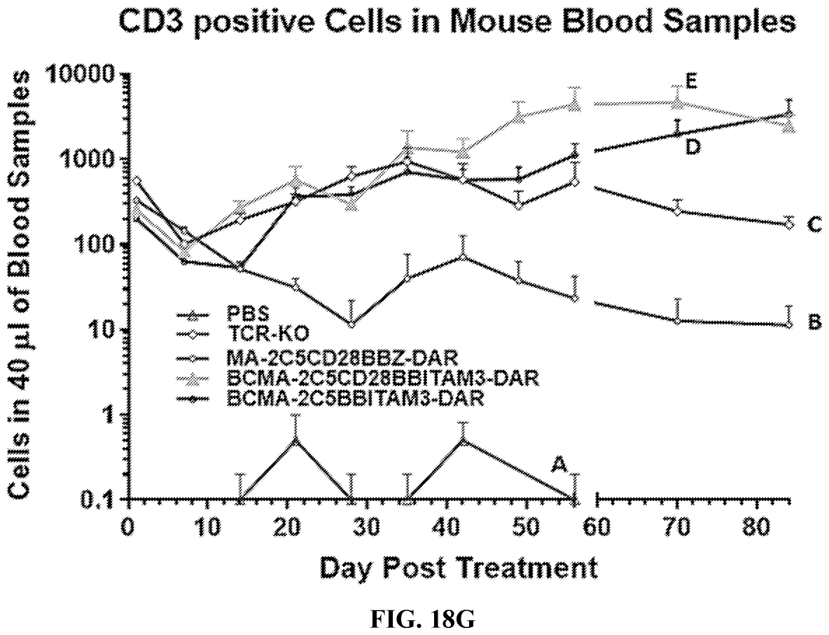

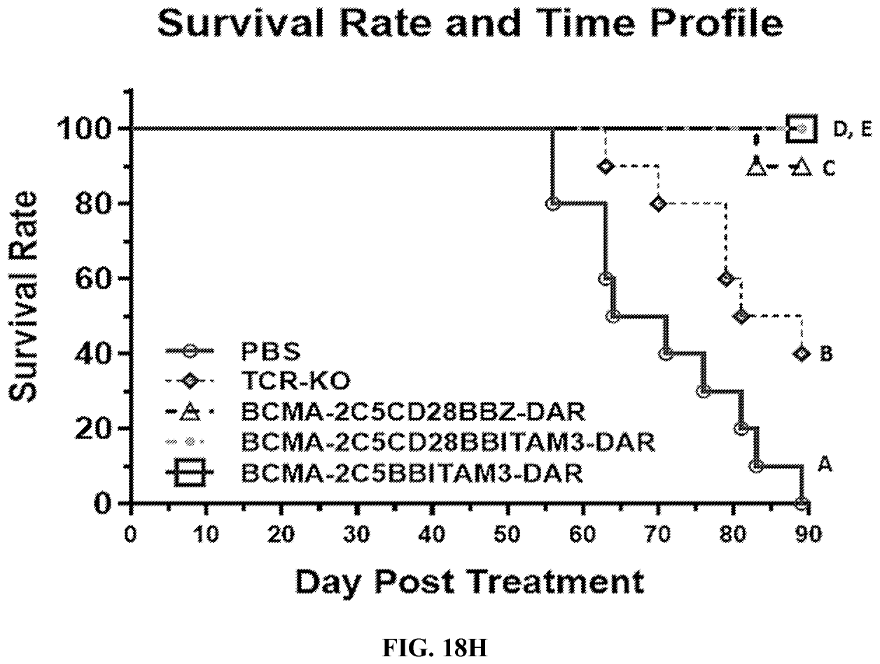

Tumor growth was monitored by measuring total photon flux with an IVIS Lumina III In Vivo Imaging System (Perkin Elmer Health Sciences, Inc) on the dorsal side of each mouse weekly after tumor cell inoculation. At week 1 post tumor inoculation, the mice that were treated with T cells expressing BCMA-2C5 DAR V2c, V3b or V3a constructs exhibited notably reduced tumor burden compared to mice treated with TCR-minus T cells or PBS (FIG. 18A). FIG. 18B is a graph showing the bioluminescent signal flux (averaged for each group of mice) corresponding to the luminescent data shown in FIG. 18A. FIG. 18C is a table listing the tumor growth inhibition index corresponding to the luminescent data shown in FIG. 18A.

Peripheral Blood FACS Analysis:

Blood samples were collected from each animal at day 1 after administration of the dose and weekly thereafter. 40 uL blood samples were obtained from the mouse tail vein. The cells from the blood samples were stained and analyzed via flow cytometry for percent and total number of CD45-positive cells (FIG. 18D), BCMA DAR-positive cells (FIG. 18E), CD3-negative cells (FIG. 18F), and CD3-positive cells (FIG. 18G). Animal survival rates were also determined (FIG. 18H).

### Example 12: In Vivo Tumor Killing in a Mouse Model Comparing Three Different Doses of Transgenic T Cells Expressing V3a DAR Construct

Tumoricidal activity of three different doses of anti-BCMA DAR T cells expressing DAR BCMA-2C5 V3a construct was tested in a RPMI8226 xenograft mouse model. Eight week old female NSG mice were used for the study. Multiple myeloma cell line RPMI8226 were obtained from ATCC were transfected by a lentiviral vector with luciferase and GFP genes. A single clone with luciferase and GFP expression was selected (RPMI8226-FLuc). A total 8×10⁶ cells of RPMI8226-Fluc were suspended in 200 μL PBS, and then injected intravenously into the tail vein of each mouse. Animals with very small or very large tumor burden are excluded based on the bioluminescence from IVIS imaging. The animals selected in study were randomized in different groups.

Each animal was administered a single dose of PBS, control TCR-minus T cells, or one of three doses of engineered anti-BCMA DAR T cells expressing the DAR BCMa-2C5 V3a construct, via the tail vein in 200 μL of PBS on day 22 after tumor inoculation. The administered doses are listed in Table 3 below.

TABLE 3

| | Group | | |
|---|---|---|---|
| Group: | size: | Treatment: | Dose/route: |
| 1 | 10 | PBS | —/i.v. |
| 2 | 10 | TCR-minus | 3 × 10⁷ DAR + cells/i.v. |
| 3 | 10 | BCMA-2C5 | 6 × 10⁶ DAR + cells in 3 × 10⁷ total T |
| | | DAR V3a | cells/i.v. |
| 4 | 10 | BCMA-2C5 | 1.2 × 10⁶ DAR + cells in 6 × 10⁶ total T |
| | | DAR V3a | cells/i.v. |
| 5 | 10 | BCMA-2C5 | 2.4 × 10⁵ DAR + cells in 1.2 × 10⁶ total |
| | | DAR V3a | T cells/i.v. |

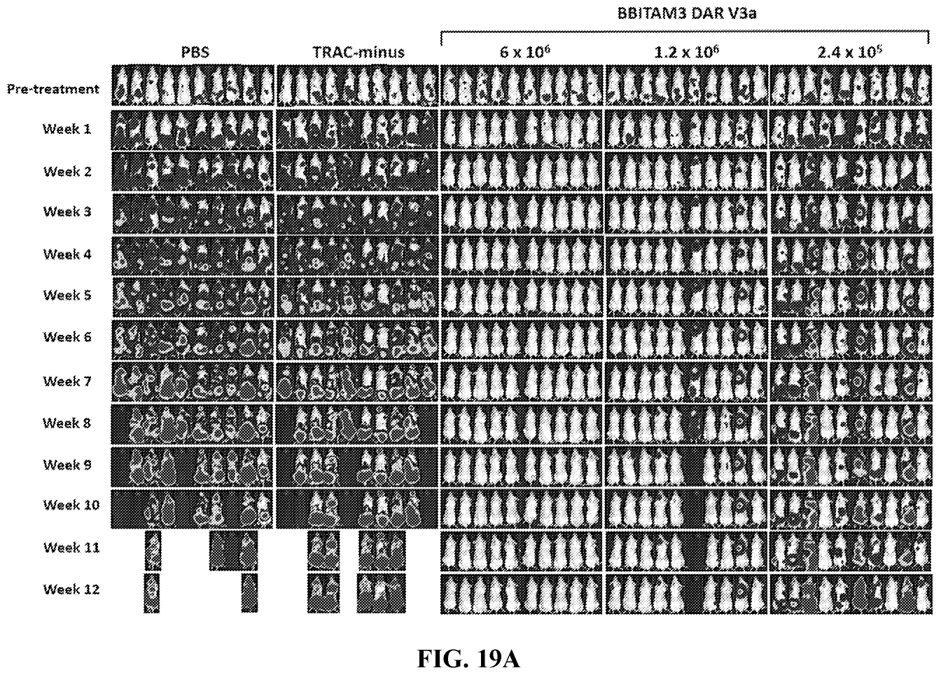

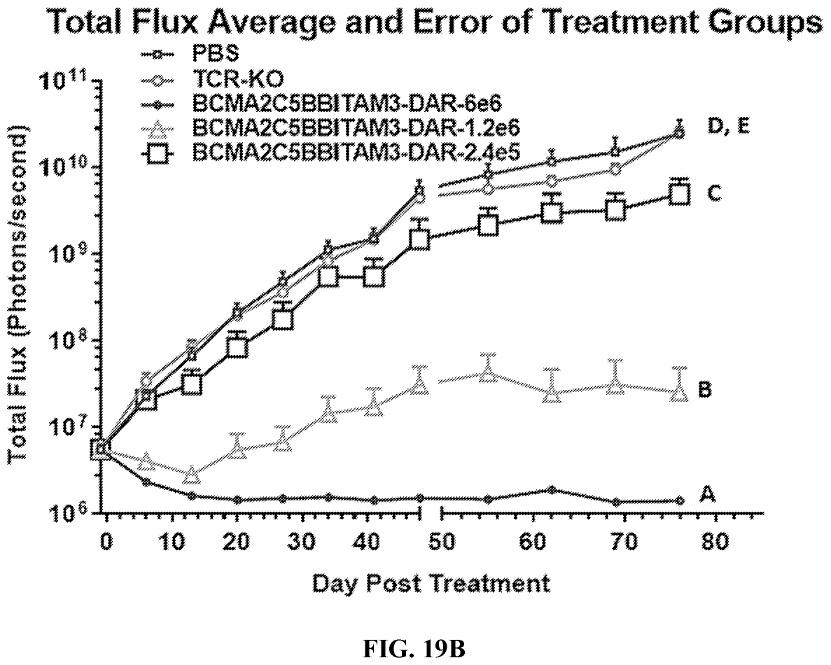

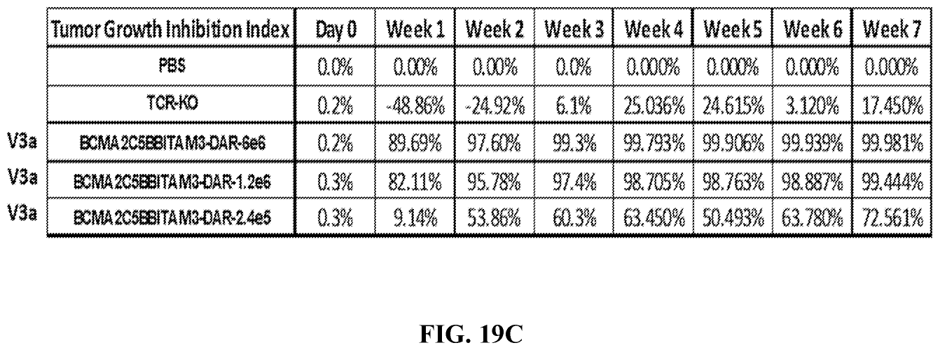

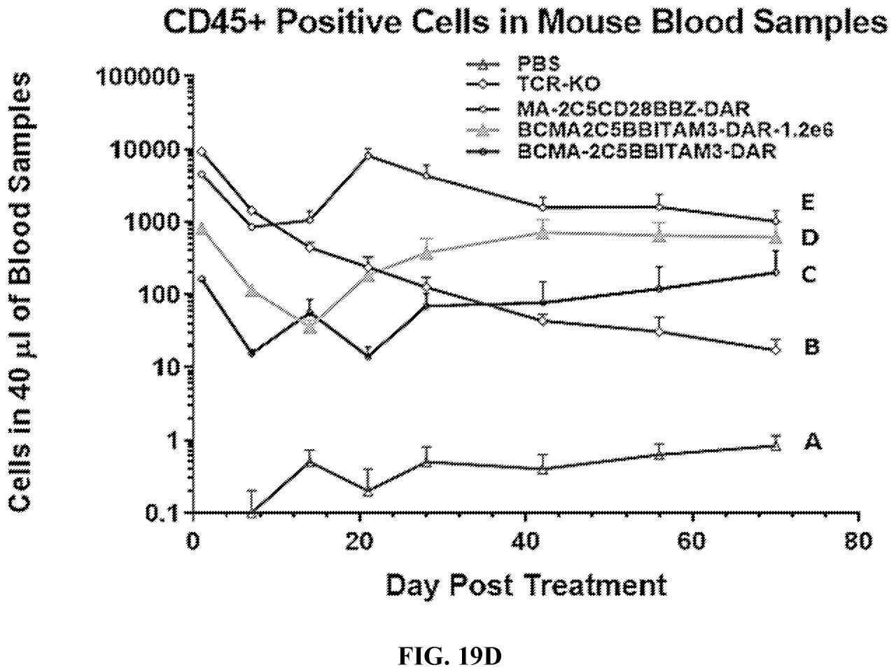

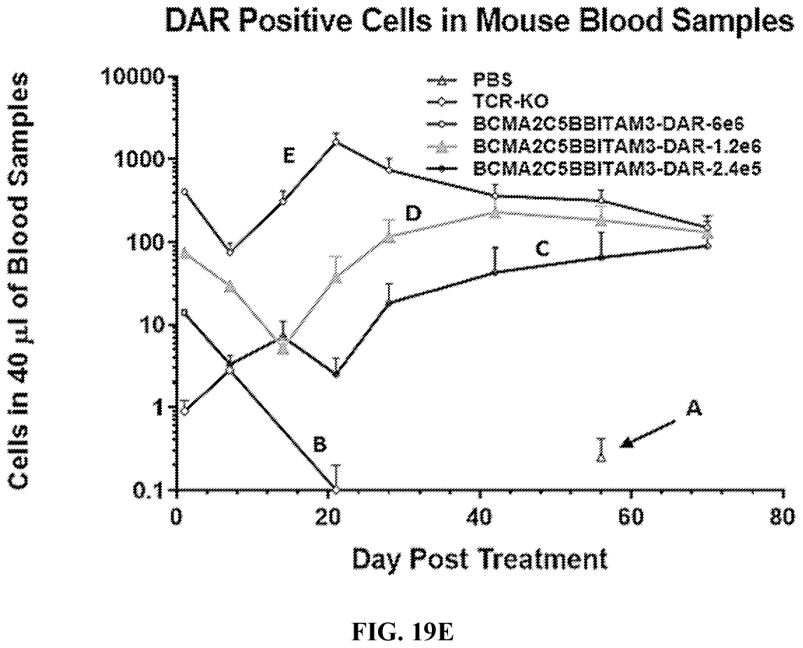

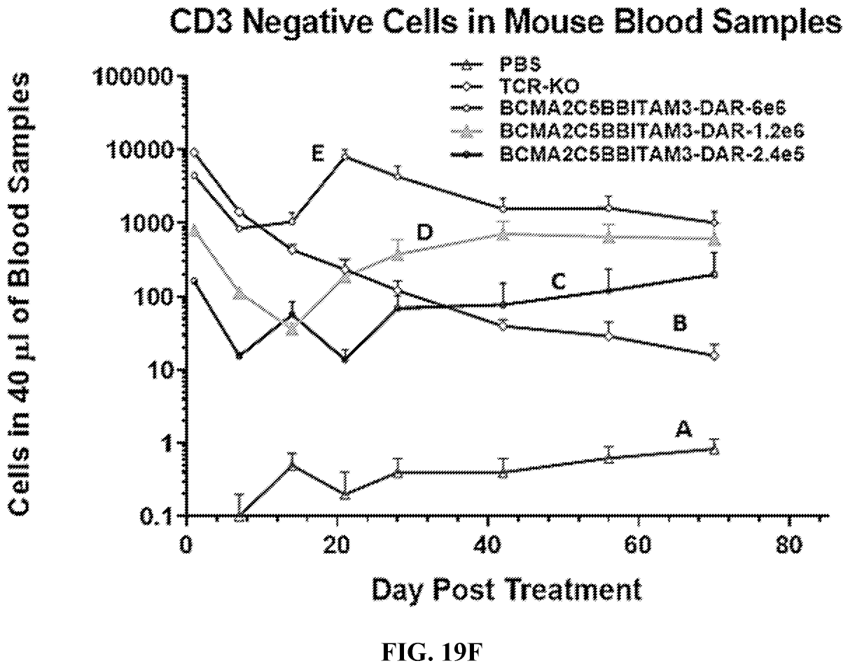

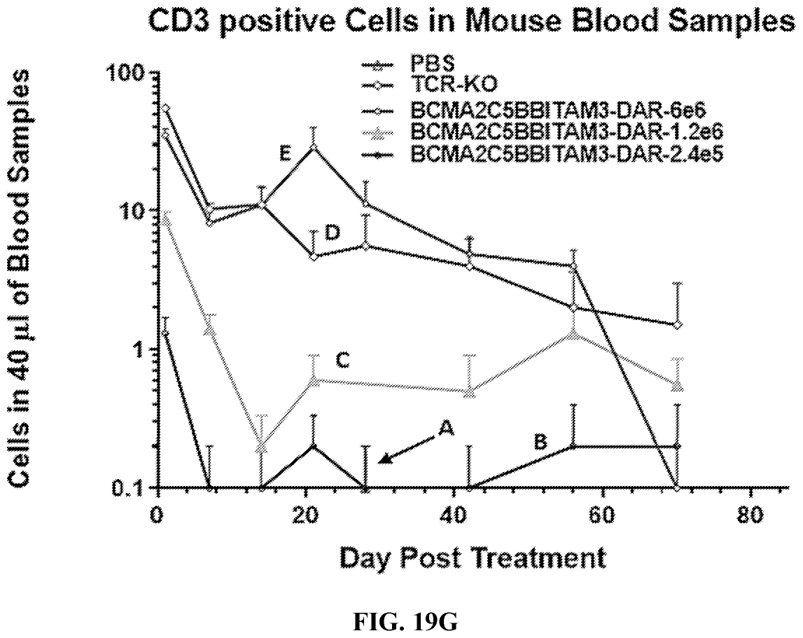

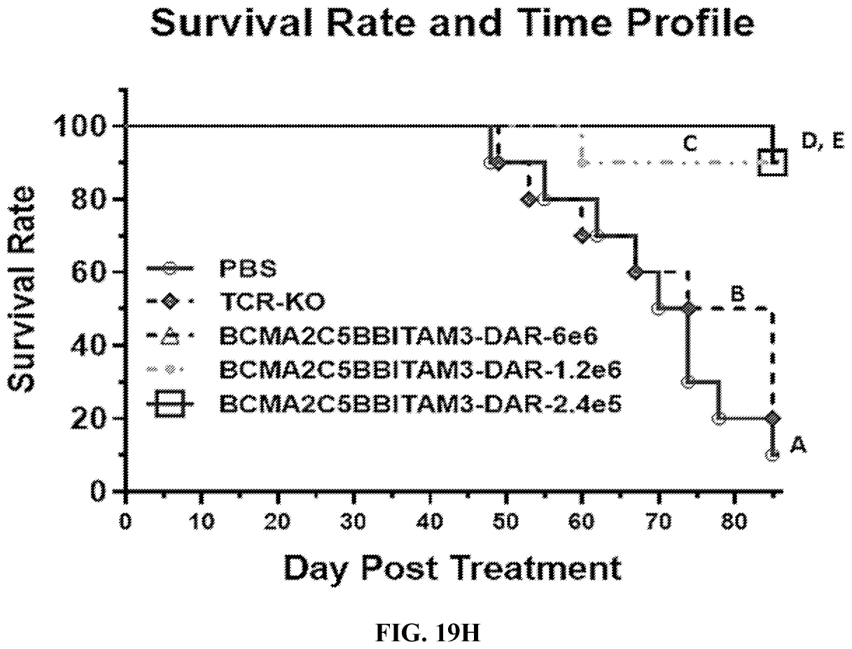

Tumor growth was monitored by measuring total photon flux with an IVIS Lumina III In Vivo Imaging System (Perkin Elmer Health Sciences, Inc) on the dorsal side of each mouse weekly after tumor cell inoculation. At week 1 and 2 post tumor inoculation, the mice that were treated with the highest dose (6×10⁶) T cells expressing BCMA-2C5 DAR V3a construct exhibited notably reduced tumor burden compared to mice treated with the moderate and lower doses (1.2×10⁶ or 2.4×10⁵) T cells expressing BCMA-2C5 DAR V3a construct (FIG. 19A). FIG. 19B is a graph showing the bioluminescent signal flux (averaged for each group of mice) corresponding to the luminescent data shown in FIG. 19A. FIG. 19C is a table listing the tumor growth inhibition index corresponding to the luminescent data shown in FIG. 19A.

Peripheral Blood PACS Analysis:

Blood samples were collected from each animal at day 1 after administration of the dose and weekly thereafter. 40 uL blood samples were obtained from the mouse tail vein. The cells from the blood samples were stained and analyzed via flow cytometry for percent and total number of CD45-positive cells (FIG. 19D), BCMA DAR-positive cells (FIG. 19E), CD3-negative cells (FIG. 19F), and CD3-positive cells (FIG. 19G). Animal survival rates were also determined (FIG. 19H).

### Example 13: In Vivo Tumor Re-Challenge Study

The mice used for the dose study described in Example 12 above were used for a tumor re-challenge study. In each group of mice that were treated with DAR T cells (V3a), half were administered 200 uL of PBS and the other half were re-challenged by administering 1×10⁷ RPMI8226-Fluc in 200 uL. In this re-challenge study, none of the mice received a second dose of DAR T cells (V3a).

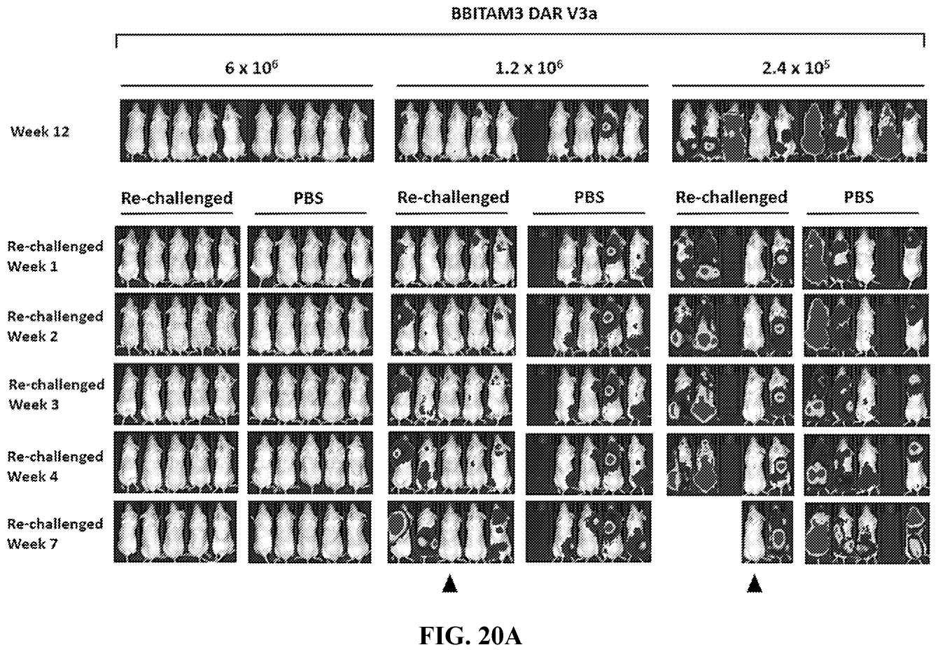

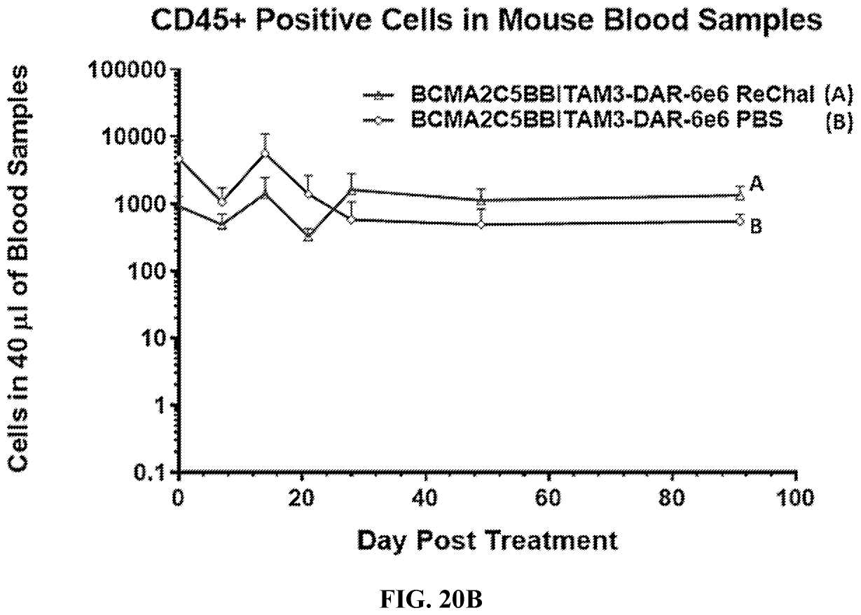

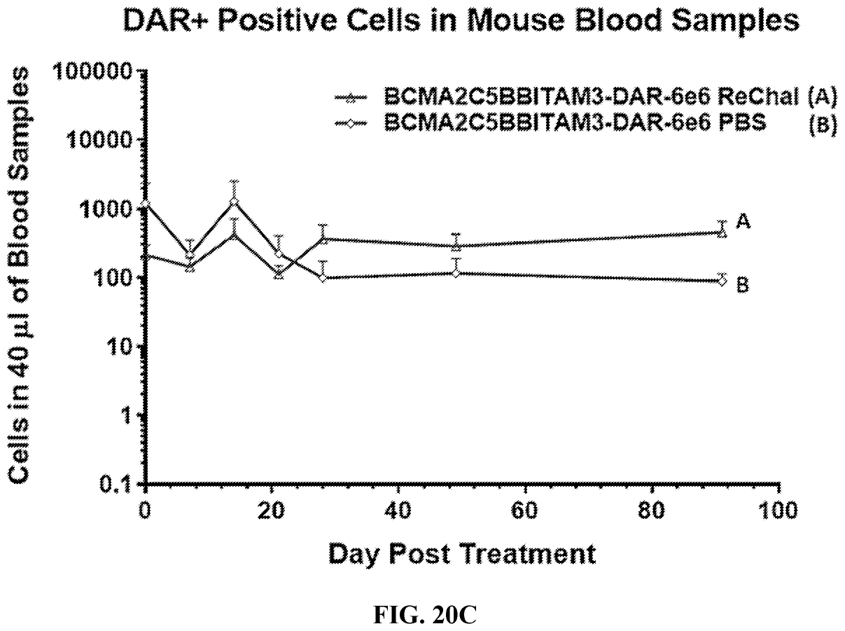

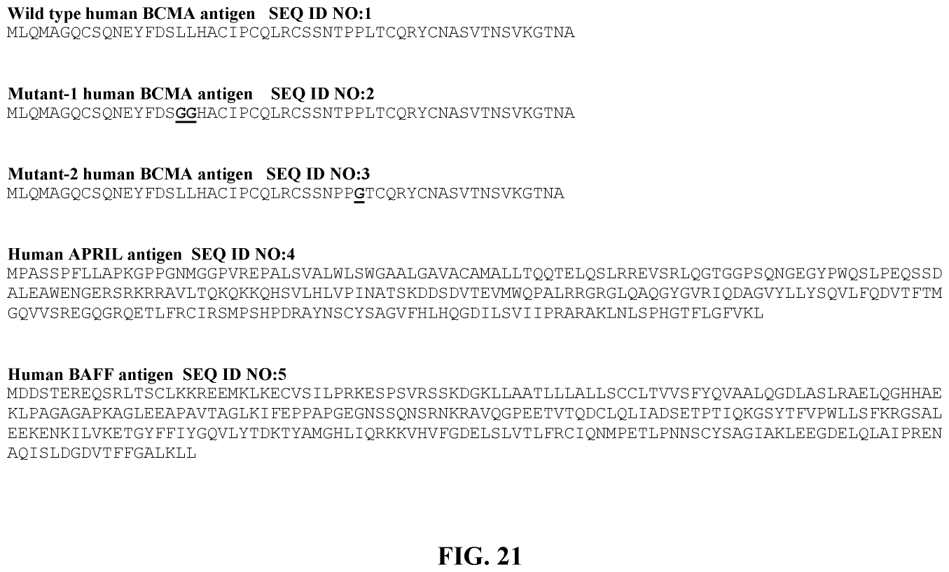

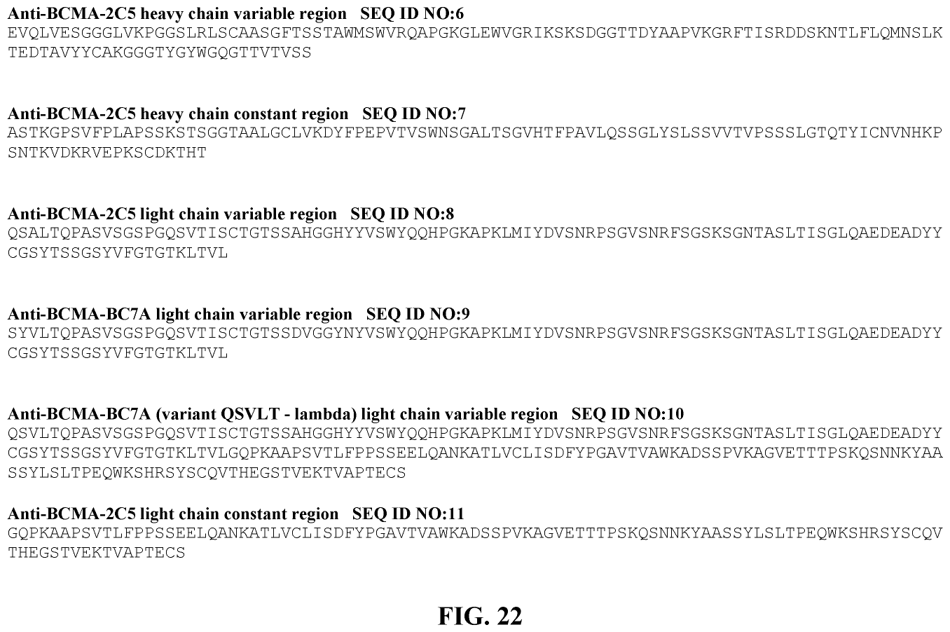

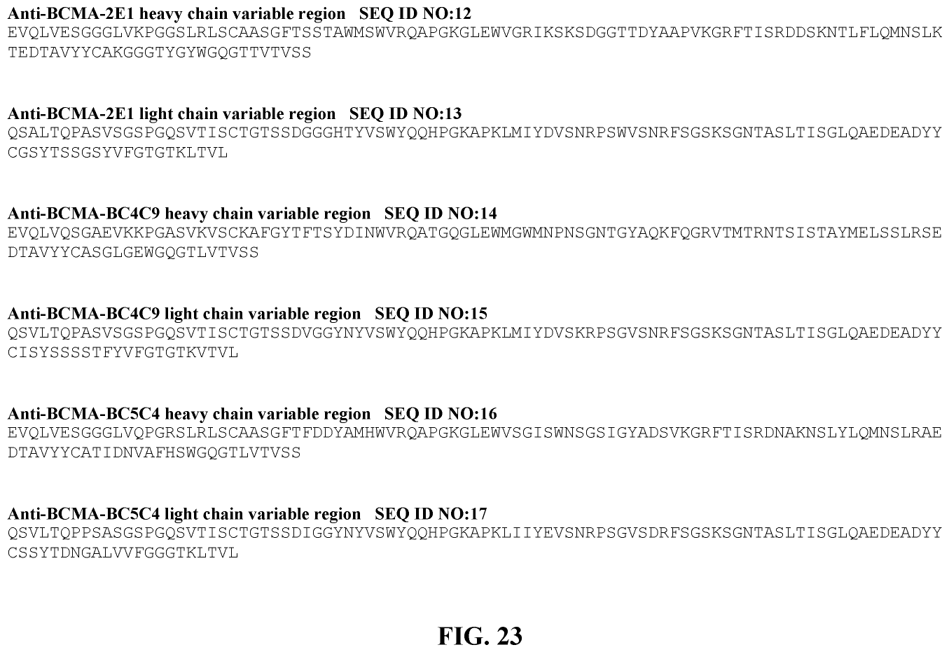

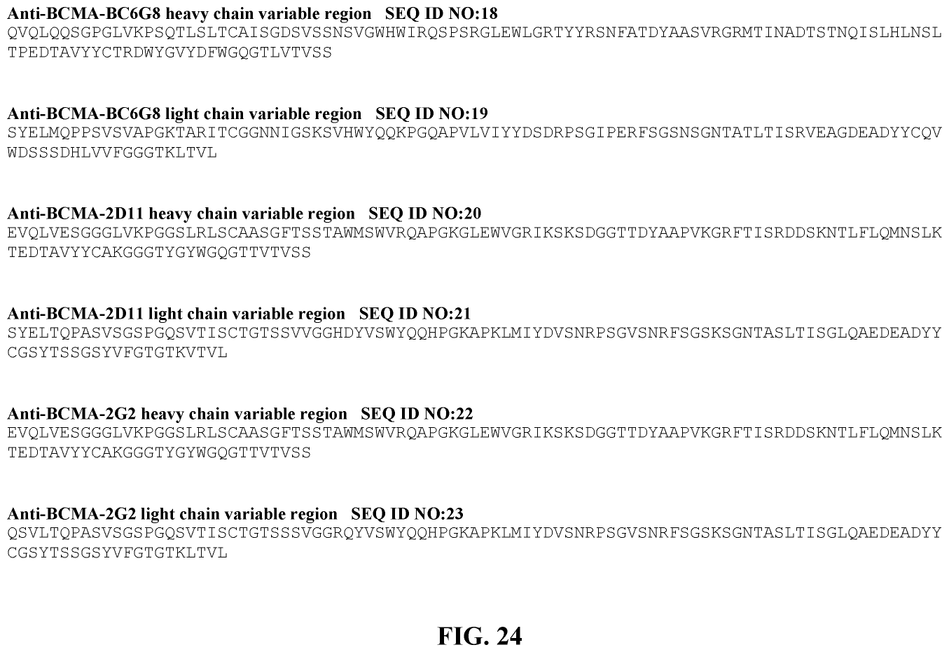

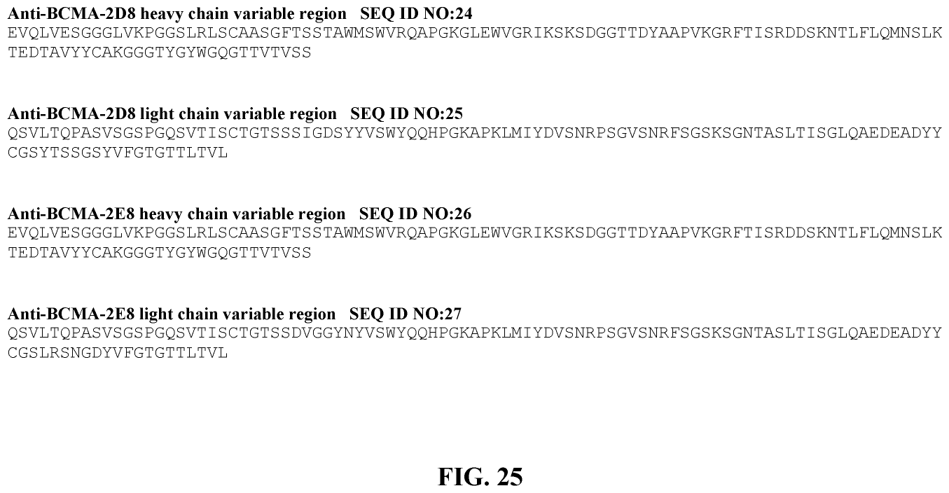

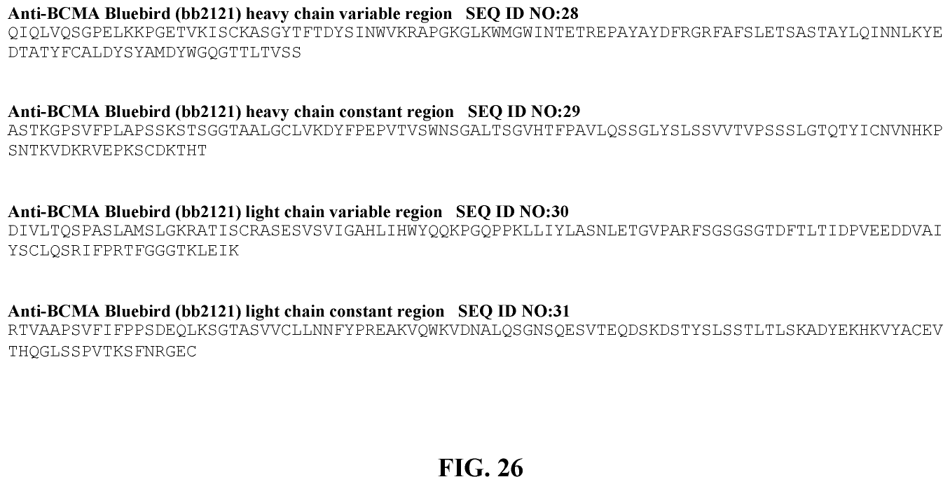

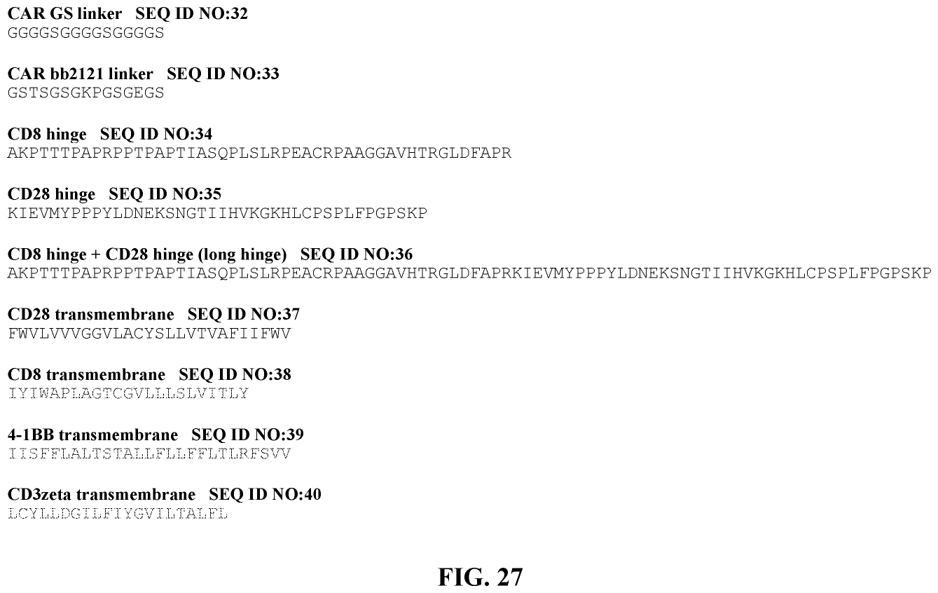

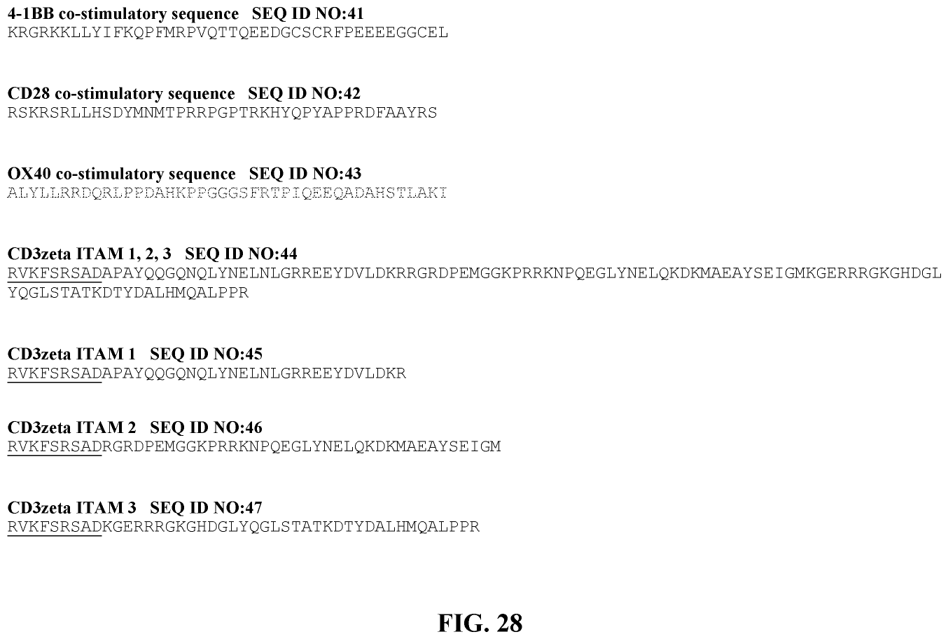

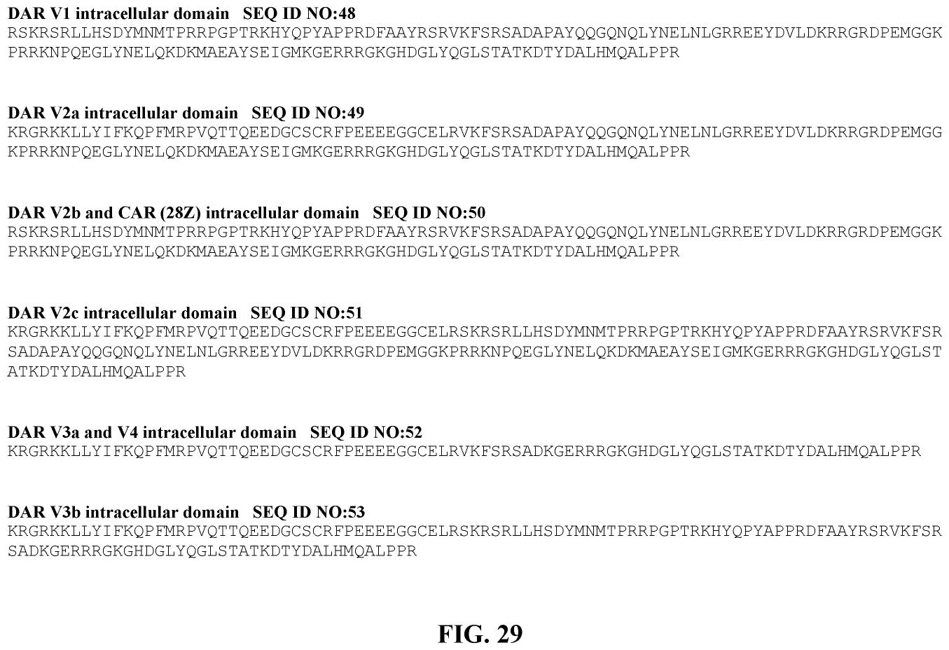

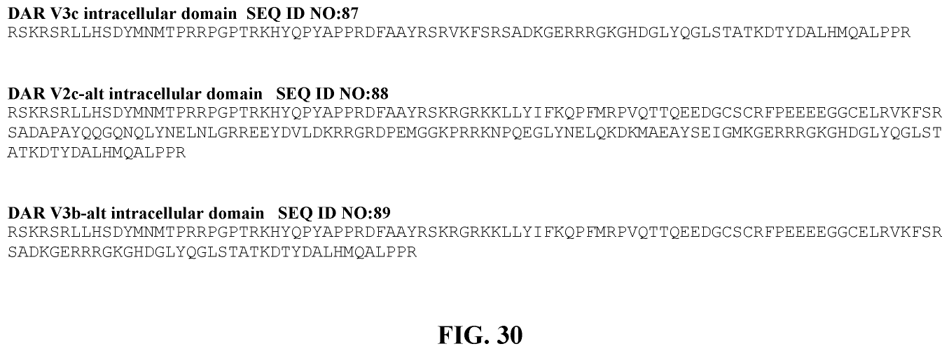

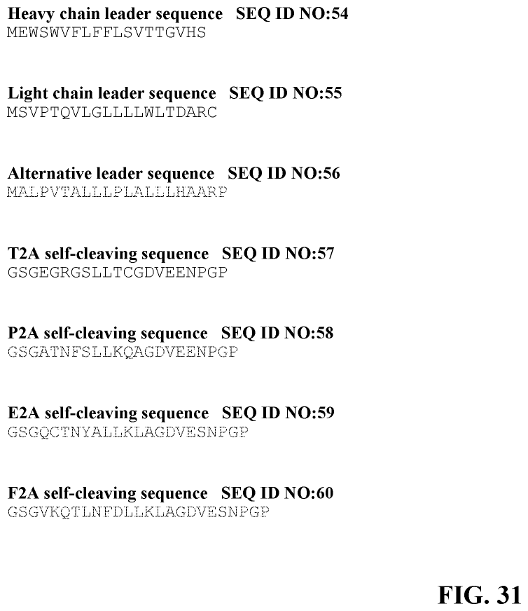

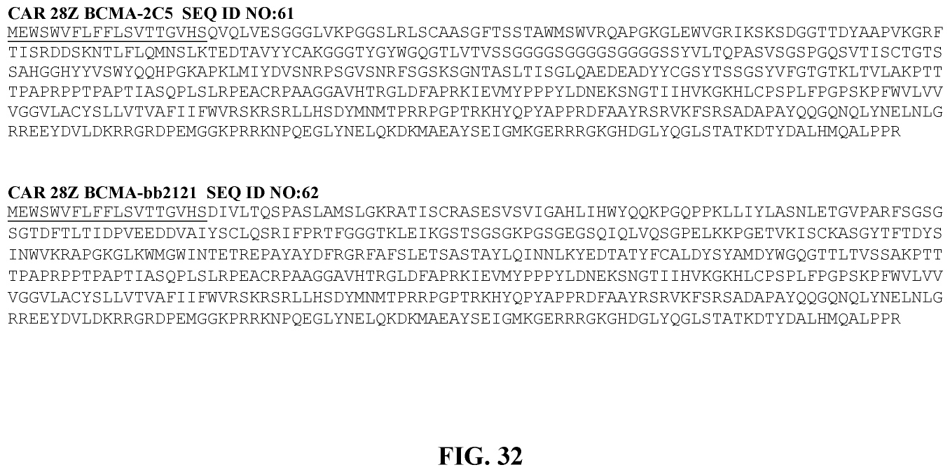

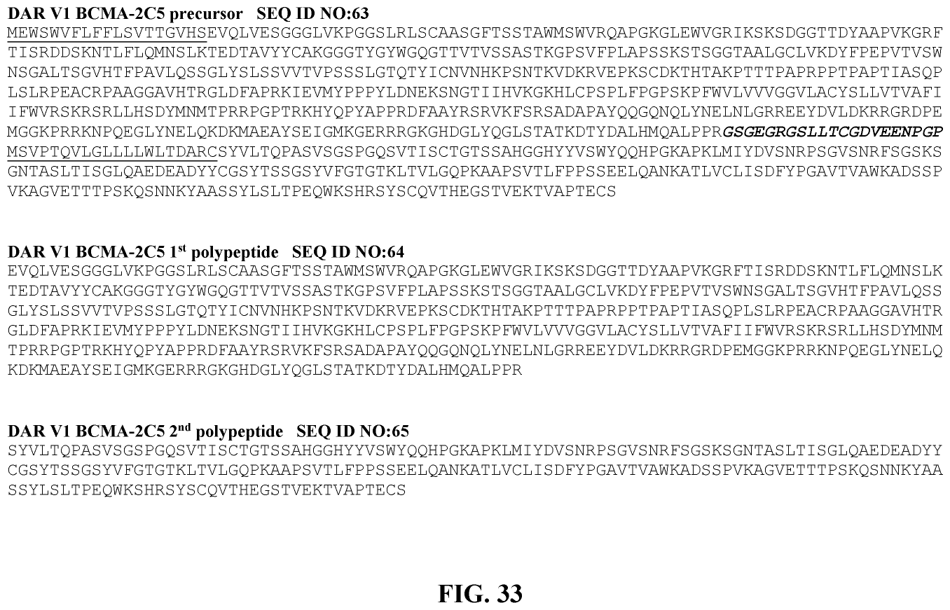

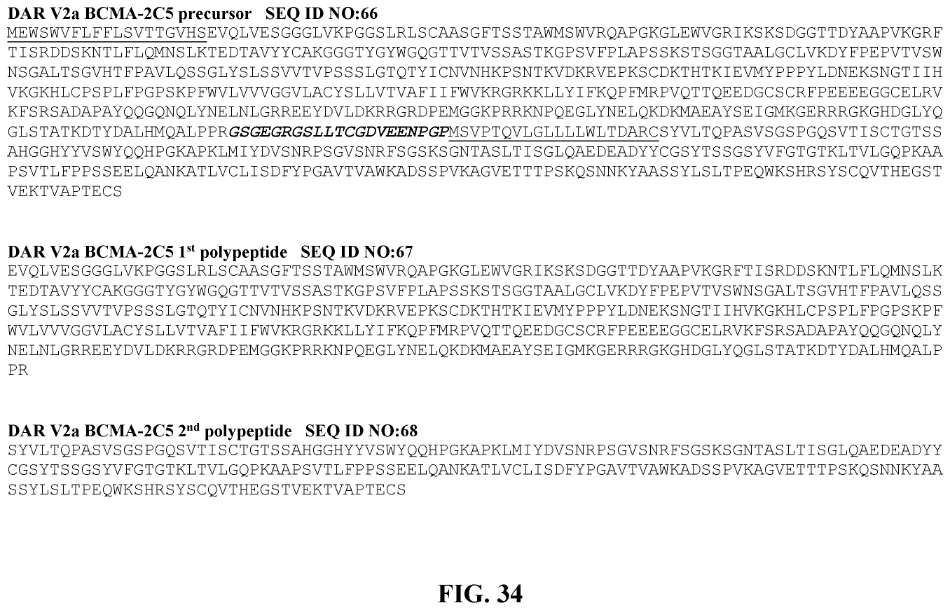

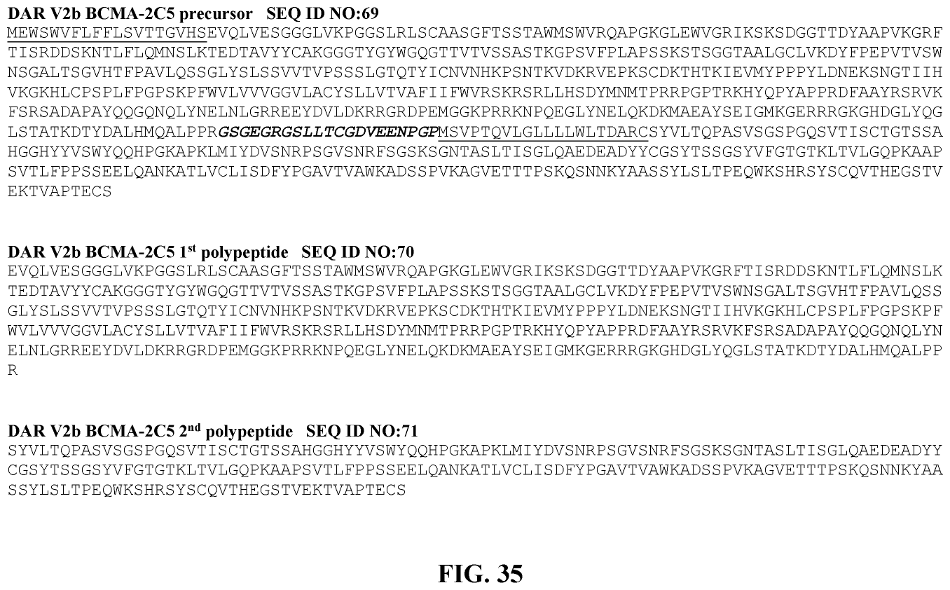

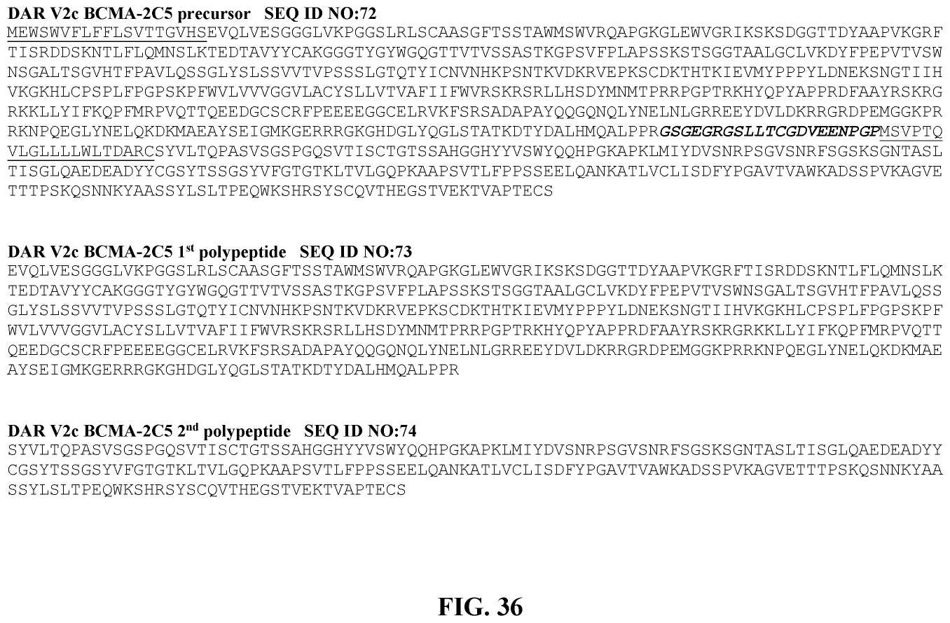

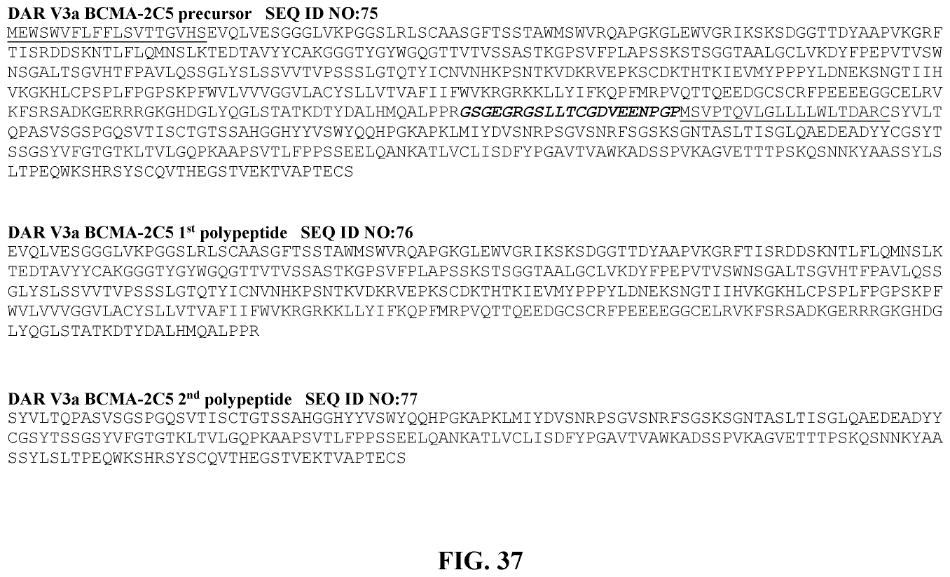

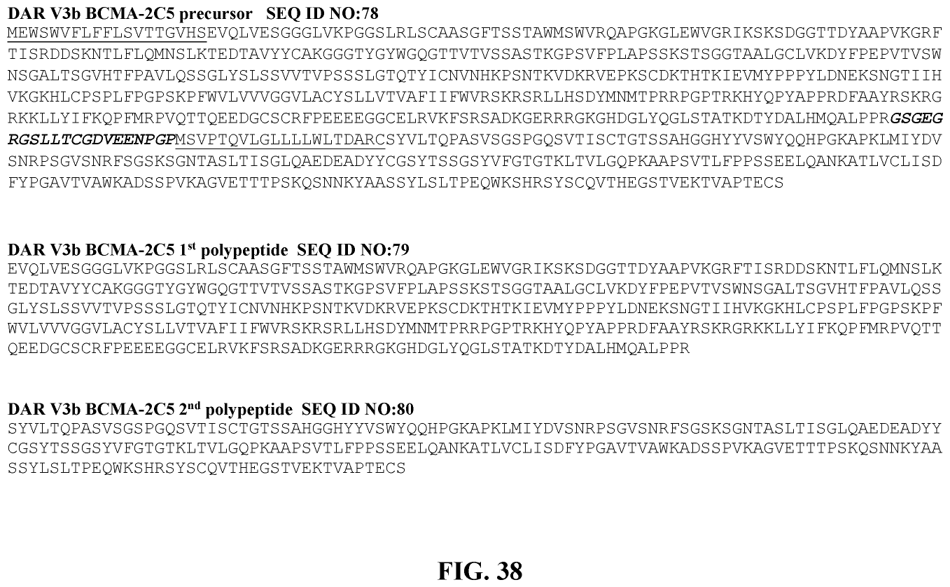

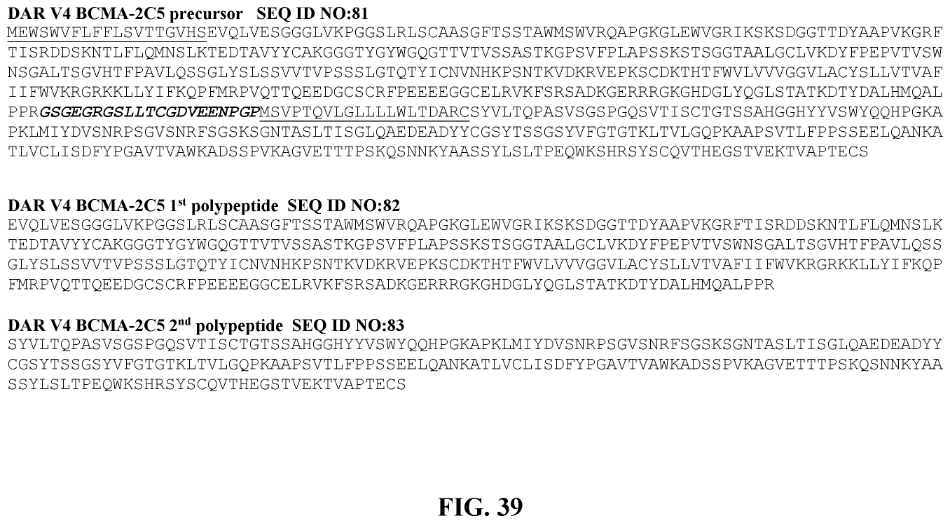

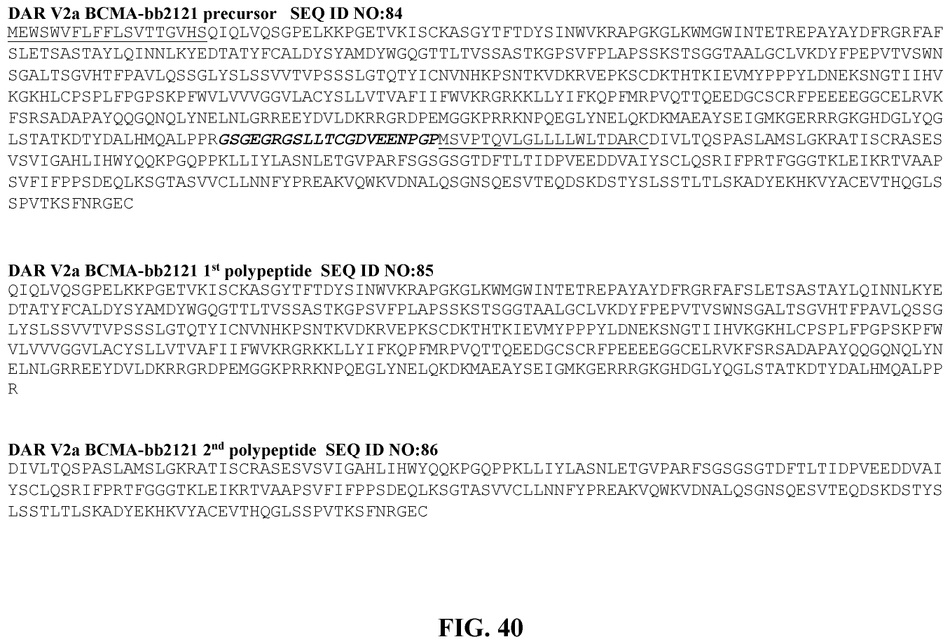

Tumor growth and re-growth was monitored by measuring total photon flux with an IVIS Lumina III In Vivo Imaging System (Perkin Elmer Health Sciences, Inc) on the dorsal side of each mouse weekly for 7 weeks. The bioluminescent images of the mice at week 12 before commencement of the tumor re-challenge study is shown at the top of FIG. 20A. Images of the mice subjected to PBS or tumor re-challenge study, for each dose group, is shown in FIG. 20A. No tumor growth was detected in the highest dosed mice (6×10⁶ DAR T cells V3a) that were subjected to tumor re-challenge (FIG. 20A). Tumor growth and re-growth was detected in four of the moderate dosed mice (1.2×10⁶) that were subjected to tumor re-challenge, and one mouse exhibited no tumor growth (indicated by a solid black triangle) (FIG. 20A). Tumor growth and re-growth was detected in four of the lowest dosed mice (2.4×10⁵) that were subjected to tumor re-challenge (three of these mice died), and one mouse exhibited no tumor growth (indicated by a solid black triangle) (FIG. 20A).

Peripheral Blood FACS Analysis:

Blood samples were collected from each animal at day 1 after administration of the dose and weekly thereafter. 40 uL blood samples were obtained from the mouse tail vein. The cells from the blood samples were stained and analyzed via flow cytometry for percent and total number of CD45-positive cells (FIG. 20B), and BCMA DAR-positive cells (FIG. 20C).

What is claimed:

1. A precursor polypeptide comprising a plurality of regions ordered from the amino terminus to the carboxyl terminus: (i) a heavy chain leader sequence, (ii) an antibody heavy chain variable region, (iii) an antibody heavy chain constant region, (iv) an optional hinge region, (v) a transmembrane region, (vi) an intracellular region, (vii) a self-cleaving sequence, (viii) a light chain leader sequence, (ix) an antibody light chain variable region, and (x) an antibody light chain constant region, wherein the self-cleaving sequence permits cleaving the of the precursor polypeptide into a first and second polypeptide chain.

2. A precursor polypeptide comprising a plurality of regions ordered from the amino terminus to the carboxyl terminus: (i) a light chain leader sequence (ii) an antibody light chain variable region, (iii) an antibody light chain constant region, (iv) an optional hinge region, (v) a transmembrane region, (vi) an intracellular region, (vii) a self-cleaving sequence, (viii) a heavy chain leader sequence, (ix) an antibody heavy chain variable region, and (x) an antibody heavy chain constant region, wherein the self-cleaving sequence permits cleaving the of the precursor polypeptide into a first and second polypeptide chain.

3. The precursor polypeptide of claim 1 or 2, wherein the antibody heavy chain variable region comprises the amino acid sequence of SEQ ID NO: 6, 12, 14, 16, 18, 20, 22, 24, 26, or 28.

4. The precursor polypeptide of any one of claims 1-3, wherein the antibody heavy chain constant region comprises:
a) a human IgG, IgA, IgD, IgE, or IgM CH1 domain;
b) a human IgG1, IgG2, IgG3, or IgG4 CH1 domain;
c) a human IgG1 domain;
d) an amino acid sequence having at least 95% sequence identity to the amino acid sequence of SEQ ID NO:7 or 29; or
e) the amino acid sequence of SEQ ID NO:7 or 29.

5. The precursor polypeptide of any one of claims 1-4, wherein the antibody light chain variable region comprises the amino acid sequence of SEQ ID NO: 8, 9, 10, 13, 15, 17, 19, 21, 23, 25, 27 or 30.

6. The precursor polypeptide of any one of claims 1-5, wherein the antibody light chain constant region comprises:
a) a human Ig kappa constant domain;
b) a human Ig lambda constant domain;
c) an amino acid sequence having at least 95% sequence identity to the amino acid sequence of SEQ ID NO:11 or 31; or
d) the amino acid sequence of SEQ ID NO:11 or 31.

7. The precursor polypeptide of any one of claims 1-6, wherein the hinge region comprises a hinge sequence from an antibody selected from a group consisting of IgG, IgA, IgM, IgE and IgD.

8. The precursor polypeptide of any one of claims 1-6, wherein the hinge comprises a CD8α and/or CD28 hinge region.

9. The precursor polypeptide of any one of claims 1-6, wherein the hinge region comprises a CPPC or SPPC amino acid sequence.

10. The precursor polypeptide of any one of claims 1-6, wherein the hinge region comprises the amino acid sequence of SEQ ID NO:34, 35 or 36.

11. The precursor polypeptide of any one of claims 1-10, wherein the transmembrane region comprises a transmembrane sequence from CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD40L/CD154, VEGFR2, FAS, and FGFR2B.

12. The precursor polypeptide of any one of claims 1-11, wherein the transmembrane region comprises the amino acid sequence of SEQ ID NO: 37, 38, 39 or 40.

13. The precursor polypeptide of any one of claims 1-12, wherein the intracellular region comprises one intracellular sequence or comprises 2-5 intracellular sequences in any order and any combination of intracellular sequences selected from a group consisting of 4-1BB, CD3zeta having ITAM 1, 2 and 3, CD3zeta having ITAM 1, CD3zeta having ITAM 3, CD28, CD27, OX40, CD30, CD40, PD-1, ICOS, lymphocyte function-associated antigen-1 (LFA-1), CD2, CD7, LIGHT, NKG2C, B7-H3, GITR (TNFRSF18), DR3 (TNFRSF25), TNFR2 and/or CD226.

14. The precursor polypeptide of any one of claims 1-12, wherein the intracellular region comprises:
i) a CD3zeta having ITAM 1, 2 and 3 which comprises the amino acid sequence of SEQ ID NO:44,
ii) a CD3zeta ITAM 1 which comprises the amino acid sequence of SEQ ID NO:45,
iii) a CD3zeta ITAM 2 which comprises the amino acid sequence of SEQ ID NO:46, or
iv) a CD3zeta having ITAM 3 which comprises the amino acid sequence of SEQ ID NO:47.

15. The precursor polypeptide of any one of claims 1-12, wherein the intracellular region comprises:
i) intracellular sequences from CD28 and from CD3zeta having ITAM 1, 2 and 3, comprising the amino acid sequence of SEQ ID NO:48 or 50, or
ii) intracellular sequences from 4-1BB and from CD3zeta having ITAM 1, 2 and 3, comprising the amino acid sequence of SEQ ID NO:49, or
iii) intracellular sequences from CD28, from 4-1BB and from CD3zeta having ITAM 1, 2 and 3, comprising the amino acid sequence of SEQ ID NO:51 or 88, or
iv) intracellular sequences from 4-1BB and from CD3zeta having ITAM 3, comprising the amino acid sequence of SEQ ID NO:52, or
v) intracellular sequences from CD28 (SEQ ID NO:42) and from CD3zeta having ITAM 3 (SEQ ID NO:47), or
vi) intracellular sequences from CD28, from 4-1BB and from CD3zeta having ITAM 3, comprising the amino acid sequence of SEQ ID NO:53 or 89.

16. The precursor molecule of claim 1, comprising the amino acid sequence of SEQ ID NO:63, 66, 69, 72, 75, 78, 81 or 84.

17. The precursor molecule of claim 2, comprising the orientation and amino acid sequences shown in FIGS. 4A and B.

18. The precursor molecule of any one of the preceding claims, wherein the self-cleaving sequence is other than a T2A sequence, e.g., the self-cleaving sequence is a P2A, E2A, or F2A sequence.

19. A dimeric antigen receptor (DAR) construct, comprising:
a) a first polypeptide chain comprising a plurality of regions ordered from the amino terminus to the carboxyl terminus: (i) an antibody heavy chain variable region, (ii) an antibody heavy chain constant region, (iii) an optional hinge region, (iv) a transmembrane region, and (v) an intracellular region;
b) a second polypeptide chain comprising a plurality of regions ordered from the amino terminus to the carboxyl terminus: (i) an antibody light chain variable region, and (ii) an antibody light chain constant region,
wherein the antibody heavy chain constant region and the antibody light chain constant region form a dimerization domain for formation of the dimeric antigen receptor (DAR), and
wherein the antibody heavy chain variable region and the antibody light chain variable region form an antigen binding domain.

20. A dimeric antigen receptor (DAR) construct, comprising:
a) a first polypeptide chain comprising a plurality of regions ordered from the amino terminus to the carboxyl terminus: (i) an antibody light chain variable region, (ii) an antibody light chain constant region, (iii) an optional hinge region, (iv) a transmembrane region, and (v) an intracellular region;
b) a second polypeptide chain comprising a plurality of regions ordered from the amino terminus to the carboxyl terminus: (i) an antibody heavy chain variable region, and (ii) an antibody heavy chain constant region,
wherein the antibody heavy chain constant region and the antibody light chain constant region form a dimerization domain for formation of the dimeric antigen receptor (DAR), and
wherein the antibody heavy chain variable region and the antibody light chain variable region form an antigen binding domain.

21. The dimeric antigen receptor (DAR) construct of claim 18 or 19, wherein the antibody heavy chain constant region and the antibody light chain constant region dimerize via one or two disulfide bonds.

22. The dimeric antigen receptor construct of any one of claims 18-20, wherein the hinge region comprises a hinge sequence from an antibody selected from a group consisting of IgG, IgA, IgM, IgE and IgD.

23. The dimeric antigen receptor construct of any one of claims 18-20, wherein the hinge comprises a CD8α and/or a CD28 hinge region.

24. The dimeric antigen receptor construct of any one of claims 18-20, wherein the hinge region comprises a CPPC or SPPC amino acid sequence.

25. The dimeric antigen receptor construct of any one of claims 18-23, wherein the transmembrane region comprises a transmembrane sequence from CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD40L/CD154, VEGFR2, FAS, and FGFR2B.

26. The dimeric antigen receptor construct of any one of claims 18-24, wherein the intracellular region comprises one intracellular sequence or comprises 2-5 intracellular sequences in any order and any combination of intracellular sequences selected from a group consisting of 4-1BB, CD3zeta, CD28, CD27, OX40, CD30, CD40, PD-1, ICOS, lymphocyte function-associated antigen-1 (LFA-1), CD2, CD7, LIGHT, NKG2C, B7-H3, GITR (TNFRSF18), DR3 (TNFRSF25), TNFR2 and/or CD226.

27. The dimeric antigen receptor (DAR) construct of any one of claims 18-25, wherein the antigen binding domain binds a BMCA (B-cell maturation antigen) protein.

28. The dimeric antigen receptor (DAR) construct of claim 27, wherein the BMCA (B-cell maturation antigen) protein comprises the amino acid sequence of SEQ ID NO:1, 2 or 3.

29. The dimeric antigen receptor (DAR) construct of claim 27, wherein the antibody heavy chain variable region comprises the amino acid sequence of SEQ ID NO: 6, 12, 14, 16, 18, 20, 22, 24, 26, or 28.

30. The dimeric antigen receptor (DAR) construct of claim 27, wherein the antibody heavy chain constant region comprises the amino acid sequence of SEQ ID NO:7 or 29.

31. The dimeric antigen receptor (DAR) construct of claim 27, wherein the antibody light chain variable region comprises the amino acid sequence of SEQ ID NO: 8, 9, 10, 13, 15, 17, 19, 21, 23, 25, 27 or 30.

32. The dimeric antigen receptor (DAR) construct of claim 27, wherein the antibody light chain constant region comprises the amino acid sequence of 11 or 31.

33. The dimeric antigen receptor (DAR) construct of claim 27, wherein the hinge region comprises the amino acid sequence of SEQ ID NO:34, 35 or 36.

34. The dimeric antigen receptor (DAR) construct of claim 27, wherein the transmembrane region comprises the amino acid sequence of SEQ ID NO:37, 38, 39 or 40.

35. The dimeric antigen receptor of claim 27, wherein the intracellular region comprises:
i) a CD3zeta having ITAM 1, 2 and 3 comprising the amino acid sequence of SEQ ID NO:44,
ii) a CD3zeta ITAM 1 comprising the amino acid sequence of SEQ ID NO:45,
iii) a CD3zeta ITAM 2 comprising the amino acid sequence of SEQ ID NO:46, or
iv) a CD3zeta having ITAM 3 comprising the amino acid sequence of SEQ ID NO:47.

36. The dimeric antigen receptor of claim 27, wherein the intracellular region comprises:
i) intracellular sequences from CD28 and from CD3zeta having ITAM 1, 2 and 3, comprising the amino acid sequence of SEQ ID NO:48 or 50, or
ii) intracellular sequences from 4-1BB and from CD3zeta having ITAM 1, 2 and 3, comprising the amino acid sequence of SEQ ID NO:49, or
iii) intracellular sequences from CD28, from 4-1BB and from CD3zeta having ITAM 1, 2 and 3, comprising the amino acid sequence of SEQ ID NO:51 or 88, or
iv) intracellular sequences from 4-1BB and from CD3zeta having ITAM 3, comprising the amino acid sequence of SEQ ID NO:52, or
v) intracellular sequences from CD28 (SEQ ID NO:42) and from CD3zeta having ITAM 3 (SEQ ID NO:47), or
vi) intracellular sequences from CD28, from 4-1BB and from CD3zeta having ITAM 3, comprising the amino acid sequence of SEQ ID NO:53 or 89.

37. The dimeric antigen receptor (DAR) construct of claim 27, wherein the first polypeptide chain comprises the amino acid sequence of SEQ ID NO:64, 67, 70, 73, 76, 79, 82 or 85.

38. The dimeric antigen receptor (DAR) construct of claim 27, wherein the second polypeptide chain comprises the amino acid sequence of SEQ ID NO:65, 68, 71, 74, 77, 80, 83, or 86.

39. The precursor polypeptide of any one of claims 1-18, wherein upon cleavage of the self-cleaving sequence, the heavy chain variable region and the light chain variable region are capable of forming an antigen-binding domain that binds a BMCA (B-cell maturation antigen) protein, optionally wherein the BMCA (B-cell maturation antigen) protein comprises the amino acid sequence of SEQ ID NO:1, 2 or 3.

40. The dimeric antigen receptor (DAR) construct of claim 27, wherein
a) the first polypeptide chain comprises a plurality of regions ordered from the amino terminus to the carboxyl terminus: (i) a BCMA antibody heavy chain variable region comprising the amino acid sequence of SEQ ID NO: 6, 12, 14, 16, 18, 20, 22, 24, 26, or 28; (ii) an antibody heavy chain constant region; (iii) a hinge region comprising a CD8 and CD28 hinge region; (iv) a CD28 transmembrane region; and (v) an intracellular region comprising a CD28 co-stimulatory sequence and CD3zeta ITAM 1, 2 and 3 intracellular sequences; and wherein
b) the second polypeptide chain comprising a plurality of regions ordered from the amino terminus to the carboxyl terminus: (i) a BCMA antibody light chain variable region comprising the amino acid sequence of SEQ ID NO: 8, 9, 10, 13, 15, 17, 19, 21, 23, 25, 27 or 30; and (ii) an antibody light chain constant region,
wherein the antibody heavy chain constant region and the antibody light chain constant region form a dimerization domain, and
wherein the antibody heavy chain variable region and the antibody light chain variable region form an antigen binding domain that binds a BCMA protein, optionally wherein the dimeric antigen receptor (DAR) construct is a DAR V1 construct.

41. The dimeric antigen receptor (DAR) construct of claim 27, wherein
a) the first polypeptide chain comprises a plurality of regions ordered from the amino terminus to the carboxyl terminus: (i) a BCMA antibody heavy chain variable region comprising the amino acid sequence of SEQ ID NO:6, 12, 14, 16, 18, 20, 22, 24, 26, or 28; (ii) an antibody heavy chain constant region; (iii) a hinge region comprising a CD28 hinge region; (iv) a CD28 transmembrane region; and (v) an intracellular region comprising a 4-1BB co-stimulatory sequence and CD3zeta ITAM 1, 2 and 3 intracellular sequences; and wherein
b) the second polypeptide chain comprising a plurality of regions ordered from the amino terminus to the carboxyl terminus: (i) a BCMA antibody light chain variable region comprising the amino acid sequence of SEQ ID NO: 8, 9, 10, 13, 15, 17, 19, 21, 23, 25, 27 or 30; and (ii) an antibody light chain constant region,
wherein the antibody heavy chain constant region and the antibody light chain constant region form a dimerization domain, and
wherein the antibody heavy chain variable region and the antibody light chain variable region form an antigen binding domain that binds a BCMA protein, optionally wherein the dimeric antigen receptor (DAR) construct is a DAR V2a construct.

42. The dimeric antigen receptor (DAR) construct of claim 27, wherein
a) the first polypeptide chain comprises a plurality of regions ordered from the amino terminus to the carboxyl terminus: (i) a BCMA antibody heavy chain variable region comprising the amino acid sequence of SEQ ID NO: 6, 12, 14, 16, 18, 20, 22, 24, 26, or 28; (ii) an antibody heavy chain constant region; (iii) a hinge region; (iv) a CD28 transmembrane region; and (v) an intracellular region comprising a CD28 co-stimulatory sequence and CD3zeta ITAM 1, 2 and 3 intracellular sequences; and wherein
b) the second polypeptide chain comprising a plurality of regions ordered from the amino terminus to the carboxyl terminus: (i) a BCMA antibody light chain variable region comprising the amino acid sequence of SEQ ID NO: 8, 9, 10, 13, 15, 17, 19, 21, 23, 25, 27 or 30; and (ii) an antibody light chain constant region,
wherein the antibody heavy chain constant region and the antibody light chain constant region form a dimerization domain, and
wherein the antibody heavy chain variable region and the antibody light chain variable region form an antigen binding domain that binds a BCMA protein, optionally wherein the dimeric antigen receptor (DAR) construct is a DAR V2b construct.

43. The dimeric antigen receptor (DAR) construct of claim 27, wherein
a) the first polypeptide chain comprises a plurality of regions ordered from the amino terminus to the carboxyl terminus: (i) a BCMA antibody heavy chain variable region comprising the amino acid sequence of SEQ ID NO: 6, 12, 14, 16, 18, 20, 22, 24, 26, or 28; (ii) an antibody heavy chain constant region; (iii) a hinge region comprising a CD28 hinge region; (iv) a CD28 transmembrane region; and (v) an intracellular region comprising a 4-1BB co-stimulatory sequence, a CD28 co-stimulatory sequence, and CD3zeta ITAM 1, 2 and 3 intracellular sequences; and wherein
b) the second polypeptide chain comprising a plurality of regions ordered from the amino terminus to the carboxyl terminus: (i) a BCMA antibody light chain variable region comprising the amino acid sequence of SEQ ID NO: 8, 9, 10, 13, 15, 17, 19, 21, 23, 25, 27 or 30; and (ii) an antibody light chain constant region,
wherein the antibody heavy chain constant region and the antibody light chain constant region form a dimerization domain, and
wherein the antibody heavy chain variable region and the antibody light chain variable region form an antigen binding domain that binds a BCMA protein, optionally wherein the dimeric antigen receptor (DAR) construct is a DAR V2c construct.

44. The dimeric antigen receptor (DAR) construct of claim 27, wherein
a) the first polypeptide chain comprises a plurality of regions ordered from the amino terminus to the carboxyl terminus: (i) a BCMA antibody heavy chain variable region comprising the amino acid sequence selected from a group consisting of SEQ ID NO: 6, 12, 14, 16, 18, 20, 22, 24, 26, or 28; (ii) an antibody heavy chain constant region; (iii) a hinge region comprising a CD28 hinge region; (iv) a CD28 transmembrane region; and (v) an intracellular region comprising a 4-1BB co-stimulatory sequence and a CD3zeta ITAM 3 intracellular sequence, wherein the intracellular region optionally includes an intracellular CD28 co-stimulatory sequence; and wherein
b) the second polypeptide chain comprising a plurality of regions ordered from the amino terminus to the carboxyl terminus: (i) a BCMA antibody light chain variable region comprising the amino acid sequence of SEQ ID NO: 8, 9, 10, 13, 15, 17, 19, 21, 23, 25, 27 or 30; and (ii) an antibody light chain constant region,
wherein the antibody heavy chain constant region and the antibody light chain constant region form a dimerization domain, and
wherein the antibody heavy chain variable region and the antibody light chain variable region form an antigen binding domain that binds a BCMA protein, optionally wherein the dimeric antigen receptor (DAR) construct is a DAR V3 construct.

45. The dimeric antigen receptor (DAR) construct of claim 27, wherein
a) the first polypeptide chain comprises a plurality of regions ordered from the amino terminus to the carboxyl terminus: (i) a BCMA antibody heavy chain variable region comprising the amino acid sequence of SEQ ID NO:6, 12, 14, 16, 18, 20, 22, 24, 26, or 28; (ii) an antibody heavy chain constant region; (iii) a CD28 transmembrane region; and (iv) an intracellular region comprising a 4-1BB co-stimulatory sequence and a CD3zeta ITAM 3 intracellular sequence; and wherein
b) the second polypeptide chain comprising a plurality of regions ordered from the amino terminus to the carboxyl terminus: (i) a BCMA antibody light chain variable region comprising the amino acid sequence of SEQ ID NO:8, 9, 10, 13, 15, 17, 19, 21, 23, 25, 27 or 30; and (ii) an antibody light chain constant region,
wherein the antibody heavy chain constant region and the antibody light chain constant region form a dimerization domain, and
wherein the antibody heavy chain variable region and the antibody light chain variable region form an antigen binding domain that binds a BCMA protein, optionally wherein the dimeric antigen receptor (DAR) construct is a DAR V4 construct.

46. The dimeric antigen receptor (DAR) construct of claim 27, wherein
a) the first polypeptide chain comprises a plurality of regions ordered from the amino terminus to the carboxyl terminus: (i) a BCMA antibody heavy chain variable region comprising the amino acid sequence selected from a group consisting of SEQ ID NO:6, 12, 14, 16, 18, 20, 22, 24, 26, or 28; (ii) an antibody heavy chain constant region; (iii) a hinge region comprising a CD28 hinge region; (iv) a CD28 transmembrane region; and (v) an intracellular region comprising a CD28 co-stimulatory sequence and a CD3zeta ITAM 3 intracellular sequence, wherein the intracellular region optionally includes an intracellular CD28 and 4-1BB co-stimulatory sequence; and wherein
b) the second polypeptide chain comprising a plurality of regions ordered from the amino terminus to the carboxyl terminus: (i) a BCMA antibody light chain variable region comprising the amino acid sequence of SEQ ID NO:8, 9, 10, 13, 15, 17, 19, 21, 23, 25, 27 or 30; and (ii) an antibody light chain constant region,
wherein the antibody heavy chain constant region and the antibody light chain constant region form a dimerization domain, and
wherein the antibody heavy chain variable region and the antibody light chain variable region form an antigen binding domain that binds a BCMA protein, optionally wherein the dimeric antigen receptor (DAR) construct is a DAR V3c construct.

47. The dimeric antigen receptor (DAR) construct of claim 27, wherein
c) the first polypeptide chain comprises a plurality of regions ordered from the amino terminus to the carboxyl terminus: (i) a BCMA antibody heavy chain variable region comprising the amino acid sequence of SEQ ID NO:6, 12, 14, 16, 18, 20, 22, 24, 26, or 28; (ii) an antibody heavy chain constant region; (iii) a CD28 transmembrane region; and (iv) an intracellular region comprising a 4-1BB co-stimulatory sequence and a CD3zeta ITAM 3 intracellular sequence; and wherein
d) the second polypeptide chain comprising a plurality of regions ordered from the amino terminus to the carboxyl terminus: (i) a BCMA antibody light chain variable region comprising the amino acid sequence of SEQ ID NO:8, 9, 10, 13, 15, 17, 19, 21, 23, 25, 27 or 30; and (ii) an antibody light chain constant region,
wherein the antibody heavy chain constant region and the antibody light chain constant region form a dimerization domain, and
wherein the antibody heavy chain variable region and the antibody light chain variable region form an antigen binding domain that binds a BCMA protein, optionally wherein the dimeric antigen receptor (DAR) construct is a DAR V4 construct.

48. The DAR construct of claim 41, wherein the intracellular region comprising the 4-1BB co-stimulatory sequence and CD3zeta ITAM 1, 2 and 3 intracellular sequences comprises the amino acid sequence of SEQ ID NO:49.

49. The DAR construct of claim 42, wherein the intracellular region comprising the CD28 co-stimulatory sequence and CD3zeta ITAM 1, 2 and 3 intracellular sequences comprises the amino acid sequence of SEQ ID NO:50.

50. The DAR construct of claim 43, wherein the intracellular region comprising the 4-1BB co-stimulatory sequence, a CD28 co-stimulatory sequence, and CD3zeta ITAM 1, 2 and 3 intracellular sequences comprises the amino acid sequence of SEQ ID NO:51 or 88.

51. The DAR construct of claim 44, wherein the intracellular region comprising the 4-1BB co-stimulatory sequence and a CD3zeta ITAM 3 intracellular sequence comprises the amino acid sequence of SEQ ID NO:52, or the intracellular region comprising a 4-1BB co-stimulatory sequence, a CD28 co-stimulatory sequence and a CD3zeta ITAM 3 intracellular sequence comprises the amino acid sequence of SEQ ID NO:53 or 89.

52. The DAR construct of claim 45, wherein the intracellular region comprising the 4-1BB co-stimulatory sequence and a CD3zeta ITAM 3 intracellular sequence comprises the amino acid sequence of SEQ ID NO:52.

53. The DAR construct of claim 46, wherein the intracellular region comprising the CD28 co-stimulatory sequence and a CD3zeta ITAM 3 intracellular sequence comprises the amino acid sequence of SEQ ID NO:87.

54. The DAR construct of claim 47, wherein the intracellular region comprising the 4-1BB co-stimulatory sequence and a CD3zeta ITAM 3 intracellular sequence comprises the amino acid sequence of SEQ ID NO:52.

55. The DAR construct of claim 40 or 51, wherein the CD8 and CD28 hinge region comprises the amino acid sequence of SEQ ID NO:36.

56. The DAR construct of any one of claim 41-44, 46-49, or 54, wherein the hinge region comprises a CD28 hinge any one of claims region comprising the amino acid sequence of SEQ ID NO:35.

57. The DAR construct of any one of claim 40, 45, or 50, wherein the CD28 transmembrane region comprises the amino acid sequence of SEQ ID NO:37.

58. The DAR construct of any one of claims 40-55, wherein the heavy chain constant region comprises:
a) a human IgG, IgA, IgD, IgE, or IgM CH1 domain;
b) a human IgG1, IgG2, IgG3, or IgG4 CH1 domain;
c) a human IgG1 domain;
d) an amino acid sequence having at least 95% sequence identity to the amino acid sequence of SEQ ID NO:7 or 29; or
e) the amino acid sequence of SEQ ID NO:7 or 29.

59. The DAR construct of any one of claims 40-56, wherein the light chain constant region comprises:
a) a human Ig kappa constant domain;
b) a human Ig lambda constant domain;
c) an amino acid sequence having at least 95% sequence identity to the amino acid sequence of SEQ ID NO:11 or 31; or
d) the amino acid sequence of SEQ ID NO:11 or 31.

60. A nucleic acid encoding the precursor polypeptide of any one of claims 1-18.

61. An expression vector comprising the nucleic acid of claim 60 operably linked to a promoter.

62. A host cell, or a population of host cells, harboring the nucleic acid of claim 60 operably linked to a promoter, optionally wherein the nucleic acid is present in an expression vector.

63. The host cell or the population of host cells of claim 62, wherein the host cell is a T lymphocyte (e.g., regulatory T cell, gamma-delta T cell or cytotoxic T cell), a NK (natural killer) cell, a macrophages, a dendritic cell, a mast cell, an eosinophil, a B lymphocyte, or a monocyte, or the population of host cells comprises T lymphocytes (e.g., regulatory T cells, gamma-delta T cells or cytotoxic T cells), NK (natural killer) cells, macrophages, dendritic cells, mast cells, eosinophils, B lymphocytes or monocytes.

64. The host cell or the population of host cells of claim 62, wherein the host cell is an autologous host cell or the population comprises autologous host cells.

65. The host cell or the population of host cells of claim 62, wherein the host cell is an allogeneic host cell or the population comprises allogeneic host cells.

66. A method for preparing a population of host cells expressing a plurality of a dimeric antigen receptor (DAR), comprising: culturing the population of host cells of any one of claims 62-65 under conditions suitable for expressing a plurality of the precursor polypeptide by the population of host cells, and suitable for processing the plurality of precursor polypeptides into a plurality of dimeric antigen receptors (DARs) by the population of host cells, wherein the processing by the population of host cells comprises cleaving the plurality of precursor polypeptide into a plurality of first and second polypeptide chains, assembling the plurality of first and second polypeptide chains with each other to form a plurality of dimeric antigen receptors (DARs), and anchoring the plurality of dimeric antigen receptors (DARs) in the cellular membrane of the population of host cells.

67. The method of claim 66, wherein the expression vector directs transient introduction of the nucleic acid encoding the precursor polypeptide into the host cell or the population of host cells.

68. The method of claim 66, wherein the expression vector directs stable insertion of the nucleic acid encoding the precursor polypeptide into the host cells' genome.

69. The method of claim 66, wherein the expression vector directs transcription and/or translation of the nucleic acid encoding the precursor polypeptide in the host cell or the population of host cells.

70. The method of claim 66, wherein the expression vector directs expression of the nucleic acid encoding the precursor polypeptide in the host cell or the population of host cells, wherein expression includes transcription and/or translation of the nucleic acid encoding the precursor polypeptide.

71. A population of host cells comprising a plurality of dimeric antigen receptors (DARs) anchored in the cellular membrane of the population of host cells prepared by the method of any one of claims 66-70.

72. A population of host cells expressing a plurality of a dimeric antigen receptor (DAR) construct according to any one of claims 19-59, wherein the DAR construct is anchored in the cellular membrane of the population of the host cells.

73. A pharmaceutical composition comprising the population of host cells of claim 71 or 72 and a pharmaceutically-acceptable excipient.

74. A method for treating a subject having a disease, disorder or condition associated with detrimental expression of a tumor antigen in the subject, comprising: administering to the subject the population of host cells of claim 71 or 72 or the pharmaceutical composition of claim 73.

75. The method of claim 74, wherein the disease is a hematologic cancer selected from the group consisting of non-Hodgkin's lymphoma (NHL), Burkitt's lymphoma (BL), B chronic lymphocytic leukemia (B-CLL), B and T acute lymphocytic leukemia (ALL), T cell lymphoma (TCL), acute myeloid leukemia (AML), hairy cell leukemia (HCL), Hodgkin's Lymphoma (HL), chronic myeloid leukemia (CML) and multiple myeloma (MM).

76. A first nucleic acid encoding the first polypeptide of any one of claims 19-59.

77. A second nucleic acid encoding the second polypeptide of any one of claims 19-59.

78. A first nucleic acid encoding the first polypeptide and a second nucleic acid encoding the second polypeptide of any one of claims 19-59.

79. A first expression vector comprising the first nucleic acid of claim 78 operably linked to a promoter and a second expression vector comprising the second nucleic acid of claim 78 operably linked to a promoter.

80. An expression vector comprising the first and second nucleic acid of claim 78 operably linked to a promoter.

81. A first host cell, or a first population of host cells, harboring the first expression vector of claim 79.

82. A second host cell, or a second population of host cells, harboring the second expression vector of claim 79.

83. A first host cell, or a first population of host cells, harboring the first expression vector of claim 79, and a second host cell, or a second population of host cells, harboring the second expression vector of claim 79.

84. A host cell, or a population of host cells, harboring the first and second expression vectors of claim 79.

85. A host cell, or a population of host cells, harboring the expression vector of claim 80.

86. The host cell or the population of host cells of any one of claims 81-84, wherein the host cell is a T lymphocyte (e.g., regulatory T cell, gamma-delta T cell or cytotoxic T cell), a NK (natural killer) cell, a macrophages, a dendritic cell, a mast cell, an eosinophil, a B lymphocyte, or a monocyte or the population comprises T lymphocytes (e.g., regulatory T cells, gamma-delta T cells or cytotoxic T cells), NK (natural killer) cells, macrophages, dendritic cells, mast cells, eosinophils, B lymphocytes or monocytes.

87. The host cell or the population of host cells of any one of claims 81-84, wherein the host cell is an autologous host cell or the population comprises autologous host cells.

88. The host cell or the population of host cells of any one of claims 81-84, wherein the host cell is an allogeneic host cell or the population comprises allogeneic host cells.

89. A method for preparing a plurality of a dimeric antigen receptor (DAR), comprising: culturing the first and second population of host cells of claim 83 under conditions suitable for expressing a plurality of the first and second polypeptide chains.

90. A method for preparing a plurality of a dimeric antigen receptor (DAR), comprising: culturing the population of host cells of claim 84 under conditions suitable for expressing a plurality of the first and second polypeptide chains by the population of host cells, and suitable for processing the plurality of first and second polypeptides into a plurality of dimeric antigen receptors (DARs) by the population of host cells, wherein the processing by the population of host cells comprises assembling the plurality of first and second polypeptide chains with each other to form a plurality of dimeric antigen receptors (DARs), and anchoring the plurality of dimeric antigen receptors (DARs) in the cellular membrane of the population of host cells.

91. A method for preparing a plurality of a dimeric antigen receptor (DAR), comprising: culturing the population of host cells of claim 85 under conditions suitable for expressing a plurality of the first and second polypeptide chains by the population of host cells, and suitable for processing the plurality of first and second polypeptides into a plurality of dimeric antigen receptors (DARs) by the population of host cells, wherein the processing by the population of host cells comprises assembling the plurality of first and second polypeptide chains with each other to form a plurality of dimeric antigen receptors (DARs), and anchoring the plurality of dimeric antigen receptors (DARs) in the cellular membrane of the population of host cells.

92. The method of claim 89, wherein the first expression vector directs transient introduction of the nucleic acid encoding the first polypeptide chain into the first host cell or the first population of host cells, and wherein the second expression vector directs transient introduction of the nucleic acid encoding the second polypeptide chain into the second host cell or the second population of host cells.

93. The method of claim 89, wherein the first expression vector directs stable insertion of the nucleic acid encoding the first polypeptide chain into the first host cells' genome, and wherein the second expression vector directs stable insertion of the nucleic acid encoding the second polypeptide chain into the second host cells' genome.

94. The method of claim 89, wherein the first expression vector directs transcription and/or translation of the nucleic acid encoding the first polypeptide chain in the first host cell or the first population of host cells, and wherein the second expression vector directs transcription and/or translation of the nucleic acid encoding the second polypeptide chain in the second host cell or the second population of host cells.

95. The method of claim 90, wherein the first expression vector directs transient introduction of the nucleic acid encoding the first polypeptide chain into the host cell or the population of host cells, and wherein the second expression vector directs transient introduction of the nucleic acid encoding the second polypeptide chain into the host cell or the population of host cells.

96. The method of claim 90, wherein the first expression vector directs stable insertion of the nucleic acid encoding the first polypeptide chain into the host cells' genome, and wherein the second expression vector directs stable insertion of the nucleic acid encoding the second polypeptide chain into the host cells' genome.

97. The method of claim 90, wherein the first expression vector directs transcription and/or translation of the nucleic acid encoding the first polypeptide chain in the host cell or the population of host cells, and wherein the second expression vector directs transcription and/or translation of the nucleic acid encoding the second polypeptide chain in the host cell or the population of host cells.

98. The method of claim 91, wherein the expression vector directs transient introduction of the nucleic acid encoding the first and second polypeptide chains into the host cell or the population of host cells.

99. The method of claim 91, wherein the expression vector directs stable insertion of the nucleic acid encoding the first and second polypeptide chains into the host cells' genome.

100. The method of claim 91, wherein the expression vector directs transcription and/or translation of the nucleic acid encoding the first and second polypeptide chains in the host cell or the population of host cells.

101. A population of host cells comprising a plurality of dimeric antigen receptors (DARs) anchored in the cellular membrane of the population of host cells prepared by the method of any one of claims 90-100.

102. A population of host cells expressing a plurality of a dimeric antigen receptor (DAR) construct according to any one of claims 19-59 anchored in the cellular membrane of the population of the host cells.

103. A pharmaceutical composition comprising the population of host cells of claim 101 or 102 and a pharmaceutically-acceptable excipient.

104. A method for treating a subject having a disease, disorder or condition associated with detrimental expression or over-expression of a tumor antigen in the subject, comprising: administering to the subject the population of host cells of claim 101 or 102 or the pharmaceutical composition of claim 103.

105. The method of claim 104, wherein the disease is a hematologic cancer selected from the group consisting of non-Hodgkin's lymphoma (NHL), Burkitt's lymphoma (BL), B chronic lymphocytic leukemia (B-CLL), B and T acute lymphocytic leukemia (ALL), T cell lymphoma (TCL), acute myeloid leukemia (AML), hairy cell leukemia (HCL), Hodgkin's Lymphoma (HL), chronic myeloid leukemia (CML) and multiple myeloma (MM).
